SEQUENCE LISTING (24 sequences)

SEQ ID NO: 1 - DNA, 1698 bp, Rhizobium meliloti
  CDS (304)..(1431)
gcagtgatggcggcctgggctgcaagctccgtccgtgtcagcgcctggccgcccatcagagcgggcagcatgtttgcgcgtgcgggatcgccgatcaacgagccgatcagagctatgtccgggccttccttcatacttcgatgataatcgaagtatcgcggacgggcaagacccggatcggcggcgcctggacgatgactcctgcggcgacgcaaatttttccggcgccttcaggctttggtaaccatcttcggtaaccataagcctatcgtcactccgactaagcgtatttgcgagttgccaatgtcatcagttgtttcgcttgccgaaatctcccgtgccgcccgtccgctgaactggctggacagcatcatcaagggagattgcgtggccgcgctgaacgcgcttcccgatcattcggtcgatgtcgtcttcgccgacccgccctataatcttcagctcggcggcacgttgcaccggcccgatcagtcgctggtcgatgcagtggacgacgattgggaccagtttgcttccttcgaagcctatgacgctttcacccgcgcctggctgcttgcctgccggcgtgtcctgaagcccaccggcacgctctgggtcatcggttcctaccacaatatcttccgggtcggcgcgatcctccaggacctgcacttctgggtcttgaacgatatcatctggcgcaagacccaacccgatgccgaacttcaagggcgccgcttccagaacgcgcatgaaacgctgatctgggcgacggcgaacgccaaggccaagggttataccttcaactacgaagcgatgaaggcggcgaacgacgacgttcagatgcgctccgactggctgttccccatctgctccggttccgagcggctgaagggcgacgacggcaagaaagtacacccgacgcaaaagccggaagcgctgcttgcccgcatcctgatggcctcgaccaagcccggggacgtcgtgcttgatccgttcttcggctccggcaccaccggggcggtcgccaagcgcctcggccggcacttcgtcgggatcgagcgcgagcaggactatatcgatgccgccgccgaacgtatcgcggccgtggagccgctcggcaaggccacgctctcggtcatgaccggcaagaaggcggagccgcgcgtcgccttcaacactctggtggaaagcgggctcatcaagcccggcacggttctgacggatgcgaagcgccgctacagcgcgatcgtccgcgccgacggcacgctggcgtccggcggcgaggctggatccattcaccgcctcggcgcaaaagtgcagggcctcgacgcctgcaacggctggaccttctggcacttcgaggagggaagcgtattgaaaccgatcgacgagctcagatccgtcattcgaaacgacctggcaaaactgaactgatcaaccacttccgcctgggtcttcgataggcgcccccttccggtttttgtgccttcagtcccggatgagcgctttaaacgccggaatccgaagaggattccggggctttgtatcaatgcggggcgtcagcgttagagcatggcagccgctgccaggagggtgccgcgtccgaagctggataccgtagccgcgagatcggctttcagcctttcggcgccggtgaactgaaccgcgtgccagccggccgctgcgcgccttcgacat

SEQ ID NO: 2 - protein, 376 aa, Rhizobium meliloti
MSSVVSLAEISRAARPLNWLDSIIKGDCVAALNALPDHSVDVVFADPPYNLQLGGTLHRPDQSLVDAVDDDWDQFASFEAYDAFTRAWLLACRRVLKPTGTLWVIGSYHNIFRVGAILQDLHFWVLNDIIWRKTQPDAELQGRRFQNAHETLIWATANAKAKGYTFNYEAMKAANDDVQMRSDWLFPICSGSERLKGDDGKKVHPTQKPEALLARILMASTKPGDVVLDPFFGSGTTGAVAKRLGRHFVGIEREQDYIDAAAERIAAVEPLGKATLSVMTGKKAEPRVAFNTLVESGLIKPGTVLTDAKRRYSAIVRADGTLASGGEAGSIHRLGAKVQGLDACNGWTFWHFEEGSVLKPIDELRSVIRNDLAKLN

SEQ ID NO: 3 - DNA, 1731 bp, Brucella abortus
  CDS (394)..(1524)
aaagggtaggaacagcaccctcaaatatcgattatgacgcaactccgcgaaattcattatcaaactacacccgacaggcacttcactctgcgagcggctgccacacacactgcatcatcgtcatttgccgccggatcatagaccaaaagaaataaccaagccttattgattgcgacatatgccgttccagccttgcacatggatcacgtcgtcacgatgacaactcgataattatctctgccttattgggcgcgcaaaggccgcaaagccgggctttccctgtgatattaagaaaagatttaggatttcaagcacttggcgttaagcgcatatttaccctaggcagtaaccataggaacaagttttttgcgttcacaggtaatcgagtatcccatgtccctagtacgtcttgcgcatgagttgcccatcgaggccccgcgtaccgcctggctcgactccatcatcaaaggtgattgcgtttccgcgctggagcgcctgccggatcattccgtagacgtcatctttgccgatccgccctataatctccagcttggcggcgatctgcaccgtccggatcagtccatggtcagcgccgtggacgatcattgggaccagtttgaaagcttccaggcctatgacgccttcacccgcgcctggctgctcgcctgccgccgtgtgctgaagccgaatggcaccatctgggtcatcggttcctatcacaatattttccgcgtcggcacgcagttgcaggatctgggcttctggctcctcaacgacattgtctggcgcaagaccaatcccatgccgaatttccgtggccgccgtttccagaatgcgcatgaaacgctgatctgggcttcgcgtgagcagaagggcaagggatatactttcaattacgagggcatgaaagcggccaatgacgatgtgcagatgcgttcggactggctgttcccgatctgcaccggcagtgaacgcctgaaggacgagaacggcgacaaggtccacccgacccagaagccggaagcacttctcgcgcgcatcatgatggcttcaagcaagccgggcgacgttattctcgacccattcttcggttccggcacgaccggcgcggtcgccaagcggcttggccgccatttcgtcggcatcgagcgtgaacagccctatatcgacgccgcaaccgcccgcatcaatgccgtggagccgcttggcaaggcggaactcacggtgatgaccggcaagcgcgcagagccgcgcgtggccttcacgagcgtaatggaagcgggccttttgcgtccgggaaccgtgctttgtgatgaacgccgccgttttgccgccattgttcgcgccgatgggacgctgacggccaacggcgaagccggttcaatccatcgtattggcgccagggttcaagggttcgatgcctgcaatggctggaccttctggcactttgaggaaaacggcgtactgaagcctatcgatgccctgcgcaagatcatccgcgaacagatggctgcggcaggtgcataagaaactttaatatcggacgatctccagtaaactctgatagcaaggcgctcgaagttttcaaacttcgggcgccttcattctttcagaaagaaagctgtcgcgcccgcaaatcgtcggccagtttggctgcgctggtaaaatgcaccgcctgccagcccgcttgcttcgcaccttccacattgtgcatcgtgtcatcgatgaaga

SEQ ID NO: 4 - protein, 377 aa, Brucella abortus
MSLVRLAHELPIEAPRTAWLDSIIKGDCVSALERLPDHSVDVIFADPPYNLQLGGDLHRPDQSMVSAVDDHWDQFESFQAYDAFTRAWLLACRRVLKPNGTIWVIGSYHNIFRVGTQLQDLGFWLLNDIVWRKTNPMPNFRGRRFQNAHETLIWASREQKGKGYTFNYEGMKAANDDVQMRSDWLFPICTGSERLKDENGDKVHPTQKPEALLARIMMASSKPGDVILDPFFGSGTTGAVAKRLGRHFVGIEREQPYIDAATARINAVEPLGKAELTVMTGKRAEPRVAFTSVMEAGLLRPGTVLCDERRRFAAIVRADGTLTANGEAGSIHRIGARVQGFDACNGWTFWHFEENGVLKPIDALRKIIREQMAAAGA

SEQ ID NO: 5 - DNA, 255 bp, Agrobacterium tumefaciens
  CDS (1)..(255)
attttcgccgatccgccgtataatctccagcttggcggcaacgtgcaccggcccgatcagtcgctggtcgatgccgttgatgacgaatgggaccagttcgcctccttcgacgcctatgacgccttcacccgcgcctggctgctcgcctgccgccgtgtgctgaaaccgaacggcaccatctgggtcatcggctcctatcacaatatcttccgcgtcggcgccatgctccagaacctcgatttctggatcctcaac

SEQ ID NO: 6 - protein, 85 aa, Agrobacterium tumefaciens
IFADPPYNLQLGGNVHRPDQSLVDAVDDEWDQFASFDAYDAFTRAWLLACRRVLKPNGTIWVIGSYHNIFRVGAMLQNLDFWILN

SEQ ID NO: 7 - DNA, 2093 bp, Helicobacter pylori
  CDS (202)..(1278)
aacgggcatgctttgcgatttgcatttgaacggatcggggagttatgcgtttttgttgtatcgtttaaaataggtggggataggtagcttctatcatttgatgcatttgatgagaacaaagctagggactaaacattaagatagccttaaaacgcttgtgttaaaatggccagagtagcagatataaaaggctagttaatcatggattttttaaaagaaaacttaaacactatcatagagggggattgtttagaaaaattgaaagattttcctaataaaagcgttgattttatctttgctgaccccccatattttatgcaaacagagggagaattgaagcgttttgaaggcacaaaatttcaaggcgttgaggatcattgggataaatttggctcttttgaagaatacgataccttttgtttgggttggttaaaagaatgccaaaggattttaaaagataatggcactatttgtgtgatagggacttttcaaaatatttttagaattggttttcatttgcaaaatttagggttttggatactcaatgatattgtttggtacaagagcaatccggtgcctaattttgctggcaagagactatgcaacgcccatgaaacgcttatttggtgcgctaaacacaaaaacaacaaagttacctttaattataaaacaatgaagtacctcaataacaataaacaagaaaaatcggtttggcaaatccctatttgcatgggtaacgaaaggctaaaagacgcgcaaggtaaaaaagtgcattccacgcaaaaaccagaagcgctcttaaaaaaaatcattttaagcgcgactaaacctaaagacattattttagatcccttttttggcacaggcacaacaggggctgtggctaaatccatgaacaggtattttattggcattgaaaaagattctttttatatcaaagaagcggcaaaacgccttaatagcactagggataaaagcgattttatcactaatttagatttagaaactaaacccccaaaaatccctatgagtcttttaatttctaaacaattactcaaaattggagattttttatactcatctaacaaagaaaaaatttgtcaagttttagaaaacggacaagtgagggataatgaaaactatgaaacttctattcataagatgagcgctaaatatttgaataaaactaaccataatggctggaaatttttttatgcgtattaccaaaatcaatttttattgttagatgaattgcgttatatctgccaaagggactcttaatggactatcaaacctttaacgagatttttaatcgttttgtttttggaacatctaaagcaaaattagttgaaaatattgccgaaaatcctgaacgctatttggggatttttagacccactaagcctaagacaaaactattagaaaatttattgacttctcatgagattaagtttggcgatgcgtttgaatgcttaatagaacaatatttaaaagagcataacttttcacctttatctaaaaaaattccttattacaataaggataaagaaaaaagggaatctttagaattagatcagtttgctaaaaaagataacacatattattttatagaacaaaaaatgcgagatgaccatgacagcaccaaaaagagagggcaaatagataactttgaaaggaaattagaggctttagtccatcgttatggcgaaaacattcaaggctatttttattttatagatgagggtttgaataaaaatcaaaattactataaagaagaattgcaaaaattatctgttgattatggcgtgcctttgagtttgtgttatggtaaggggttgtttgaatctcttaatatcccgcaagtttgggatgaggttttaagccatttagtgcgatggcgtgaaaccttagccgatttagccagtttgaattttgatgaaaatcctttagaaagttttagagaaatcaaagatttagcgccaagcgtttataggaagcttttggataatgatgaaattttcaatcttgtgttaattttattcccagaacaaaaagttttaaaaatgttagtagagcattttagacaacaaaat

SEQ ID NO: 8 - protein, 359 aa, Helicobacter pylori
MDFLKENLNTIIEGDCLEKLKDFPNKSVDFIFADPPYFMQTEGELKRFEGTKFQGVEDHWDKFGSFEEYDTFCLGWLKECQRILKDNGTICVIGTFQNIFRIGFHLQNLGFWILNDIVWYKSNPVPNFAGKRLCNAHETLIWCAKHKNNKVTFNYKTMKYLNNNKQEKSVWQIPICMGNERLKDAQGKKVHSTQKPEALLKKIILSATKPKDIILDPFFGTGTTGAVAKSMNRYFIGIEKDSFYIKEAAKRLNSTRDKSDFITNLDLETKPPKIPMSLLISKQLLKIGDFLYSSNKEKICQVLENGQVRDNENYETSIHKMSAKYLNKTNHNGWKFFYAYYQNQFLLLDELRYICQRDS

SEQ ID NO: 9 - DNA, 20 bp, Artificial
  Description of Artificial Sequence Forward primer (IFADDPPY)
atyttygcbgayccbccbta

SEQ ID NO: 10 - DNA, 17 bp, Artificial
  Description of Artificial Sequence Reverse primer 1 (LDPFFG)
ccraaraavggrtcsag

SEQ ID NO: 11 - DNA, 17 bp, Artificial
  Description of Artificial Sequence Reverse primer 2 (IGIERE)
tcvcgytcratvccrat

SEQ ID NO: 12 - DNA, 17 bp, Artificial
  Description of Artificial Sequence oligonucleotide
actcgcgagtcaacaga

SEQ ID NO: 13 - DNA, 23 bp, Artificial
  Description of Artificial Sequence oligonucleotide
ggctatttctgttgactcgcgag

SEQ ID NO: 14 - DNA, 23 bp, Artificial
  Description of Artificial Sequence oligonucleotide
tcctctcgcgagtcaacagaaat

SEQ ID NO: 15 - DNA, 30 bp, Artificial
  Description of Artificial Sequence oligonucleotide
cgcggatatttctgttgactcgcgagagga

SEQ ID NO: 16 - DNA, 23 bp, Artificial
  Description of Artificial Sequence oligonucleotide
tcctctcgcgagtcaacagaaat

SEQ ID NO: 17 - DNA, 30 bp, Artificial; identical to SEQ ID NO: 15
  Description of Artificial Sequence oligonucleotide

SEQ ID NO: 18 - DNA, 45 bp, Artificial
  Description of Artificial Sequence oligonucleotide
atcctctcgcgagtcaacagaaatatccgctcatcaccgcaagtt

SEQ ID NO: 19 - DNA, 51 bp, Artificial
  Description of Artificial Sequence oligonucleotide
aacggataacttgcggtgatgagcggatatttctgttgactcgcgagagga

SEQ ID NO: 20 - DNA, 45 bp, Artificial; identical to SEQ ID NO: 18
  Description of Artificial Sequence oligonucleotide

SEQ ID NO: 21 - DNA, 51 bp, Artificial
  Description of Artificial Sequence oligonucleotide
aacggaaaacttgcggtgatgagcggatatttctgttgactcgcgagagga

SEQ ID NO: 22 - DNA, 60 bp, Artificial
  Description of Artificial Sequence oligonucleotide
atcctctcgcgagtcaacagaaatatccgcgagtcaccgcaagttttccgtttgaccggc

SEQ ID NO: 23 - DNA, 66 bp, Artificial
  Description of Artificial Sequence oligonucleotide
ggagggtgccggtcaaacggaaaacttgcggtgactcgcggatatttctgttgactcgcgagagga

SEQ ID NO: 24 - DNA, 60 bp, Artificial; identical to SEQ ID NO: 22
  Description of Artificial Sequence oligonucleotide

(19) United States
(12) Patent Application Publication
(10) Pub. No.: US 2005/0227933 A1
(43) Pub. Date: Oct. 13, 2005

(54) Treatment of bacterial induced diseases using DNA methyl transferase inhibitors

(76) Inventors: Stephen J. Benkovic, State College, PA (US); Lucy Shapiro, Stanford, CA (US); Rachel Wright, Menlo Park, CA (US); Craig Stephens, Mountain View, CA (US); Lyn Sue Kahng, Chicago, IL (US); Anthony Berdis, State College, PA (US); Irene Lee, State College, PA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/615,703

(22) Filed: Jul. 9, 2003

Related U.S. Application Data
(63) Continuation-in-part of application No. 09/996,420, filed on Nov. 29, 2001, now abandoned.

(51) Int. Cl. A61K 48/00

(52) U.S. Cl. 514/44

(57) ABSTRACT

Methods for treating and/or preventing disease conditions caused or induced or aggravated by microbes, especially bacteria, by inhibiting DNA methyltransferase activity, such as by administering to an animal a DNA methyltransferase inhibitor, are disclosed, along with methods of reducing or ablating virulence in bacteria by inhibiting DNA methyltransferase activity.

### PRIORITY CLAIM

This application claims priority as a continuation-in-part to U.S. application Ser. No. 09/996,420, filed 29 Nov. 2001; U.S. Ser. No. 09/578,991, filed 25 May 2000, which claimed priority to U.S. provisional patent application Ser. No. 60/135,870, filed 25 May 1999; U.S. provisional patent application Ser. No. 60/154,582, filed 17 Sep. 1999; and U.S. provisional patent application Ser. No. 60/174,256, filed 3 Jan. 2000, and U.S. Ser. No. 09/269,137, filed 16 Mar. 1999, which is a national phase application based on PCT/US97/16593, which claimed priority to U.S. provisional patent application Ser. No. 60/020,089, filed 19 Sep. 1996, the disclosures of which are all hereby incorporated by reference in their entirety.

### GOVERNMENT RIGHTS

The research that led to this application was supported in part by an NIH grant, and the government may have certain rights to the invention.

### FIELD OF THE INVENTION

The present invention relates to the field of treatment and/or prevention of microbe-induced diseases, including bacterially-induced diseases, particularly where such disease treatment and/or prevention comprises administering to an animal an antibiotic compound effective to inhibit bacterial DNA adenine methyltransferase enzymes, including where such antibiotic acts to reduce the virulence of such microbes.

### BACKGROUND OF THE INVENTION

Despite declining incidences of microbial infection, especially by bacteria, misuse of conventional antibiotics and natural selection of the infectious bacterial population has resulted in the development of varying degrees of drug resistance by most bacterial infectious agents to most antibiotic agents. In severe cases, such as MRSA (Multidrug-Resistant StaphA), one or only a few antibiotics are currently effective. In addition, the existence of immunodeficiency syndromes results in additional incidence of opportunistic infections requiring intensive antibiotic treatment.

Thus, there is an increasing need in the art for methods of treating and/or preventing microbial, especially bacterial, infection by means that reduce unwanted side effects on the animal so treated and, in general, for methods that reduce microbial, especially bacterial, virulence.

Most bacteria modify their genomic DNA by methylation of specific nucleotide bases. DNA methylation is critical to gene regulation and repair of mutational lesions (see Jost & Soluz, 1993, DNA METHYLATION, MOLECULAR BIOLOGY AND BIOLOGICAL SIGNIFICANCE, Birhauser Verlag: Basel, Switzerland; Palmer & Marinus, 1994, Gene 143: 1-12; Dryden, 1999, “Bacterial DNA Methyltransferases,” in S-ADENOSYLMETHIONINE-DEPENDENT METHYLTRANSFERASES: STRUCTURES AND FUNCTIONS, X. Cheng and R. M. Blumenthal (eds.), World Scientific Publishing, p. 283-340 for review). DNA methylation is catalyzed by a class of enzymes having different sequence specificities. There are those DNA methyltransferases that methylate nucleotides that are not contained in the recognition site of a cognate restriction enzyme, such as DNA cytosine methyltransferases (dcm) that methylate cytosine residues in CCAGG or CCTGG sequences, or DNA adenine methyltransferases (dam) that methylate adenine residues in GATC sequences. Other DNA methylases methylate residues contained in the recognition site of a cognate restriction enzyme, including ApaI, AvaII, BclI, ClaI, DpnII, EcoRI, HaeIII, HhaI, MboI, and MspI; see, Marinus & Morris, 1973, J. Bacteriol. 114: 1143-1150; May & Hatman, 1975, J. Bacteriol. 123: 768-770; Heitman, 1993, Genet. Eng. 15: 57-108). A DNA methyltransferase from the bacterium Caulobacter crescentus, cell cycle regulated ethyltransferase (“CcrM” refers to the protein and “ccrM” denotes the gene), methylates the adenine residue in the recognition sequence GANTC (where N denotes any nucleotide; Zweiger et al., J. Mol. Biol. 235: 472-485, 1994). CcrM is unusual, as it is not part of a restriction modification system, and is the only known prokaryotic DNA methyltransferase shown to be essential for viability (Stephens et al., 1996, Proc. Natl. Acad. Sci. USA 93:1210-1214) outside of a restriction modification system (i.e., a coexpressed methylase and restriction enzyme which recognize the same nucleotide sequence). The CcrM protein, and therefore its DNA methylation activity, is present only at the predivisional stage of the cell cycle (Zweiger et al., 1994, J. Mol. Biol. 235: 472-485; Stephens et al., 1996, Proc. Natl. Acad. Sci. USA 93:1210-1214). This is controlled in two ways; the ccrM gene is transcribed only in the predivisional cell (Stephens et al., 1995, J. Bacteriol. 177:1662-1669) and the CcrM protein is highly unstable and is completely degraded by the time of cell division in a Lon protease dependent process (Wright et al., 1996, Genes and Development 10:1532-1542).

As described herein, similar methyltransferases occur in Brucella abortus, Helicobacter pylon, Agrobacterium tumefaciens and Rhizobium meliloti. In contrast with bacterial cells, DNA methylation in eukaryotic, and particularly mammalian cells, is limited to cytosine methylation at sites comprising the sequence CpG (Razin & Riggs, 1980, Science 210: 604-610; Jost & Bruhat, 1997, Prog. Nucleic Acid Res. Molec. Biol. 57: 217-248).

Thus, the existence of DNA methylation, in particular, the cell-cycle regulated adenine DNA methyltransferase, presents a novel target for antibiotic action based on an essential protein of the target microbe.

### BRIEF SUMMARY OF THE INVENTION

In one aspect, the present invention relates to a method for treating or preventing a microbe-induced disease in an animal comprising inhibiting DNA methyltransferase activity in said microbe.

In another aspect, the present invention relates to a method of treating or preventing a microbe-induced disease comprising administering to an animal afflicted with said disease, or at risk of becoming afflicted with said disease, a therapeutically effective amount of a DNA adenine methyltransferase inhibitor.

In a further aspect, the present invention relates to a method of reducing bacterial virulence, comprising contacting bacteria with an agent that alters the bacteria's native level of DNA methyltransferase activity thereby inhibiting virulence of the bacteria.

In one preferred embodiment thereof, contacting bacteria with an agent that alters the bacteria's native level of DNA methyltransferase activity results in altering the bacteria's native level of methylation of adenine in a polynucleotide of said bacteria. In another preferred embodiment thereof, contacting bacteria with an agent that alters the bacteria's native level of DNA methyltransferase activity results in altering the bacteria's native level of methylation of adenine in a GATC tetranucleotide of the bacteria. In an additional preferred embodiment thereof, contacting bacteria with an agent that alters the bacteria's native level of DNA methyltransferase activity results in altering the bacteria's native level of methylation of adenine in a GANTC pentanucleotide of the bacteria.

In other such preferred embodiments, the bacteria are pathogenic bacteria that cause disease in a mammal. In an additional preferred embodiment of the methods of the invention the agent reduces the DNA methyltransferase activity, including where the agent reduces said activity by binding to a DNA methyltransferase enzyme.

Preferred embodiments of such methods include those wherein the DNA methyltransferase is a DNA adenine methyl transferase, most preferably wherein said inhibiting DNA methyltransferase activity results from inhibiting DNA methyltransferase enzyme activity or from inhibiting expression of DNA methyltransferase. In a preferred embodiment, the animal is a human patient. In another preferred embodiment, the microbe is a bacterium.

In other preferred embodiments of any of the methods of the invention, the bacterium is a gram positive bacterium, preferably a member selected from the group consisting of Staphylococcus species, Streptococcus species, Bacillus species, Corynebacterium species, Clostridium species, Actinomyces species, Enterococcus species, and Streptomyces species. In another preferred embodiment, the bacterium is a gram negative bacterium, preferably a member selected from the group consisting of Acinetobacter species, Neisseria species, Pseudomonas species, Brucella species, Agrobacterium species, Bordetella species, Escherichia species, Shigella species, Yersinia species, Salmonella species, Klebsiella species, Enterobacter species, Hemophilus species, Pasteurella species, Streptobacillus species, spirochetal species, Campylobacter species, Vibrio species, and Helicobacter species.

### DEFINITIONS

As used herein, the following terms have the indicated meaning unless specifically stated otherwise:

The term “biological activity” in the context of DNA methyltransferase refers to the capacity of the enzyme to act as a methyltransferase as defined herein and is synonymous with DNA methyltransferase activity.

The term “methyltransferase” denotes an enzyme that transfers a methyl group from a methyl donor to a specific site on a nucleic acid substrate, wherein the specific site is preferably a specific base in a characteristic sequence present in the nucleic acid substrate.

The term “processive” methyltransferase signifies that, under the assay conditions used, whenever there is more than one potential methylation site on a DNA substrate, after methylating a first site the methyltransferase methylates the second or subsequent sites without dissociating from the DNA substrate.

The term “DNA-dependent” signifies that the methyltransferase tends to lose activity in solution in the absence of a DNA substrate.

The term “nucleic acid” refers to deoxyribonucleotides or ribonucleotides and polymers thereof in either single- or double-stranded form. Unless specifically limited, the term encompasses nucleic acids containing known analogues of natural nucleotides having similar binding properties as the reference nucleic acid and metabolized in a manner similar to naturally-occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; Cassol et al., 1992, ibid.; Rossolini et al., 1994, Mol. Cell. Probes 8:91-98). The term nucleic acid is used interchangeably with gene, cDNA, and mRNA encoded by a gene.

The phrase “exogenous” or “heterologous” as applied to nucleic acids generally denotes a nucleic acid that has been isolated, cloned and ligated to a nucleic acid with which it is not combined in nature, and/or introduced into and/or expressed in a cell or cellular environment other than the cell or cellular environment in which said nucleic acid or protein may typically be found in nature. The term encompasses both nucleic acids originally obtained from a different organism or cell type than the cell type in which it is expressed, and also nucleic acids that are obtained from the same cell line as the cell line in which it is expressed.

“Nucleic acid probes” may be DNA or RNA fragments. DNA fragments can be prepared, for example, by digesting plasmid DNA, or by use of in vitro amplification methods such as polymerase chain reaction (PCR), or synthesized by either the phosphoramidite method described by Beaucage and Carruthers, 1981, Tetrahedron Lett. 22:1859-1862, or by the triester method according to Matteucci et al., 1981, J. Am. Chem. Soc. 103:3185, both incorporated herein by reference. A double stranded fragment may then be obtained, if desired, by annealing the chemically synthesized single strands together under appropriate conditions or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

The phrase “selectively hybridizing to” refers to a nucleic acid probe that hybridizes, duplexes or binds only to a particular target DNA or RNA sequence when the target sequences are present in a preparation of total cellular DNA or RNA. “Complementary” or “target” nucleic acid sequences refer to those nucleic acid sequences that selectively hybridize to a nucleic acid probe. Proper annealing conditions depend, for example, upon a probe's length, base composition, and the number of mismatches and their position on the probe, and must often be determined empirically. For discussions of nucleic acid probe design and annealing conditions, see, for example, Sambrook et al., 1989, Molecular Cloning: A Laboratory Manual (2nd ed.), Vols. 1-3, Cold Spring Harbor Laboratory, New York, or Current Protocols in Molecular Biology, (F. Ausubel et al., ed.), Greene Publishing and Wiley Interscience, New York (1987).

The phrase “a nucleic acid sequence encoding” refers to a nucleic acid containing sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform with codon preference in a specific host cell.

The term “isolated”, when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated DNA methyltransferase gene is separated from open reading frames that naturally flank the gene and encode a protein other than methyltransferase. The term “purified’ denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “recombinant” or “engineered” when used with reference to a nucleic acid or a protein generally denotes that the composition or primary sequence of said nucleic acid or protein has been altered from the naturally occurring sequence using experimental manipulations well known to those skilled in the art. It may also denote that a nucleic acid or protein has been isolated and cloned into a vector or a nucleic acid that has been introduced into or expressed in a cell or cellular environment, particularly in a cell or cellular environment other than the cell or cellular environment in which said nucleic acid or protein may be found in nature.

The term “recombinant” or “engineered” when used with reference to a cell indicates that the cell replicates or expresses a nucleic acid, or produces a peptide or protein encoded by a nucleic acid, whose origin is exogenous to the cell. Recombinant cells can express nucleic acids that are not found within the native (nonrecombinant) form of the cell. Recombinant cells can also express nucleic acids found in the native form of the cell wherein the nucleic acids are re-introduced into the cell by artificial means.

The following terms are used to describe the sequence relationships between two or more nucleic acids or polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, such as the nucleic acid sequence of SEQ ID NO: 1, 3, 5, or 7, or may comprise a complete cDNA or gene sequence.

Optimal alignment of sequences for aligning a comparison window may be conducted by the local homology algorithm of Smith and Waterman (1981, Adv. Appl. Math. 2:482), by the homology alignment algorithm of Needleman and Wunsch (1970, J. Mol. Biol. 48:443), by the search for similarity method of Pearson and Lipman (1988, Proc. Natl. Acad. Sci USA 85:2444), or by computerized implementations of these algorithms (GAP, BESTFIT, FASTA, and TFASTA in the Wisconsin Genetics Software Package Release 7.0, Genetics Computer Group, 575 Science Dr., Madison, Wis.).

The terms “substantial identity” or “substantial sequence identity”, as applied to nucleic acid sequences and as used herein, denote a characteristic of a polynucleotide sequence, wherein the polynucleotide comprises a sequence that has at least 85 percent sequence identity, preferably at least 90 to 95 percent sequence identity, and more preferably at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence.

As applied to polypeptides, the terms “substantial identity” or “substantial sequence identity” mean that two peptide sequences, when optimally aligned, such as by the programs GAP or BESTFIT using default gap weights, share at least 70 percent sequence identity, preferably at least 80 percent sequence identity, more preferably at least 90 percent sequence identity, and most preferably at least 95 percent amino acid identity or more. “Percentage amino acid identity” or “percentage amino acid sequence identity” refers to a comparison of the amino acids of two polypeptides which, when optimally aligned, have approximately the designated percentage of the same amino acids. For example, “95% amino acid identity” refers to a comparison of the amino acids of two polypeptides which when optimally aligned have 95% amino acid identity. Preferably, non-identical residue positions differ by conservative amino acid substitutions. For example, the substitution of amino acids having similar chemical properties such as charge or polarity are not likely to effect the properties of a protein. Examples include glutamine for asparagine or glutamic acid for aspartic acid.

The term “substantially identical” in the context of two reaction mixtures refers to reaction mixtures that are considered by those of skill to be sufficiently similar that scientifically valid comparisons can be made between them so as to compare relative activity due to the presence or absence of an inhibitor molecule.

A cell has been “transformed” by an exogenous nucleic acid when such exogenous nucleic acid has been introduced inside the cell membrane. Exogenous DNA may or may not be integrated (covalently linked) into chromosomal DNA making up the genome of the cell. The exogenous DNA may be maintained on an episomal element, such as a plasmid. A stably transformed or transfected eukaryotic cell is generally one in which the exogenous DNA has become integrated into the chromosome so that it is inherited by daughter cells through chromosome replication, or one which includes stably maintained extrachromosomal plasmids. This stability is demonstrated by the ability of the eukaryotic cell to establish cell lines or clones comprised of a population of daughter cells containing the exogenous DNA.

“Adenine methyltransferase substrate” refers to a nucleic acid that is acted upon by a DNA methyltransferase to undergo a methylation at an adenine residue. The optimum substrate contains at least one GANTC site and is preferably of a length that promotes ease of manipulation and yields easily resolvable methylation and/or restriction products, preferably a 45 base pair or longer, preferably double-stranded oligonucleotide or plasmid.

The phrase “an essential adenine DNA methyltransferase” indicates that, in the absence of this enzyme activity at the appropriate stage in the cell cycle, organisms that normally express adenine DNA methyltransferase at that stage will die. Enzyme activity may be impaired by a mutation in the enzyme, by the use of antisense nucleic acid, by intracellular proteolysis of the enzyme, or by the contacting a cell or organism with an inhibitor of the enzyme.

“Restriction” denotes the action of hydrolyzing a single or double stranded nucleic acid at a specific sequence or site. “Restriction enzyme” is a nuclease that recognizes a specific sequence or site of a nucleic acid, and cleaves the nucleic acid at that site. “Restriction site” is the particular sequence or site recognized and hydrolyzed by a restriction enzyme.

The phrase “specifically binds to an antibody” or “specifically immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in a sample. Specific binding to an antibody under such conditions may require an antibody that is selected for its specificity for a particular protein. For example, antibodies raised to adenine methyltransferase with the amino acid sequence depicted in SEQ ID NO: 2, 4, 6, or 8 can be selected to obtain antibodies specifically immunoreactive with that adenine methyltransferase and not with other proteins. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane, 1988, Antibodies, A Laboratory Manual, Cold Spring Harbor Publications, New York, for a description of immunoassay formats and conditions that can be used to determine specific immunoreactivity.

By “alkyl”, “lower alkyl”, and “C₁-C₆ alkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By “alkoxy”, “lower alkoxy”, and “C₁-C₆ alkoxy” in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

By the term “halogen” in the present invention is meant fluorine, bromine, chlorine, and iodine.

By “cycloalkyl”, e.g., C₃-C₇ cycloalkyl, in the present invention is meant cycloalkyl groups having 3-7 atoms such as, for example cyclopropyl, cyclobutyl, cyclopentyl, cyclohexyl, and cycloheptyl. In the C₃-C₇ cycloalkyl groups, preferably in the C₅-C₇ cycloalkyl groups, one or two of the carbon atoms forming the ring can optionally be replaced with a hetero atom, such as sulfur, oxygen or nitrogen. Examples of such groups are piperidinyl, piperazinyl, morpholinyl, pyrrolidinyl, imidazolid inyl, oxazol idinyl, azaperhydroepinyl, oxazaperhydroepinyl, oxepanyl, oxazaperhydroinyl, and oxadiazaperhydroinyl. C₃ and C₄ cycloalkyl groups having a member replaced by nitrogen or oxygen include aziridinyl, azetidinyl, oxetanyl, and oxiranyl.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, lower alkyl, lower alkoxy, lower alkylthio, trifluoromethyl, lower acyloxy, aryl, heteroaryl, and hydroxy. Preferred aryl groups include phenyl and naphthyl, each of which is optionally substituted as defined herein.

By “heteroaryl” is meant one or more aromatic ring systems of 5-, 6-, or 7-membered rings containing at least one and up to four heteroatoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, napthyridinyl, benzimidazolyl, benzoxazolyl. Preferred heteroaryls are thiazolyl, pyrimidinyl, preferably pyrimidin-2-yl, and pyridyl. Other preferred heteroaryl groups include 1-imidazolyl, 2-thienyl, 1-, or 2-quinolinyl, 1-, or 2-isoquinolinyl, 1-, or 2-tetrahydro isoquinolinyl, 2- or 3-furanyl and 2-tetrahydrofuranyl.

### BRIEF DESCRIPTION OF THE DRAWINGS

FIGS. 1 and 2 depict schematic diagrams of the “active site” of bacterial adenine DNA methyltransferases.

### DETAILED DESCRIPTION OF THE INVENTION

The present invention relates to a method for treating a microbial-caused disease in a patient afflicted therewith and/or preventing such infection in a patient at risk of becoming so-infected, wherein the methods comprise inhibiting a DNA methyltransferase activity in said microbe, preferably a DNA adenine methyltransferase activity, and most preferably wherein the microbe is a bacterium. In specific embodiments, such inhibiting includes either inhibiting DNA methyltransferase enzyme activity or inhibiting expression, such as transcription or translation, of a DNA methyltransferase.

In a preferred embodiment of such methods, the treatment and/or prevention comprises administering to an animal, such as a human patient, a therapeutically effective amount of any of the compounds disclosed herein as having antimicrobial activity. In a general aspect, the microbes may be bacteria, fungi, viruses and other microbes.

In a further aspect, the present invention relates to methods for reducing bacterial virulence, comprising contacting bacteria with an agent that alters the bacteria's native level of DNA methyltransferase activity thereby inhibiting virulence of the bacteria.

In one preferred embodiment thereof, contacting bacteria with an agent that alters the bacteria's native level of DNA methyltransferase activity results in altering the bacteria's native level of methylation of adenine in a polynucleotide of said bacteria. In another preferred embodiment thereof, contacting bacteria with an agent that alters the bacteria's native level of DNA methyltransferase activity results in altering the bacteria's native level of methylation of adenine in a GATC tetranucleotide of the bacteria. In an additional preferred embodiment thereof, contacting bacteria with an agent that alters the bacteria's native level of DNA methyltransferase activity results in altering the bacteria's native level of methylation of adenine in a GANTC pentanucleotide of the bacteria.

In other such preferred embodiments, the bacteria are pathogenic bacteria that cause disease in a mammal. In an additional preferred embodiment of the methods of the invention the agent reduces the DNA methyltransferase activity, including where the agent reduces said activity by binding to a DNA methyltransferase enzyme.

In a preferred embodiment, the microbe is a bacterium, preferably a gram positive bacterium, wherein said gram positive bacterium is a member selected from the group consisting of Staphylococcus species, Streptococcus species, Bacillus species, Corynebacterium species, Clostridium species, Actinomyces species, Enterococcus species, and Streptomyces species.

In another preferred embodiment of such method, the bacterium is a gram negative bacterium, preferably one selected from the group consisting of Acinetobacter species, Neisseria species, Pseudomonas species, Brucella species, Agrobacterium species, Bordetella species, Escherichia species, Shigella species, Yersinia species, Salmonella species, Klebsiella species, Enterobacter species, Hemophilus species, Pasteurella species, Streptobacillus species, spirochetal species, Campylobacter species, Vibrio species, and Helicobacter species.

In a highly preferred embodiment of the present invention, the bacterium is a member of a species including Staphylococcus aureus; Staphylococcus saprophyticus; Streptococcus pyogenes; Streptococcus agalactiae; Streptococcus pneumoniae; Enterococcus faecalis; Enterococcus faecium; Bacillus anthracis; Acinetobacter baumanii; Corynebacterium diphtheria; Clostridium perfringens; Clostridium botulinum; Clostridium tetani; Neisseria gonorrhoeae; Neisseria meningitidis; Pseudomonas aeruginosa; Legionella pneumophila; Escherichia coli; Yersinia pestis; Haemophilus influenzae; Helicobacter pylon; Campylobacter fetus; Vibrio cholerae; Vibrio parahemolyticus; Trepomena pallidum; Actinomyces israelii; Rickettsia prowazekii; Rickettsia rickettsii; Chlamydia trachomatis; Chlamydia psittaci; Brucella abortus; Agrobacterium tumefaciens; and Francisella tularensis.

In support of such methods there is described and provided herein antibiotics, and specifically antibacterial compounds, that are inhibitors of bacterial adenine DNA methyltransferases. These compounds exhibit antibacterial, growth-inhibitory properties against any bacterial species that produces an adenine DNA methyltransferase. These include adenine DNA methyltransferases that are components of bacterial restriction/modification systems as understood in the art, as well as cell-cycle regulated adenine DNA methyltransferases (CcrM), such as those disclosed in International Application Publication No. WO 98/12206 and U.S. Pat. No. 6,413,751, incorporated by reference. Thus, inhibitors of adenine DNA methyltransferases are particularly described herein.

The adenine DNA methyltransferase inhibitors described herein represent a novel class of broad-spectrum antibiotics. Most bacterial species possess a DNA methyltransferase that is part of a modification apparatus, typically associated with a restriction enzyme, that preserves the integrity of cellular DNA while providing a defense against foreign (most typically viral) DNA. In addition, certain bacteria produce an adenine DNA methyltransferase that is essential for bacterial cell growth.

Medically-important bacterial species that provide appropriate targets for the antibacterial activity of the inhibitors of the invention include gram-positive bacteria, including cocci such as Staphylococcus species and Streptococcus species; bacilli, including Bacillus species, Corynebacterium species and Clostridium species; filamentous bacteria, including Actinomyces species and Streptomyces species; gram-negative bacteria, including cocci such as Neisseria species; bacilli, such as Pseudomonas species, Brucella species, Agrobacterium species, Bordetella species, Escherichia species, Shigella species, Yersinia species, Salmonella species, Klebsiella species, Enterobacter species, Hemophilus species, Pasteurella species, and Streptobacillus species; spirochetal species, Campylobacter species, Vibrio species; and intracellular bacteria including Rickettsiae species and Chlamydia species.

It is an important property of the adenine DNA methyltransferase inhibitors of the invention that the level of activity of these substances with cytosine-specific DNA methyltransferases is low. This is because cytosine-specific DNA methyltransferases occur in mammalian, most particularly human, cells, and it is an advantageous property of the adenine DNA methyltransferases of the invention to have little or no inhibitory activity against mammalian methyltransferases. This property confers upon the molecules provided herein the beneficial property of being bacterial cell specific, and having little antibiotic activity against mammalian, most preferably human, cells. Preferably, the IC₅₀ of these compounds for cytosine-specific DNA methyltransferases is greater than 500 μM.

The inhibitory compounds provided herein are represented by Formula I: 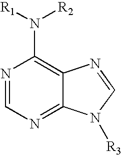
where R¹, R² and R³ are the same or different and are independently hydrogen, lower alkyl, aryl or substituted aryl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from oxygen and nitrogen, and where any member of the alkyl, aryl or cycloalkyl group is optionally substituted with halogen, lower alkyl or lower alkoxy, aryl or substituted aryl, and where R³ can be ribose, deoxyribose or phosphorylated derivatives thereof, including phosphorothioates, phosphoramidites and similar derivatives known in the art, provided that R¹, R² and R³ are not all hydrogen, and where R³ is ribose, deoxyribose or phosphorylated derivatives thereof, R¹ and R² are not both hydrogen. In preferred embodiments, R¹ is H, R² is (2-diphenylborinic ester) ethyl or diphenylpropyl, and R³ is H, 2-(4-morpholinyl)ethyl, 3-(N-phthaloyl)-aminopropyl, 2-(2-(2-hydroxyethoxy)ethoxy)ethyl, or ethyl-2-(acrylate)-methyl. In additional preferred embodiments, R¹ is H, R² is (S-homocysteinyl)methyl and R³ is ribose, 5′phosphorylribose, deoxyribose or 5′ phosphoryl deoxyribose. In other preferred embodiments, R³ is H and R¹ and R² are together 2-(diphenylmethyl)cyclopentyl or 2-(diphenylhydroxymethyl) cyclopentyl. In further preferred embodiments, R¹ is H, R² is alanylbutyl ester, 2-carboximido-2-aminoethyl, 2-aminoethyl or mono- or bisubstituted 2-amino ethyl, and R³ is 2-(4-morpholinyl)-ethyl.

The invention also provides compounds of Formula II: 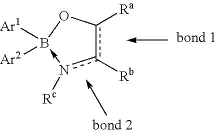
wherein bonds 1 and 2 can be double or single, Ar¹ and Ar² can be the same or different and are each independently aryl or heteroaryl, or aryl or heteroaryl substituted at one or a plurality of positions with halogen, nitro, nitroso, lower alkyl, aryl or substituted aryl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from sulfur, oxygen and nitrogen, and where any member of the alkyl, aryl or cycloalkyl group is optionally substituted with halogen, lower alkyl or lower alkoxy, aryl or substituted aryl, halogen, nitro, nitroso, aldehyde, carboxylic acid, amide, ester, or sulfate, and R^{a}, R^{b} and R^{c} are the same or different and are independently hydrogen, halogen, nitro, nitroso, lower alkyl, lower alkoxy, lower alkoxyalkyl, or cycloalkyl or cycloalkyl alkoxy, or aryl or heteroaryl, or aryl or heteroaryl substituted at one or a plurality of positions with lower alkyl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from sulfur, oxygen and nitrogen, and where any member of the alkyl, aryl or cycloalkyl group is optionally substituted with halogen, lower alkyl or lower alkoxy, aryl or substituted aryl, halogen, nitro, nitroso, aldehyde, carboxylic acid, amide, ester, or sulfate, or wherein R^{a}, R^{b} and R^{c} may be connected by aromatic, aliphatic, heteroaromatic, heteroaliphatic ring structures or substituted embodiments thereof.

It is an important property of the adenine DNA methyltransferase inhibitors of the invention that the level of activity of these substances with cytosine-specific DNA methyltransferases is low. This is because cytosine-specific DNA methyltransferases occur in mammalian, most particularly human, cells, and it is an advantageous property of the adenine DNA methyltransferases of the invention to have little or no inhibitory activity against mammalian methyltransferases. This property confers upon the molecules provided by the invention the beneficial property of being bacterial cell specific, and having little antibiotic activity against mammalian, most preferably human, cells. Preferably, the IC₅₀ of these compounds for cytosine-specific DNA methyltransferases is greater than 500 μM.

The invention also provides compounds of Formula III: 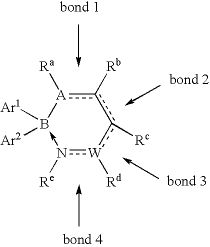
including all pharmaceutically acceptable salts of such compounds,
wherein
   A is N, O or S;
   W is Cₚ, where p is 0 or 1;
   R^{a}, R^{b}, R^{c}, R^{d}, and R^{e} are the same or different and are independently hydrogen, halogen, nitro, nitroso, lower alkyl, aryl or substituted aryl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from sulfur, oxygen and nitrogen, and where any member of the alkyl, aryl or cycloalkyl group is optionally substituted with halogen, lower alkyl or lower alkoxy, aryl or substituted aryl, halogen, nitro, nitroso, aldehyde, carboxylic acid, amide, ester, or sulfate, or wherein R^{a}, R^{b}, R^{c}, R^{d}, and R^{e} may be connected by aromatic, aliphatic, heteroaromatic, heteroaliphatic ring structures or substituted embodiments thereof, where R^{a} is absent when A is O or S and R^{d} is absent when p=0; and
wherein Ar¹ and Ar² can be the same or different and are each independently aryl or aryl substituted at one or a plurality of positions with halogen, nitro, nitroso, lower alkyl, aryl or substituted aryl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from sulfur, oxygen and nitrogen, and where any member of the alkyl, aryl or cycloalkyl group is optionally substituted with halogen, lower alkyl or lower alkoxy, aryl or substituted aryl, halogen, nitro, nitroso, aldehyde, carboxylic acid, amide, ester, or sulfate, and
optionally Ar¹ and Ar² may be cojoined to create a tricyclic scaffold (vide infra), where X=C═O, CHOH, (CH₂)ₙ (n=0 to 2), —CH═CH—, NR^{f} (R^{f}=H, C₁-C₄ alkyl, phenyl, thienyl, or pyridyl), O, SOₙ (n=0 to 2), which have a plurality of positions with halogen, nitro, nitroso, lower alkyl, aryl or substituted aryl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from sulfur, oxygen and nitrogen, and 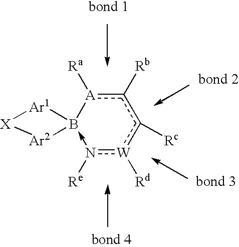
wherein bond 1, bond 2, bond 3 and bond 4 are independently a single bond or a double bond, provided that when A is S or O, bind 1 is a single bond and where A is N, bond 1 is a double bond.

Thus, the invention provides adenine DNA methyltransferase inhibitors that are derivatives of borinic acid, most preferably diphenyl or substituted diphenyl borinic acid, and most preferably diphenyl or substituted diphenyl borinic acid alkylamine esters thereof. In preferred embodiments, the invention provides compounds including di-(4-fluorophenyl)borinic acid 8-hydroxyquinoline ester, di-(4-chlorophenyl)borinic acid 8-hydroxyquinoline ester, di-(3-chlorophenyl)borinic acid 8-hydroxyquinoline ester, di-(4-chloro-2-fluorophenyl)borinic acid 8-hydroxyquinoline ester, di-(3,4-methylenedioxyphenyl)borinic acid 8-hydroxyquinoline ester, di-(4-methoxyphenyl)borinic acid 8-hydroxyquinoline ester, di-(2-thienyl)borinic acid 8-hydroxyquinoline ester, di-(p-fluorophenyl)borinic acid 8-hydroxyquinaldine ester, di-(p-chlorophenyl)borinic acid 8-hydroxyquinaldine ester, di-(4-methoxyphenyl)borinic acid 8-hydroxyquinaldine ester, di-(p-fluorophenyl)borinic acid 5-chloro-8-hydroxyquinaline ester, di-(p-chlorophenyl)borinic acid 5-chloro-8-hydroxyquinaline ester, di-(3,4-methylenedioxyphenyl) borinic acid 5-chloro-8-hydroxyquinoline ester, di-(4-methoxyphenyl)borinic acid 5-chloro-8-hydroxyquinoline ester, di-(3,4-methylenedioxyphenyl)borinic acid 8-hydroxy-5-nitroquinoline ester, diphenylborinic acid 2-aminophenol, diphenylborinic acid pyridine-2-methanol, diphenylborinic acid 2-amino-1-phenylpropanol, diphenylborinic acid (S)-(+)-pyrrolidine-2-methanol, di-(4-fluorophenyl)borinic acid ethanolamine ester, and di-(4-chlorophenyl)borinic acid ethanolamine ester.

Combinatorial chemical libraries of purine derivatives are also provided herein. In one preferred embodiment, 6-chloropurine is converted into adenine derivatives by amination of the C6 position of the purine ring; these libraries are termed “N6 libraries” herein. In other preferred embodiments, unsubstituted adenine or 6-chloropurine is derivatized at the N9 position of the purine ring; these libraries are termed “N9 libraries” herein. In still further embodiments, both the C6 and N9 positions are derivatized, with the C6 position being aminated with an amine or substituted amino group; these libraries are termed “N6/N9 libraries” herein.

In the preparation of the N6 or N9 libraries, the starting purine ring structure is reacted in individual “pots” or reaction mixtures with each of a plurality of amines or substituted amines (for N6 libraries) or halides (for N9 libraries). These libraries thus are provided as collections of separate products of the reaction between the starting materials. For N6/N9 libraries, most preferably the N9 position is first derivatized followed by reaction at the C6 position. In these libraries, reaction is typically performed using a single halide (resulting in uniform substitution at the N9 position) and a plurality of amines (preferably 2 to 5 amines, most preferably 3 different amines), thereby providing a mixture of compounds. In addition, regioisomers (including the N1, N3, and N7 isomers) can be produced according to the methods of the invention. Typically, reaction mixtures are also provided lacking the purine starting material, to monitor for reactions between the halides and the different amines.

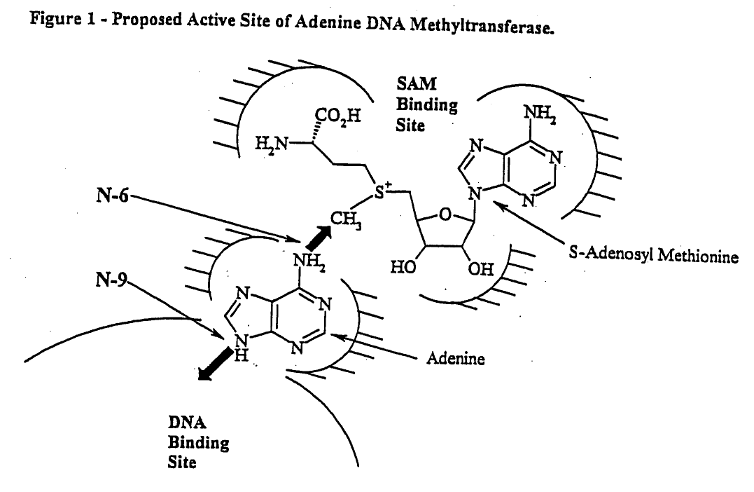

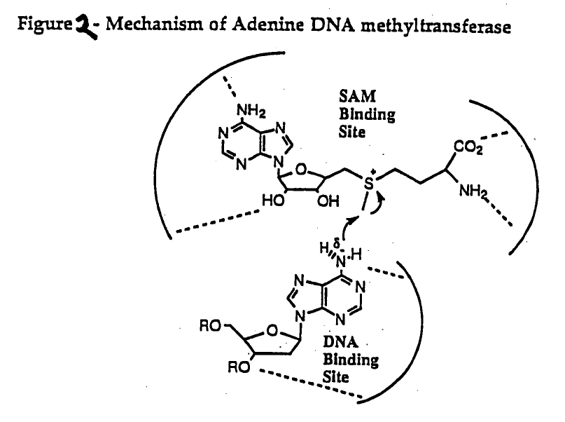

Also provided herein is a so-called “rational design” adenine DNA methyltransferase inhibitors, based on an understanding of the putative active site of an adenine DNA methyltransferase enzyme, shown in FIG. 1. As schematically depicted in the Figure, the enzyme has a binding site for the adenine residue in a DNA strand, and an S-adenosylmethionine binding site, which provides the donor methyl group as shown. So-called “rational design” inhibitors mimic the configuration of the molecules in the binding site of the enzyme, as shown in FIG. 2. These compounds in general comprise an adenosine residue, with or without a 5′ phosphate group, covalently linked through a methylene bridge to a homocysteine moiety.

Also provided are adenine DNA methyltransferase inhibitors synthesized using solid phase chemistry, most preferably using resins comprising a residue (such as an amine or halide) as provided herein for substitution at the C6 or N9 positions of the purine ring. In preferred embodiments, these resins are provided whereby the substituent is covalently linked to the resin using a covalent bond that can be specifically cleaved to liberate the compound from the resin after solid phase synthesis is complete. Preferably, the substituent is presented on the resin with an activated group, such as an amine or halide, accessible to a purine contacted with the resin. After reaction, the purine is linked to the resin through the substituent, and the reaction product can then be worked up and removed from the resin using methods well known in the art. See, for example, Bunin, 1998, THE COMBINATORIAL INDEX, Academic Press.

Also provided are adenine DNA methyltransferase inhibitors that are derivatives of borinic acid, most preferably diphenyl or substituted diphenyl borinic acid, and most preferably diphenyl or substituted diphenyl borinic acid alkylamine esters thereof. In preferred embodiments, the invention provides compounds including di-(p-fluorophenyl)borinic acid 8-hydroxyquiniline ester, di-(p-chlorophenyl)borinic acid 8-hydroxyquiniline ester, diphenylborinic acid 8-hydroxyquiniline ester, di-(p-fluorophenyl)borinic acid ethanolamine ester, and di-(p-chlorophenyl)borinic acid ethanolamine ester.

Advantageously, solid phase chemistry employing resins as described above can be useful for determining whether a substituent exhibits chirality or stereospecificity that has a bearing on antibacterial activity. In these embodiments, compounds are prepared for screening using a racemic mixture of optically-active species, such as an amino acid. Upon finding the resulting compound has adenine DNA methyltransferase inhibitory activity, optically-pure preparations of each of the stereoisomers can be used to prepare the corresponding optically-pure isomers of the adenine DNA methyltransferase inhibitory compound, to determine whether there is any difference in biological activity between the isomers. This approach is advantageous over the alternative, separating the racemic mixture into its stereoisomeric components.

In certain situations, compounds of the invention may contain one or more asymmetric carbon atoms, so that the compounds can exist in different stereoisomeric forms. These compounds can be, for example, racemates or optically active forms. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral HPLC column.

Regardless of how a putative adenine DNA methyltransferase is prepared the compound is analyzed for both adenine and cytosine-specific DNA methyltransferase activity. Susceptible bacteria (known to express an adenine DNA methyltransferase) are grown in the presence and absence of the inhibitory compound, and the extent of growth inhibition in the presence of the compound is determined relative to growth in the absence of the compound. The mechanism of action (i.e., inhibition of adenine DNA methyltransferase) is verified for each growth-inhibitory compound by filter-binding radioassay using hemimethylated DNA, tritiated S-adenosyl methionine (C³H₃) and a purified adenine DNA methyltransferase according to International Application Publication No. WO98/12206.

Compounds described herein can exist as tautomers in solution. When structures and names are given for one tautomeric form the other tautomeric form is also included in the invention.

Representative compounds include, but are not limited to the compounds disclosed herein and their pharmaceutically acceptable acid and base addition salts. In addition, if the compound is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds.

Such compounds may also include the acylated prodrugs. Those skilled in the are will recognize various synthetic methodologies which may be employed to prepare non-toxic pharmaceutically acceptable addition salts and acylated prodrugs of the inventive compounds.

The bacterial growth inhibitory, adenine DNA methyltransferase inhibiting compounds described herein are provided either from combinatorial libraries, solid phase synthesis, “rational” drug design, or conventional synthesis as described herein.

### Construction of Combinatorial Libraries

Combinatorial libraries are prepared according to methods understood by those with skill in the art. For the single substitution libraries (N6 and N9), the individual substituents are used in separate reaction mixtures to produce each of the purine derivatives described herein. In the combination libraries (N6/N9 herein), on the other hand, one position (typically N9) is typically reacted with a particular substituent, and then a mixture of substituents (most preferably 3) used to derivatize the other reaction position (typically C6).

The reactions are performed on a scale adapted to economically producing sufficient product for testing. Preferably, reactions are performed in parallel, for example using a 96-well plate with each well having a sufficiently small volume (100-500 μL) to minimize the amount of reagents required. The use of this type of reaction vessel also facilitates parallel handling and analysis, including automated versions of such processes.

a. N6 Libraries

The following conditions were developed to synthesize analogues of adenine substituted at the N-6 position. In one reaction scheme, 6-chloropurine is reacted at 85° C. overnight with a primary or secondary amine in triethylamine in n-butanol. Alternatively, these reagents are reacted in potassium carbonate in dimethylformamide at 85° C. overnight. This synthesis is described in Reaction Scheme 1. 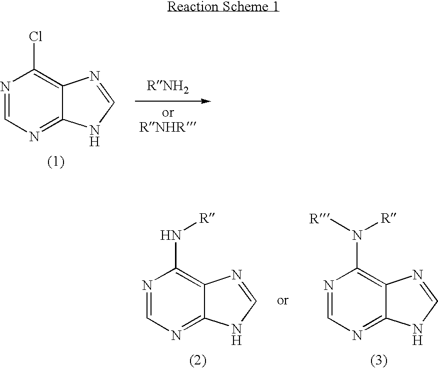
R″ and R′″ are lower alkyl, hetero atom-substituted lower alkyl, aryl, heteroaryl or substituted aryl or heteroaryl, as exemplified by the compounds set forth below. Any primary or secondary amine can be used in this reaction. Preferred embodiments of primary or secondary amines used in these reactions is as follows:
histamine dihydrochloride
norphenylephrine hydrochloride
1,2-diaminopropane
5-amino-1,3,3-trimethylcyclohexanemethyl-amine
3-isopropoxypropylamine
diphenylborinic acid, ethanolamine ester
2-(2-aminoethylamino)-ethanol
tetrahydrofurfurylamine
5-methyltryptamine hydrochloride
3,3-diphenylpropylamine
1-(3-aminopropyl)-2-pyrrolidinone
2-(2-aminoethyl)-1-methylpyrrolidine
2-(aminomethyl)benzimidazole dihydro-chloridehyrate
2,2,2-trifluoroethylamine hydrochloride
L-carnosine
(R)-(−)-1-amino-2-propanol
2-(1-cyclohexenyl)ethylamine
4-(trifluoromethyl)benzylamine
2,5-dichloroamylamine hydrochloride
(+/−)-4-amino-3-hydroxybutyric acid
N,N-dimethylethylenediamine
3,3-dimethylbutylamine
1,4-diamino-2-butanone dihydrochloride
aminomethylbenzoic acid
aminohydroxymethylpropane diol
2-(aminoethyl)pyridine
aminobutanol
adamantamine
aminohexanoic acid
N-benyzylethanolamine
ethyl-6-aminobutyrate hydrochloride
ethylenediamine
2-cyclohex-1-enylethylamine

Some of these amines produce different regioisomers, i.e., for some compounds the amine can be added to the C6 position of 6-chloropurine in different orientations, depending on which reactive moiety comprising the amine covalently bonds to C6. However, the occurrence of these regioisomers is not deleterious, since it merely increases the number of candidate compounds in the library.

b. N9 Libraries

N9 libraries were prepared using the following reaction schemes. It was found that Path A of Reaction Scheme 2 did not yield product with all organic halides (R^{iv}X or R⁴X); alternative Path B was found to form product throughout the range of organic halides tested. In each alternative, the organic halide was reacted with purine (either adenine or 6-chloropurine) at 45° C. overnight in potassium carbonate in dimethylformamide. In Path B, however, the N9-derivatized 6-chloropurine was converted to N9-derivatized adenine by reaction of the product of the first reaction with ammonium hydroxide at 85° C. overnight. Both reactions are performed sequentially in the same reaction mixture. 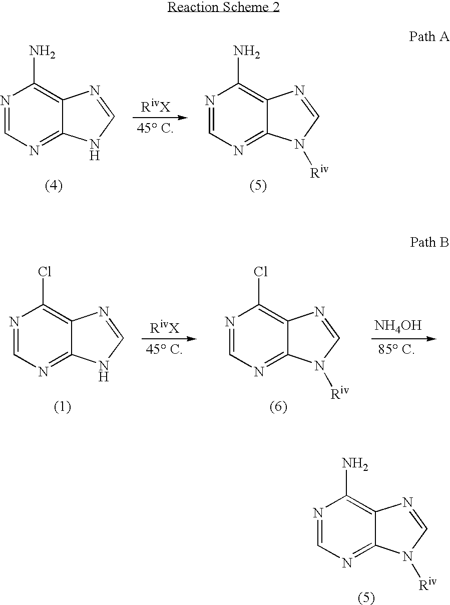
R^{iv} is lower alkyl, hetero atom-substituted lower alkyl, aryl, heteroaryl or substituted aryl or heteroaryl, as exemplified by the compounds set forth below.

The products of Path B were analyzed by HPLC and found to be a mixture of N-9 and N-7 substituted adenine analogues; there may also be N-1 and N-3 substituted analogues in certain reaction mixtures. As discussed above, the advantage of these side products is that their existence simply increases the number of candidate molecules in the library.

Any organic halide can be used in this reaction. Preferred embodiments of organic halides used in these reactions is as follows:
methyl 4-iodobutyrate
1-bromo-3-phenylpropane
cinnamyl bromide
2-chloroethylphosphonic acid
ethyl 2-(2-chloroacetamido)-4-thiazole-acetate
4-(2-chloroethyl)morpholine hydrochloride
(2-bromoethyl)trimethylammonium bromide
4-chlorophenyl 2-bromoethyl ether
N-(3-bromopropyl)pthalimide
2-chloroethyl isocyanate
2-chloro-N,N-dimethylacetoacetamide
3-chloro-2-hydroxypropyl methacrylate
2-bromo-2′-hydroxy-5′-nitroacetanilide
3-(2-bromoethyl)indole
5-chloro-2-pentanone ethylene ketal
2-chloroethyl ethyl sulfide
3-chloro-N-hydroxy-2,2-dimethyl-propionamide
L-1-p-Tosylamino-2-Phenylethyl chloromethyl ketone
2-(2-bromoethyl)-1,3-dioxolane
ethyl 2-(bromomethyl)acrylate
2-(2-(2-chlorethyoxy)ethoxy)ethanol
(3-chloropropyl)trimethoxysilane chloramphenicol
4-(chloromethyl)benzoic acid
bromoethylamine hydrochloride
epibromohydrin
iodopentane
benzyl bromide

c. N6/N9 Combination Libraries

Having developed the reaction schemes shown above for N6 and N9 libraries, combination libraries having substituents at both the C6 amino and N9 positions were prepared using a modification of Path B of Reaction Scheme 2. 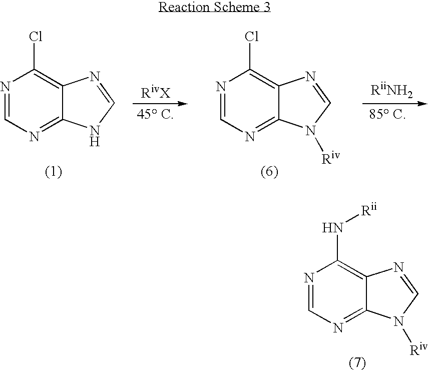
6-Chloropurine was reacted with an organic halide as described above at 45° C. overnight in a solution of potassium carbonate in dimethylformamide. Thereafter, a primary or secondary amine is added to the reaction mixture (although the scheme depicts a primary amine, primary or secondary amines are useful in Reaction Scheme 3) and compound (7) prepared by reaction at 85° C. overnight. In this modification, the primary or secondary amine is substituted in the reaction for ammonium hydroxide show in Path B of Reaction Scheme 2.

Any organic halide can be used in the first step of this reaction. Preferred embodiments of organic halides used in these reactions is as follows:
methyl 4-iodobutyrate
1-bromo-3-phenyl propane
cinnamyl bromide
2-chloroethylphosphonic acid
ethyl 2-(2-chloroacetamido)-4-thiazole-acetate
4-(2-chloroethyl)morpholine hydrochloride
(2-bromoethyl)trimethylammonium bromide
4-chlorophenyl 2-bromoethyl ether
N-(3-bromopropyl)pthalimide
2-chloroethyl isocyanate
2-chloro-N,N-dimethylacetoacetamide
3-chloro-2-hydroxypropyl methacrylate
2-bromo-2′-hydroxy-5′-nitroacetanilide
3-(2-bromoethyl)indole
5-chloro-2-pentanone ethylene ketal
2-chloroethyl ethyl sulfide
3-chloro-N-hydroxy-2,2-dimethyl-propionamide
L-1-p-Tosylamino-2-Phenylethyl chloromethyl ketone
2-(2-bromoethyl)-1,3-dioxolane
ethyl 2-(bromomethyl)acrylate
2-(2-(2-chlorethyoxy)ethoxy)ethanol
(3-chloropropyl)trimethoxysilane
chloramphenicol
4-(chloromethyl)benzoic acid
bromoethylamine hydrochloride
epibromohydrin
iodopentane
benzyl bromide

Any primary or secondary amine can be used in the second step of this reaction. Preferred embodiments of primary or secondary amines used in these reactions is as follows:
histamine dihydrochloride
norphenylephrine hydrochloride
1,2-diaminopropane
5-amino-1,3,3-trimethylcyclohexanemethyl-amine
3-isopropoxypropylamine
diphenylborinic acid, ethanolamine ester
2-(2-aminoethylamino)-ethanol
tetrahydrofurfurylamine
5-methyltryptamine hydrochloride
3,3-diphenylpropylamine
1-(3-aminopropyl)-2-pyrrolidinone
2-(2-aminoethyl)-1-methylpyrrolidine
2-(aminomethyl)benzimidazole dihydro-chloridehyrate
2,2,2-trifluoroethylamine hydrochloride
L-carnosine
(R)-(−)-1-amino-2-propanol
2-(1-cyclohexenyl)ethylamine
4-(trifluoromethyl)benzylamine
2,5-dichloroamylamine hydrochloride
(+/−)-4-amino-3-hydroxybutyric acid
N,N-dimethylethylenediamine
3,3-dimethylbutylamine
1,4-diamino-2-butanone dihydrochloride
aminomethylbenzoic acid
aminohydroxymethylpropane d iol
2-(aminoethyl)pyridine
aminobutanol
adamantamine
aminohexanoic acid
N-benyzylethanolamine
ethyl-6-aminobutyrate hydrochloride
ethylenediamine
2-cyclohex-1-enylethylamine

This chemistry can be repeated with any halide in combination with any amine to give any adenine analogues of this type.

As with Reaction Schemes 1 and 2, different regioisomers are produced with certain N6 amines, and substituted purines are produced at the N1, N3 and N7 positions as well as at the N9 position by reaction with organic halides.

To obtain a complete combinatorial library in a reasonable time the chemistry was performed in a 96 well plate using 80 wells at a time (Columns 1-10, Rows A-H). A single halide was added to the reaction mixture in each row and the first portion of the reaction shown in Reaction Scheme 3 performed overnight as describe above. Sets of three different amines were then added to each well in each of columns 1-7; wells in columns 8, 9 and 10 are prepared containing only one amine, particularly for those species that would have a tendency to react with any additional amines in the reaction mixture. The second portion of the reaction performed overnight. As a result, most of the wells contained a mixture of compounds, each well comprising a set of purine derivatives with a particular substituent at the N9 position and one of three different substituted amino groups at C6. It is recognized that certain of the reaction mixtures will be deficient in one, two or all three of the C6 substituents, depending on the reactivity of each amino group with N9-derivatized 6-chloropurine. In addition, the reaction mixtures contain different regioisomers of these products. Finally, reaction between combinations of the amines reacting directly with the halides without reacting with 6-chloropurine is possible. These product compounds in each reaction mixture were tested as mixtures for antibacterial, and specifically adenine DNA methyltransferase inhibitory activity. Mixtures showing positive results were separated to identify the compound responsible for the result.

### Screening of Combinatorial Libraries

Reaction products from each of the libraries prepared as described above were screened using both in vivo and in vitro screening methods.

In vivo screening methods involved assays for growth inhibition of bacterial cells expressing an adenine DNA methyltransferase essential for cell growth. Advantageously, these screening methods utilize more than one species of bacteria, to identify lead candidates having the broadest spectrum of antibiotic activity. In certain embodiments, the putative inhibitors are first screened against samples of gram positive and gram-negative bacteria; Caulobacter cresentus and Bacillus subtilis are advantageous examples. Additionally advantageous bacterial species for detecting in vivo adenine DNA methyltransferase activity include Helicobacter pylori, Agrobacter tumefaciens, Brucella abortus and Bacillus anthracis.

In these assays, bacterial cultures such as Caulobacter were grown in an appropriate bacterial culture media such as peptone yeast extract (PYE) media (DIFCO) overnight to saturation. Aliquots of this culture were diluted to a concentration having an optical density at 600 nm (OD₆₀₀) of about 0.05. The assay is conveniently performed in 96 well microtitre plates, particularly using libraries prepared in such plates. Using these microtitre plates, an equal amount (100-500 μL) of the diluted bacterial culture was placed in 88 of the 96 wells of the microtitre plate; the remaining 8 wells were used as negative (no bacteria) controls. Eight of the wells were used as positive (no added test compound) controls. For library screening, bacterial aliquots of 146 μL can be used per well with the addition of 4 μL of combinatorial library sample.

A different mixture of library compounds was added to each of the remaining 80 wells per plate, and the cells grown for 24 h at 37° C. Bacterial cell growth was monitored at intervals using a microplate reader to monitor cell growth; cell growth can be monitored by measuring the OD₆₃₀. Wells containing cells growing more slowly than control wells were used to identify corresponding combinatorial library reaction mixtures, which were then synthesized and tested individually to determine the identity of the inhibitory compound.

Using these methods, candidate compounds that inhibited bacterial cell growth at an estimated concentration of <100 μM were identified. Candidate compounds identified from these libraries include 6-N-(diphenylborinic ester)-ethyl-adenine, 6-N-(diphenylborinic ester)-ethyl-9-(2-(4-morpholinyl)-ethyl)-adenine, 6-N-(diphenylborinic ester)-ethyl-9-(3-(N-phthaloyl)-aminopropyl)-adenine, 6-N-(diphenylborinic ester)-ethyl-9-(2-(2-(2-hydroxyethoxy)ethoxy)ethyl)-adenine, and 6-N-(diphenylborinic ester)-ethyl-9-(ethyl-2-acrylate)-methyl-adenine.

In vitro assays were performed directly on reaction mixtures from the combinatorial libraries of the invention, or on candidate compounds identified from the in vivo screening assays described above. These assays are of two types, using purified CcrM methyltransferases from Caulobacter cresentus or Brucella abortus, or using commercially-available preparations of bacterial dam methylases and dcm methylases. In these assays, a synthetic hemimethylated 45/50 DNA substrate (as disclosed in Berdis et al., 1998, Proc. Natl. Acad. Sci. USA 95: 2874-2879) was incubated with the combinatorial library sample containing a putative inhibitor and the methyltransferase at 30° C., and the methyltransferase reaction initiated by the addition of ³H-labeled S-adenosylmethionine (wherein the radioisotope label comprises the transferred methyl group). Inhibition is detected by comparing the amount of radiolabel incorporated in controls where the reaction was performed in the presence and absence of combinatorial library samples; inhibitors cause a reduction in the amount of methylated, ³H-labeled DNA collected on a DE81 filter and radioactivity quantified by liquid scintillation.

Using this assay, four additional compounds from the N6 library were detected that completely inhibited in vitro DNA methylation at an estimated concentration of about 500 μM. These compounds (having the structures shown below) were ribose forms of adenosine having either diphenylborinic acid ethanolamine ester or 3,3-diphenylisopropyl groups covalently linked to the C6 amino group of the adenine ring:

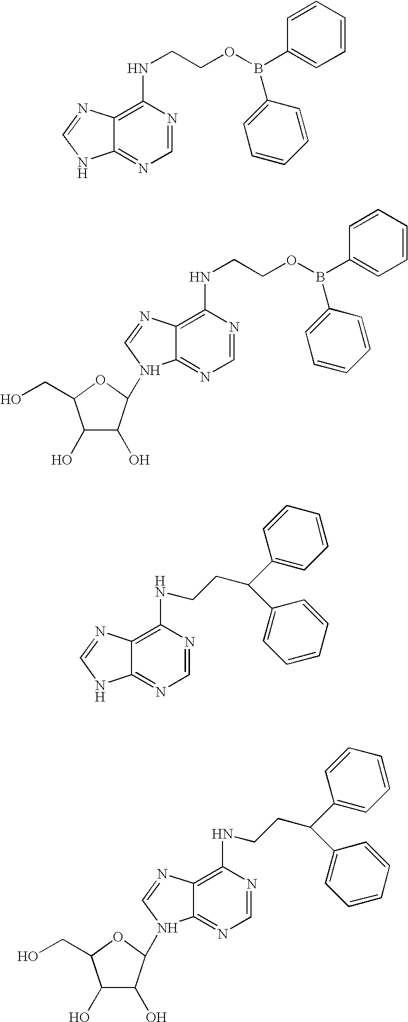

One reaction mixture from the synthesis of the structures shown above from the N-6 adenine library was further analyzed and found to inhibit cell growth at <50 μM. An approximate Kᵢ of 1 μM was measured (assuming the theoretical maximum concentration in the well) for this compound, having the structure:

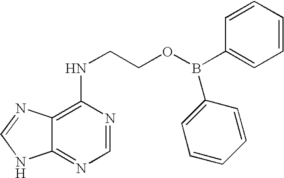

Selected reaction mixtures from the N-9 adenine library were tested for inhibition of adenine DNA methyltransferase activity using this assay. Compounds obtained in the synthesis having N9 substituted with 3-ethylindole or 2-ethyl-1,3-dioxolane (having the structures shown below) were found to be inhibitors of adenine DNA methyltransferase activity at 500 μM:

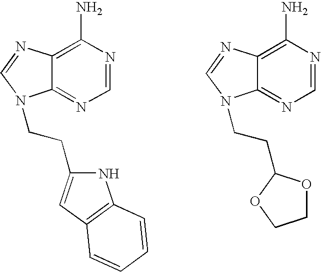

In vitro assays using dcm methyltransferases were performed essentially as described using commercially-available methyltransferase from Hemophilus hemolyticus (New England Biolabs, Beverly, Mass.) and pUC18 DNA as substrate; this assay can also be performed with other commercially-available dcm methyltransferases, for example, from Arthrobacter luteus, Bacillus amyloliquifaciens H, Hemophilus aegyptius, Hemophilus parainfluenza, or Moraxella species. In these assays, adenine specificity is demonstrated by detecting little or no inhibition of the dcm methyltransferase, so that the amount of methylated, ³H-labeled DNA collected on a DE81 filter and radioactivity quantified by liquid scintillation is the same in the presence or absence of the combinatorial library mixtures or putative adenine-specific inhibitor.

Alternatively, assays using dam methylases, for example from Escherichia coli, are performed wherein inhibition is demonstrated by a reduction in the amount of methylated, ³H-labeled DNA was collected on a DE81 filter and radioactivity quantified by liquid scintillation.

Synthesis of “Rational Design” Adenine DNA Methylase Inhibitors

a. Active Site Analogues

“Rational design” of adenine DNA methyltransferases depends on the assumption that the active site of the enzyme contains specific binding sites for the adenine moiety to be modified as well as the S-adenosyl methionine methyl donor, as shown in FIG. 2. Compounds that fit within the active site of the methyltransferase are preferred, and molecules that mimic the putative “transition state” where the methyl transfer activity of the enzyme occurs are particularly preferred. Four such transition state analogues were prepared and assayed in vitro for adenine DNA methyltransferase activity.

The rational design compounds have the structure: 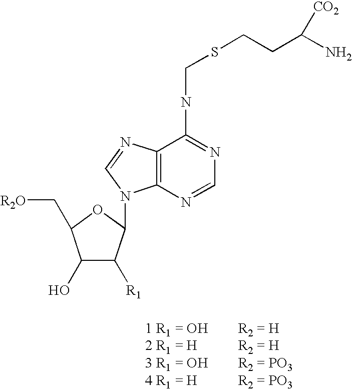
and were synthesized using Reaction Scheme 6, shown below.

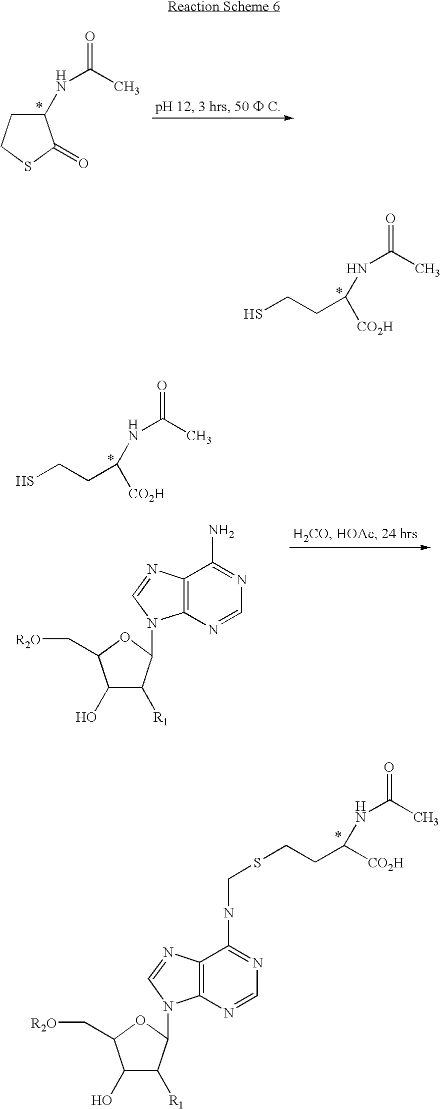

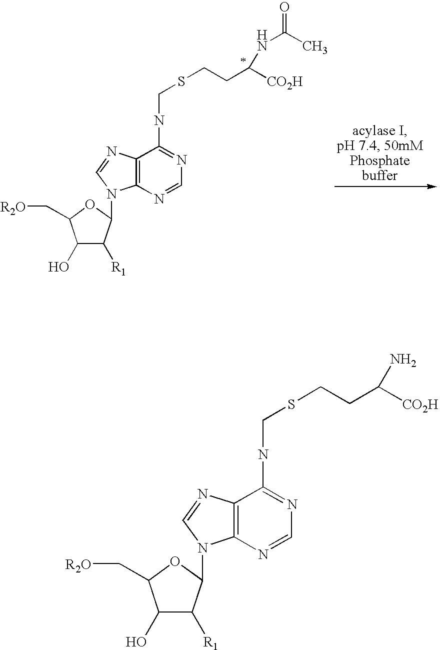

These compounds were assayed as described above for adenine DNA methyltransferase activity. These compounds were found to inhibit CcrM with the Kᵢ's indicated in Table I. The Kᵢ's for compounds 2 and 4 were calculated from a Dixon plot of the data measured at concentration of inhibitors from 0-150 μM for 3 and 0-80 μM for compound 4. Kᵢ's for 1 and 2 are estimated from IC₅₀'s.

TABLE I
K_{I}'s of Compounds 1-4

| Compound | IC₅₀ | Kᵢ |
|---|---|---|
| 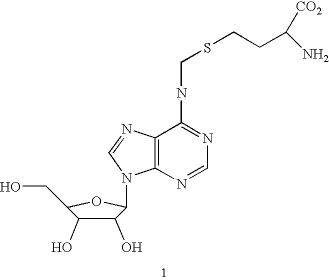 | 2 mM | 160 μM |
| 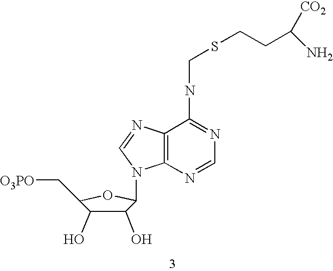 | 1 mM | 80 μM |
| 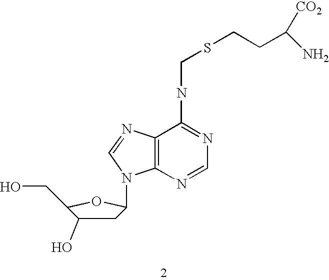 | | 41 μM |
| 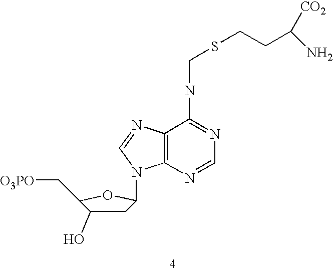 | | 13 μM |

Solid Phase Synthesis of Adenine DNA Methyltransferase Inhibitors

Adenine DNA methyltransferase inhibitors of the invention are also advantageously synthesized using solid phase synthetic methods well known in the art. See Bunin, ibid.

In preferred embodiments, solid phase synthesis complements combinatorial library synthesis as described herein by allowing access to a larger number of library compounds for screening. This synthetic method has the additional advantages of being easier to handle and easier to purify, since they are attached to the resin by chemically-labile groups that can be specifically cleaved.

For example, compound (8) was identified from the N6 library having relatively low (mM range) inhibitory activity:

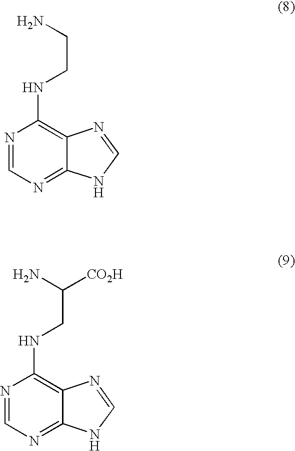

Using these results, second generation libraries were developed using solid phase chemistry to modify inhibitor (8) and its related analogue (9). For example, compound (8) can be derivatized from the terminal amine and from the N-9 position to produce inhibitors that are designed to interact with the S-adenosyl methionine (SAM) and DNA binding sites respectively. Such derivatives can be prepared to target either portion of the methyltransferase active site: modifications of the N9 position are specific to the adenine binding site, while modifications of the C6 amine is specific for the SAM site. Solid phase chemistry was performed using reaction schemes illustrated by Reaction Scheme 4: 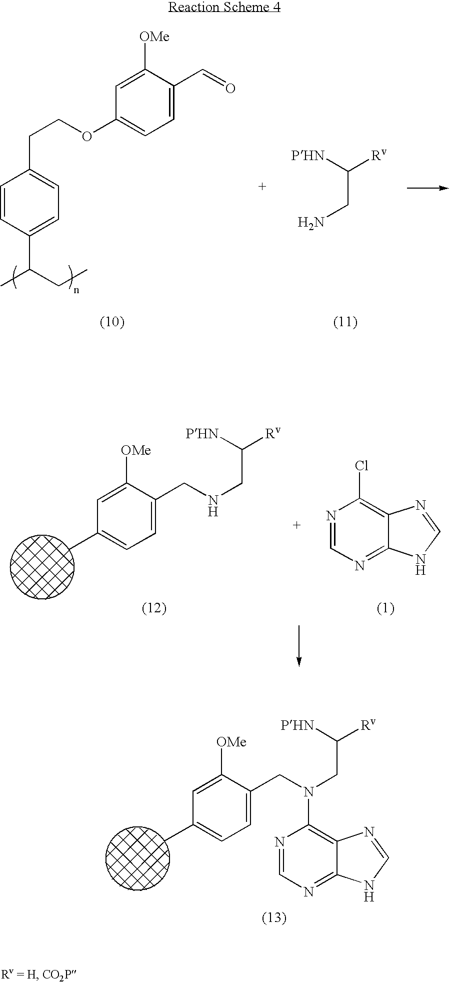
These experiments used a commercially-available methoxyphenyl formyl resin to perform a reductive amination with a protected diamine (11), where R^{v} is hydrogen or CO₂P″ (where P′ and P″ are protecting groups). This reaction yields the secondary amine (12). Addition of 6-chloropurine results in resin-bound adenine adduct (13), permitting the remaining functional groups to be derivatized on the resin. The adducts can be removed from the resin according to art-recognized methods, such as treatment with trifluoroacetic acid, either in concentrated form or as a 5% solution in dichloromethane.

Reaction scheme 4 illustrates an embodiment where the amino substituent contains a chiral center (i.e., it exists as a pair of stereoisomers). However, only one of these stereoisomers may have biological activity. In order to avoid having to separate diastereomeric forms of the compounds of the invention, the following Reaction Scheme 5 can be used:

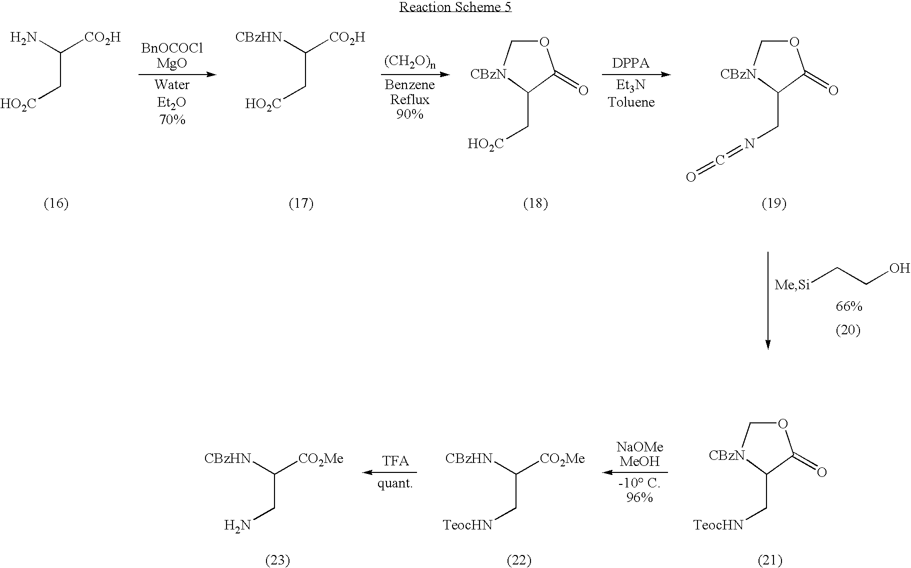

This synthesis can be used to detect adenine DNA methyltransferase inhibitory activity in compounds comprising a racemic mixture of a chiral center (as occurs in aspartic acid (16)); this synthesis can be repeated with commercially pure D- or L-aspartic acid to obtain optically-pure embodiments in the event that one stereoisomer has significantly more activity that the other. As shown in Reaction Scheme 5, D,L-Aspartic acid (16) was treated with benzyl chloroformate to yield the N-carboxybenzyl protected aspartic acid (17). The α-carboxylic acid was then protected as an oxazolidinone (18) using paraformaldehyde. A Curtius rearrangement was performed on the remaining β-carboxylic acid using diphenylphosphoryl azide to give the isocyanate (19). These reactions comprise art-recognized synthetic methods; from this point forward the chemistry is novel. The isocyanate (19) was trapped using trimethylsilyl ethanol (20) to give the Teoc (trimethylsilylethoxycarbonyl) protected amine (21). The oxazolidinone was ring opened using sodium methoxide to give the methyl ester (22). The Teoc protecting group was then removed using trifluoroacetic acid to yield the mono protected diamine (23).

Compounds (23) and (11, where R^{v}=H, P′=CBz) were used to synthesize the resin bound adenine analogues (13), having R^{v}=CO₂Me, P′=CBz, and (13), R^{v}=H, P′=CBz, respectively.

Uses of the Compounds

Provided herein are pharmaceutical compositions of the antibiotics. The pharmaceutical compositions of the present invention can be manufactured in a manner that is itself known, e.g., by means of a conventional mixing, dissolving, granulating, dragee-making, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries that facilitate processing of the active compounds into preparations that can be used pharmaceutically. Proper formulation is dependent upon the route of administration chosen.

Non-toxic pharmaceutical salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—CH₃ where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

For injection, the compounds of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insulator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The antibiotics described herein can be provided as salts with pharmaceutically compatible counterions. Pharmaceutically compatible salts can be formed with many acids, including but not limited to hydrochloric, sulfuric, acetic, lactic, tartaric, malic, succinic, phosphoric, hydrobromic, sulfinic, formic, toluenesulfonic, methanesulfonic, nitric, benzoic, citric, tartaric, maleic, hydroiodic, alkanoic such as acetic, HOOC—(CH₂)ₙ—CH₃ where n is 0-4, and the like. Salts tend to be more soluble in aqueous or other protonic solvents that are the corresponding free base forms. Non-toxic pharmaceutical base addition salts include salts of bases such as sodium, potassium, calcium, ammonium, and the like. Those skilled in the art will recognize a wide variety of non-toxic pharmaceutically acceptable addition salts.

Pharmaceutical compositions of the compounds of the present invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, for example, via injection of the compound directly into a specific tissue, often in a depot or sustained release formulation.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the methods of the invention, the therapeutically effective dose can be estimated initially from cell culture assays, as disclosed herein. For example, a dose can be formulated in animal models to achieve a circulating concentration range that includes the EC50 (effective dose for 50% increase) as determined in cell culture, i.e., the concentration of the test compound which achieves a half-maximal inhibition of bacterial cell growth. Such information can be used to more accurately determine useful doses in humans.

It will be understood, however, that the specific dose level for any particular patient will depend upon a variety of factors including the activity of the specific compound employed, the age, body weight, general health, sex, diet, time of administration, route of administration, and rate of excretion, drug combination, the severity of the particular disease undergoing therapy and the judgment of the prescribing physician.

For administration to non-human animals, the drug or a pharmaceutical composition containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

Preferred compounds of the invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcova et al. (1996, J. Chromat. B 677: 1-27). Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

Dosage amount and interval can be adjusted individually to provide plasma levels of the active moiety that are sufficient to maintain bacterial cell growth inhibitory effects. Usual patient dosages for systemic administration range from 100-2000 mg/day. Stated in terms of patient body surface areas, usual dosages range from 50-910 mg/m²/day. Usual average plasma levels should be maintained within 0.1-1000 μM. In cases of local administration or selective uptake, the effective local concentration of the compound cannot be related to plasma concentration.

The compounds of the invention are modulators of cellular processes in bacteria that infect plants, animals and humans. The pharmaceutical compositions of the adenine DNA methyltransferase inhibitory compounds of the invention are useful as antibiotics for the treatment of diseases of both animals and humans, including but not limited to actinomycosis, anthrax, bacterial dysentery, botulism, brucellosis, cellulitis, cholera, conjunctivitis, cystitis, diphtheria, bacterial endocarditis, epiglottitis, gastroenteritis, glanders, gonorrhea, Legionnaire's disease, leptospirosis, bacterial meningitis, plague, bacterial pneumonia, puerperal sepsis, rheumatic fever, Rocky Mountain spotted fever, scarlet fever, streptococcal pharyngitis, syphilis, tetanus, tularemia, typhoid fever, typhus, and pertussis.

This invention also relates to isolated nucleic acid sequences encoding DNA adenine methyltransferases. DNA methyltransferases are present in gram-negative bacteria such as the free-living bacteria Caulobacter, the agriculturally important nitrogen-fixing bacterium Rhizobium and the highly infectious animal pathogen Brucella. The precise sequences and properties of these methyltransferase genes and enzymes are unknown. Prior to the work summarized herein, it was not clear whether the methyltransferases of other organisms would have homologous sequences and properties.

The procedure for obtaining methyltransferase genes from selected organisms generally involves constructing or obtaining gene libraries from selected organisms, detecting and isolating the desired gene, cloning it, and expressing it in a suitable bacterial strain or transformed cell line.

The nucleic acid compositions of this invention, whether RNA, cDNA, genomic DNA, or a hybrid of the various combinations, may be isolated from natural sources or may be synthesized in vitro. The nucleic acids claimed may be present in transformed or transfected whole cells, in a transformed or transfected cell lysate, or in a partially purified or substantially pure form.

Techniques for nucleic acid manipulation of genes encoding the DNA adenine methyltransferases such as generating libraries, subcloning into expression vectors, labeling probes, DNA hybridization, and the like are described generally in Sambrook, et al., Molecular Cloning—A Laboratory Manual (2nd Ed.), Vol. 1-3, Cold Spring Harbor Laboratory, Cold Spring Harbor, N.Y., 1989, which is incorporated herein by reference. This manual is hereinafter referred to as “Sambrook et al.”

Nucleic acids and proteins are detected and quantified herein by any of a number of means known to those of skill in the art. These include analytical biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like. The detection of nucleic acids proceeds by well-known methods such as Southern analysis, northern analysis, gel electrophoresis, PCR, radiolabeling, scintillation counting, and affinity chromatography.

1. Isolation of Nucleic Acids Encoding DNA Adenine Methyltransferases

There are various methods of isolating the DNA sequences encoding DNA adenine methyltransferases. For example, DNA is isolated from a genomic or cDNA library using labeled oligonucleotide probes (e.g., probes having sequences complementary to the sequences disclosed herein, such as SEQJD NO: 1, 3, 5, 7, 9 and 11). The libraries are generated from DNA and mRNA from cultures of bacteria that are generated from stock cultures. Stock cultures are commercially available from a variety of sources including international depositories such as the American Type Culture Collection.

The probes for surveying the libraries can be used directly in hybridization assays to isolate DNA encoding DNA adenine methyltransferases. Alternatively, probes can be designed for use in amplification techniques such as

PCR, and DNA encoding DNA adenine methyltransferases may be isolated by using methods such as PCR (see below).

Methods for making and screening DNA libraries are well established. See Gubter, U. and Hoffman, B. j. Gene 25:263-269, 1983 and Sambrook, et al. To prepare a genomic library, the DNA is generally extracted from cells and either mechanically sheared or enzymatically digested to yield fragments of about 12-20 kb. The fragments are then separated by gradient centrifugation from undesired sizes and are subcloned in bacteriophage lambda vectors. These vectors and phage are packaged in vitro, as described in Sambrook, et al. The vector is transformed into a recombinant host for propagation, screening and cloning. Recombinant phage are analyzed by plaque hybridization as described in Benton and Davis, Science, 196:180-182 (1977). Colony hybridization is carried out as generally described in M. Grunstein et at. Proc. Natl. Acad. Sci. USA., 72:3961-3965 (1975).

DNA encoding a DNA adenine methyltransferase is identified in either cDNA or genomic libraries by its ability to hybridize with nucleic acid probes, for example on Southern blots, and these DNA regions are isolated by standard methods familiar to those of skill in the art. See Sambrook, et al. The nucleic acid sequences of the invention are typically identical to or show substantial sequence identity (determined as described below) to the nucleic acid sequence of SEQ ID. No. 1, 3, 5, or 7. Nucleic acids encoding DNA adenine methyltransferases will typically hybridize to the nucleic acid sequence of SEQ ID NO: 1, 3, 5, or 7 under stringent conditions. For example, nucleic acids encoding DNA adenine methyltransferases will hybridize to the nucleic acid of sequence ID No. 1 under the hybridization and wash conditions of 50% formamide at 42° C. Other stringent hybridization conditions may also be selected. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may significantly affect the stringency of hybridization, including, among others, base composition and size of the complementary strands, the presence of organic solvents and the extent of base mismatching, the combination of parameters is more important than the absolute measure of any one.

Various methods of amplifying target sequences, such as the polymerase chain reaction, can also be used to prepare DNA encoding DNA adenine methyltransferase. Polymerase chain reaction (PCR) technology is used to amplify such nucleic acid sequences. The isolated sequences encoding DNA adenine methyltransferase may also be used as templates for PCR amplification.

In PCR techniques, oligonucleotide primers complementary to the two 3′ borders of the DNA region to be amplified are synthesized. The polymerase chain reaction is then carried out using the two primers. See PCR Protocols: A Guide to Methods and Applications. (Innis, M, Gelfand, D., Sninsky, J. and White, 1., eds.), Academic Press, San Diego (1990). Primers can be selected to amplify the entire regions encoding a full-length DNA adenine methyltransferase or to amplify smaller DNA segments as desired.

PCR can be used in a variety of protocols to isolate nucleic acids encoding the DNA adenine methyltransferases. In these protocols, appropriate primers and probes for amplifying DNA encoding DNA adenine methyltransferases are generated from analysis of the DNA sequences listed herein. For example, the oligonucleotides of SEQ ID Nos. 9-11 can be used in a PCR protocol as described in example 6 herein to amplify regions of DNA's encoding methyl transferase proteins. Once such regions are PCR-amplified, they can be sequenced and oligonucleotide probes can be prepared from sequence obtained. These probes can then be used to isolate DNA's encoding DNA adenine methyltransferases, similar to the procedure used in examples 6-9 herein. DNA adenine methyltransferases can be isolated from a variety of different cellular sources using this procedure. Other oligonucleotide probes in addition to those of SEQ ID NO: 1, 3, 5, 7 can also be. used in PCR protocols to isolate cDNAs encoding the DNA adenine methyltransferases. Such probes are subsequences of the full-length coding sequences and can be from 20 bases to full length and preferably 30-50 bases in length.

Oligonucleotides for use as probes are chemically synthesized according to the solid phase phosphoramidite triester method first described by Beaucage, S. L. and Carruthers, M. H., 1981, Tetrahedron Lett., 22(20):1859-1862 using an automated synthesizer, as described in Needham-VanDevanter, D. R., et al., 1984, Nucleic Acids Res., 12:6159-6168. Purification of oligonucleotides is by either native acrylamide gel electrophoresis or by anion-exchange HPLC as described in Pearson, J. D. and Regnier, F. E., 1983, 1. Chrom., 255:137-149. The sequence of the synthetic oligonucleotide can be verified using the chemical degradation method of Maxam, A. M. and Gilbert, W. 1980, in Grossman, L. and Moldave, D., eds. Academic Press, New York, Methods in Enzymology, 65:499-560.

Other methods known to those of skill in the art may also be used to isolate DNA encoding the DNA adenine methyltransferase. See Sambrook, et al. for a description of other techniques for the isolation of DNA encoding specific protein molecules.

2. Expression of Methyltransferase

Once DNA encoding DNA adenine methyltransferases is isolated and cloned, one can express the DNA adenine methyltransferases in a variety of recombinantly engineered cells to ascertain that the isolated gene indeed encodes the desired methyltransferase. The expression of natural or synthetic nucleic acids is typically achieved by operably linking a nucleic acid of interest to a promoter (which is either constitutive or inducible), incorporating the construct into an expression vector, and introducing the vector into a suitable host cell. Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both (e.g., shuttle vectors), and selection markers for both prokaryotic and eukaryotic systems. Vectors are suitable for replication and integration in prokaryotes, eukaryotes or preferably both. See, Giliman and Smith (1979), Gene, 8:81-97; Roberts et al. (1987), Nature, 328:731-734; Berger and Kimmel, Guide to Molecular Cloning Techniques, Methods in Enzymology, volume 152, Academic Press, Inc., San Diego, Calif. (Berger); Sambrook et al. (1989), MOLECULAR CLONING—A LABORATORY MANUAL (2nd ed.) Vol. 1-3, Cold Spring Harbor Laboratory, Cold Spring Harbor Press, N.Y., (Sambrook); and F. M. Ausubel et al., CURRENT PROTOCOLS IN MOLECULAR BIOLOGY, eds., Current Protocols, a joint venture between Greene Publishing Associates, Inc. and John Wiley & Sons, Inc., (1994 Supplement) (Ausubel). Product information from manufacturers of biological reagents and experimental equipment also provide information useful in known biological methods. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.), as well as many other commercial sources known to one of skill in the art.

The nucleic acids (e.g., promoters and vectors) used in the present method can be isolated from natural sources, obtained from such sources as ATCC or GenBank libraries, or prepared by synthetic methods. Synthetic nucleic acids can be prepared by a variety of solution or solid phase methods. Detailed descriptions of the procedures for solid phase synthesis of nucleic acids by phosphite-triester, phosphotriester, and H-phosphonate chemistries are widely available. See, for example, Itakura, U.S. Pat. No. 4,401,796; Caruthers, et al., U.S. Pat. Nos. 4,458,066 and 4,500,707; Beaucage, et al., (1981) Tetrahedron Lett., 22:1859-1862; Matteucci, (1981) et al.,). Am. Chem. Soc., 103:3185-3191; Caruthers, et al., (1982) Genetic Engineering, 4:1-17; Jones, chapter 2, Atkinson, et al., chapter 3, and Sproat, et at., chapter 4, in Oligonucleotide Synthesis: A Practical Approach, Gait (ed.), IRL Press, Washington D.C. (1984); Froehler, et al., (1986) Tetrahedron Lett., 27:469-472; Froehier, et al., (1986) Nucleic Acids Res., 14:5399-5407; Sinha, et al. (1983) Tetrahedron Lett., 24:5843-5846; and Sinha, et al., (1984) Nucl. Acids Res., 12:45394557, which are incorporated herein by reference.

a. In Vitro Gene Transfer

It is expected that those of skill in the art are knowledgeable in the numerous expression systems available for expression of DNA encoding DNA adenine methyltransferases. No attempt to describe in detail the various methods known for the expression of proteins in prokaryotes or eukaryotes is made here.

There are several well-established methods of introducing nucleic acids into bacterial and animal cells, any of which may be used in the present invention. These include: calcium phosphate precipitation, fusion of the recipient cells with bacterial protoplasts containing the DNA, treatment of the recipient cells with liposomes containing the DNA, DEAE dextran, receptor-mediated endocytosis, electroporation, micro-injection of the DNA directly into the cells, infection with viral vectors, etc.

For in vitro applications, the delivery of nucleic acids can be to any cell grown in culture, whether of bacterial, plant or animal origin, vertebrate or invertebrate, and of any tissue or type. Contact between the cells and the genetically engineered nucleic acid constructs, when carried out in vitro, takes place in a biologically compatible medium. The concentration of nucleic acid varies widely depending on the particular application, but is generally between about 1 pmol and about 10 mmol. Treatment of the cells with the nucleic acid is generally carried out at physiological temperatures (about 37° C.) for about 1 to about 48 hours, preferably about 2 to 4 hours.

In one group of embodiments, a nucleic acid is added to 60-80% confluent plated cells having a cell density of about 10³ to about 10⁶ cells/mL, more preferably about 2×10′ cells/mL. The concentration of the suspension added to the cells is preferably from about 0.01 to 0.2 pg/mL, more preferably about 0.1 1 ug/mL.

b. Cells to be Transformed

The compositions and methods of the present invention are used to transfer genes into a wide variety of cell types, in vivo and in vitro. Although any prokaryotic or eukaryotic cells may be used, prokaryotic cells such as E. coli are preferred.

c. Detection of Methyltransferase-Encoding Nucleic Acids

The present invention provides methods for detecting DNA or RNA encoding DNA adenine methyltransferases. A variety of methods for specific DNA and RNA measurement using nucleic acid hybridization techniques are known to those of skill in the art. See Sambrook, et al.; NUCLEIC ACID HYBRIDIZATION, A PRACTICAL APPROACH, Ed. Hames, B. D. and Higgins, S. j., IRL Press, 1985; Gall and Pardue (1969), Proc. Natl. Acad. Sci., U.S.A., 63:378-383; and John et al. (1969) Nature, 223:582-587. The selection of a hybridization format is not critical.

For example, one method for evaluating the presence or absence of DNA encoding DNA adenine methyltransferases in a sample involves a Southern transfer. Briefly, the digested genomic DNA is run on agarose slab gels in buffer and transferred to membranes. Hybridization is carried out using the nucleic acid probes discussed above. As described above, nucleic acid probes are designed based on the nucleic acid sequences encoding methyltransferases (See SEQ ID NOs: 1, 3, 5, 7.) The probes can be full length or less than the full length of the nucleic acid sequence encoding the methyltransferase. Shorter probes are empirically tested for specificity. Preferably nucleic acid probes are 20 bases or longer in length. (See Sambrook, et al. for methods of selecting nucleic acid probe sequences for use in nucleic acid hybridization.) Visualization of the hybridized portions allows the qualitative determination of the presence or absence of DNA encoding DNA adenine methyltransferases.

Similarly, a Northern transfer may be used for the detection of mRNA encoding DNA adenine methyltransferases. In brief, the mRNA is isolated from a given cell sample using an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify the presence or absence of DNA adenine methyltransferases.

Sandwich assays are commercially useful hybridization assays for detecting or isolating nucleic acid sequences. Such assays utilize a “capture” nucleic acid covalently immobilized to a solid support and a labelled “signal” nucleic acid in solution. The clinical sample will provide the target nucleic acid. The “capture” nucleic acid and “signal” nucleic acid probe hybridize with the target nucleic acid to form a “sandwich” hybridization complex. To be effective, the signal nucleic acid cannot hybridize with the capture nucleic acid.

Typically, labelled signal nucleic acids are used to detect hybridization. Complementary nucleic acids or signal nucleic acids may be labelled by any one of several methods typically used to detect the presence of hybridized polynucleotides. The most common method of detection is the use of autoradiography with 3H, 1251, 35s, C, or 32P-labelled probes or the like. Other labels include ligands, which bind to labelled antibodies, fluorophores, chemiluminescent agents, enzymes, and antibodies, which can serve as specific binding pair members for a labelled ligand.

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal.

The label may also allow indirect detection of the hybridization complex. For example, where the label is a hapten or antigen, the sample can be detected by using antibodies. In these systems, a signal is generated by attaching fluorescent or enzyme molecules to the antibodies or, in some cases, by attachment to a radioactive label. (Tijssen, P., “Practice and Theory of Enzyme Immunoassays,” Laboratory Techniques in Biochemistry and Molecular Biology, Burdon, R. H., van Knippenberg, P. H., Eds., Elsevier (1985), pp. 9-20.)

The sensitivity of the hybridization assays may be enhanced through use of a nucleic acid amplification system that multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR), the ligase chain reaction (LCR), OJ3-replicase amplification and other RNA polymerase mediated techniques (e.g., NASBA), are found in Berger, Sambrook, and Ausubel, as well as Mullis et al. (1987), U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990), C&EN 3647; The Journal Of NIH Research (1991), 3: 81-94; (Kwoh et al. (1989), Proc. Natl. Acad. Sci. USA, 86:1173; Guatelli et a/. (1990), Proc. Natl. Acad. Sci. USA, 87:t74; Lomell et al. (1989),]. C/in. Chem., 35:1826; Landegren et al. (1988), Science, 241:1077-1080; Van Brunt (1990), Biotechnology, 8:291-294; Wu and Wallace (1989), Gene, 4:560; Barringer et al. (1990), Gene, 89:117, and Sooknanan and Malek (1995), Biotechnology, 13:563-564. Improved methods of cloning in vitro amplified nucleic acids are described in Wallace et al., U.S. Pat. No. 5,426,039. Other methods recently described in the art are the nucleic acid sequence based amplification (NASBATM, Cangene, Mississauga, Ontario) and Q Beta Replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

Oligonucleotides for use as probes, e.g., in in vitro amplification methods, for use as gene probes, or as inhibitor components are typically synthesized chemically according to the solid phase phosphoramidite triester method described by Beaucage and Caruthers (1981), Tetrahedron Letts., 22(20):1859-1862, e.g., using an automated synthesizer, as described in Needham-Van Devanter et al. (1984), Nucleic Acids Res., 12:6159-6168. Purification of oligonucleotides, where necessary, is typically performed by either native acrylamide gel electrophoresis or by anion-exchange HPLC as described in Pearson and Regnier (1983), 1. Chrom., 255:137-149. The sequence of the synthetic oligonucleotides can be verified using the chemical degradation method of Maxam and Gilbert (1980) in Grossman and Moldave (eds.) Academic Press, New York, Methods in Enzymology, 65:499-560.

An alternative means for determining the level of expression of a gene encoding an DNA adenine methyltransferase is in situ hybridization. In situ hybridization assays are well known and are generally described in Angerer, et al., Methods Enzymol., 152:649-660 (1987). In an in situ hybridization assay, cells are fixed to a solid support, typically a glass slide. If DNA is to be probed, the cells are denatured with heat or alkali. The cells are then contacted with a hybridization solution at a moderate temperature to permit annealing of labeled probes specific to DNA adenine methyl transferases. The probes are preferably labeled with radioisotopes or fluorescent reporters.

d. Detection of Methyltransferase Gene Products

Methyltransferase may be detected or quantified by a variety of methods. Preferred methods involve the use of specific antibodies.

Methods of producing polyclonal and monoclonal antibodies are known to those of skill in the art. See, e.g., Coligan (1991), CURRENT PROTOCOLS IN IMMUNOLOGY, Wiley/Greene, NY; and Harlow and Lane (1989), ANTIBODIES: A LABORATORY MANUAL, Cold Spring Harbor Press, NY; Stites et al. (eds.) BASIC AND LINICAL IMMUNOLOGY (4th ed.) Lange Medical Publications, Los Altos, Calif., and references cited therein; Goding (1986), MONOCLONAL ANTIBODIES: PRINCIPLES AND PRACTICE (2d ed.) Academic Press, New York, N.Y.; and Kohler and Milstein (1975), Nature, 256:495-497. Such techniques include antibody preparation by selection of antibodies from libraries of recombinant antibodies in phage or similar vectors. See, Huse et al. (1989), Science, 246:1275-1281; and Ward et al. (1989), Nature, 341:544-546. For example, in order to produce antisera for use in an immunoassay, the polypeptide of SEQ ID NO: 2, 4, 6, or 8, or a fragment thereof, is isolated as described herein. For example, recombinant protein is produced in a transformed cell line. An inbred strain of mice or rabbits is immunized with the protein of SEQ ID No. 2, 4, 6, or 8, or a fragment thereof, using a standard adjuvant, such as Freund's adjuvant, and a standard immunization protocol. Alternatively, a synthetic peptide derived from the sequences disclosed herein and conjugated to a carrier protein can be used as an immunogen. Polyclonal sera are collected and titered against the immunogen protein in an immunoassay, for example, a solid phase immunoassay with the immunogen immobilized on a solid support. Polyclonal antisera with a titer of IO or greater are selected and tested for their cross reactivity against non-adenine methyltransferases or even other adenine methyltransferases, using a competitive binding immunoassay. Specific monoclonal and polyclonal antibodies and antisera will usually bind with a KD of at least about 0.1 mM, more usually at least about 1 pM, preferably at least about 0.1 pM or better, and most preferably, 0.01 pM or better.

A number of immunogens may be used to produce antibodies specifically reactive with DNA adenine methyltransferases. Recombinant protein is the preferred immunogen for the production of monoclonal or polyclonal antibodies. Naturally occurring protein may also be used either in pure or impure form. Synthetic peptides made using the DNA adenine methyltransferase sequences described herein may also used as an immunogen for the production of antibodies to the protein. Recombinant protein can be expressed in eukaryotic or prokaryotic cells as described above, and purified as generally described above. The product is then injected into an animal capable of producing antibodies. Either monoclonal or polyclonal antibodies may be generated, for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to the DNA adenine methyltransferase. When appropriately high titers of antibody to the immunogen are obtained, blood is collected from the animal and antisera are prepared. Further fractionation of the antisera to enrich for antibodies reactive to the protein can be done if desired. (See Harlow and Lane, supra).

Monoclonal antibodies may be obtained by various techniques familiar to those skilled in the art. Briefly, spleen cells from an animal immunized with a desired antigen are immortalized, commonly by fusion with a myeloma cell (See, Kohier and Milstein, Eur. J. Immunol. 6:511-519 (1976), incorporated herein by reference). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al. (1989) Science 246:12751281.

A particular protein can be measured by a variety of immunoassay methods. For a review of immunological and immunoassay procedures in general, see Basic and Clinical Immunology 7th Edition (D. Stites and A. Terr ed.) 1991. Moreover, the immunoassays of the present invention can be performed in any of several configurations, which are reviewed extensively in Enzyme Immunoassay, E. T. Maggio, ed., CRC Press, Boca Raton, Fla. (1980); “Practice and Theory of Enzyme Immunoassays,” P. Tijssen, Laboratory Techniques in Biochemistry and Molecular Biology, Elsevier Science Publishers B. V. Amsterdam (1985); and Harlow and Lane, Antibodies, A Laboratory Manual, supra, each of which is incorporated herein by reference.

Immunoassays to methyltransferases of the present invention may use a polyclonal antiserum which was raised to the protein of SEQ ID NO: 2, 4, 6, or 8, or a fragment thereof. This antiserum is selected to have low crossreactivity against other (non-methyltransferase or methyltransferase) proteins and any such crossreactivity is removed by immunoabsorbtion prior to use in the immunoassay.

In addition, it is possible to produce monospecific antibodies that react to specific DNA methyltransferases from specific species of bacteria as identified herein. Monospecific antibodies are achieved by appropriate cross-absorption with select DNA methyltransferases or by raising antibodies against species-specific regions of the amino acid sequence of the transferases. Such unique peptide fragments are routinely identified by sequence comparisons.

In order to produce antisera for use in an immunoassay, the protein of SEQ ID NO: 2, 4, 6, or 8, or a fragment thereof, is isolated as described herein. For example, recombinant protein is produced in a transformed cell line. An inbred strain of mice such as Balb/c is immunized with the protein of SEQ ID NO: 2 using a standard adjuvant, such as Freund's adjuvant, and a standard mouse immunization protocol. Alternatively, a synthetic peptide derived from the sequences disclosed herein and conjugated to a carrier protein can be used as an immunogen. Polyclonal sera are collected and titered against the immunogen protein in an immunoassay, for example, a solid phase immunoassay with the immunogen immobilized on a solid support. Polyclonal antisera with a titer of 104 or greater are selected and tested for their cross reactivity against non-adenine methyltransferases, using a competitive binding immunoassay such as the one described in Harlow and Lane, supra, at pages 570-573.

Immunoassays in the competitive binding format can be used for the crossreactivity determinations. For example, the protein of SEQ ID NO: 2 can be immobilized to a solid support. Proteins (other methyltransferases, or non-methyltransferases) are added to the assay which compete with the binding of the antisera to the immobilized antigen. The ability of the above proteins to compete with the binding of the antisera to the immobilized protein is compared to the protein of SEQ ID NO: 2. The percent crossreactivity for the above proteins is calculated, using standard calculations. Those antisera with less than 10% crossreactivity with each of the proteins listed above are selected and pooled. The cross-reacting antibodies are optionally removed from the pooled antisera by immunoabsorbtion with the above-listed proteins.

The immunoabsorbed and pooled antisera are then used in a competitive binding immunoassay as described above to compare a second protein to the immunogen protein, in this case, the adenine methyltransferase of SEQ ID NO: 2. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein of SEQ ID NO: 2 that is required, then the second protein is said to specifically bind to an antibody generated to an immunogen consisting of the protein of SEQ ID NO: 2.

The presence of a desired polypeptide (including peptide, transcript, or enzymatic digestion product) in a sample may be detected and quantified using Western blot analysis. The technique generally comprises separating sample products by gel electrophoresis on the basis of molecular weight, transferring the separated proteins to a suitable solid support (such as a nitrocellulose filter, a nylon filter, or derivatized nylon filter), and incubating the sample with labeling antibodies that specifically bind to the analyte protein. The labeling antibodies specifically bind to analyte on the solid support. These antibodies are directly labeled, or alternatively are subsequently detected using labeling agents such as antibodies (e.g., labeled sheep anti-mouse antibodies where the antibody to an analyte is a murine antibody) that specifically bind to the labeling antibody.

3. Purification of DNA Adenine Methyltransferases

The polypeptides described herein may be purified to substantial purity by standard techniques, including selective precipitation with such substances as ammonium sulfate, column chromatography, immunopurification methods, and others. See, for instance, R. Scopes, Protein Purification: Principles and Practice, Springer-Verlag: New York (1982), incorporated herein by reference. For example, the methyltransferase proteins and polypeptides produced by recombinant DNA technology may be purified by a combination of cell lysis (e.g., sonication) and affinity chromatography or immunoprecipitation with a specific antibody to methyltransferase. For fusion products, subsequent digestion of the fusion protein with an appropriate proteolytic enzyme releases the desired polypeptide. The proteins may then be further purified by standard protein chemistry techniques. A specific protocol for purifying the methyltransferases of this invention is provided in Example 6(e).

4. Screening for Inhibitors of Methyltransferase or associated gene Expression

The methyltransferase genes identified herein provide novel targets for screening for agents that attenuate, inhibit, or interfere with the viability of the pathogens bearing with the gene. Inhibition (i.e. blocking) or complete elimination of the expression of the methyltransferase gene or genes described herein results in a mitigation or elimination of the ability of the subject bacteria to infect and/or grow and/or proliferate in an animal or plant host as compared to the same stain of bacteria (or virus) in which there is no inhibition or elimination of the virulence-related gene or gene product.

Having provided herein genes whose expression is required for viability of pathogenic bacteria, it is possible to screen for agents and/or for drugs that, by blocking the activity of the methyltransferase gene, mitigate the virulence of the target pathogen.

Antibiotics and other synthetic drugs targeted to specific proteins generally act by interacting with and inhibiting the activity of the target protein. The methyltransferase enzymatic activity assays provided herein are useful to identify inhibitors of that activity. To do so, the enzymes capacity to methylate a nucleic acid is assayed in the presence and absence of a test substance, such as a synthetic or isolated naturally occurring chemical inhibitor (in particular peptides or other ligands that bind to the active site or to allosteric sites of the methyltransferase enzyme). An inhibitor of the transferase depresses the activity of enzyme at least 50%, preferably at least 90%, and most preferably at least 99%.

The methyltransferase genes or gene product (i.e., mRNA) is preferably detected and/or quantified in a biological sample. As used herein, a biological sample is a sample of biological tissue or fluid that, in a healthy and/or pathological state, contains methyltransferase encoding nucleic acid or the polypeptide. Such samples include, but are not limited to, sputum, amniotic fluid, blood, blood cells (e.g., white cells), tissue or fine needle biopsy samples, urine, peritoneal fluid, and pleural fluid, or cells therefrom. For plants, root tissue or leaf tissue can be used. Biological samples may also include sections of tissues such as frozen sections taken for histological purposes.

The present invention encompasses developing antisense protocols, antibiotics and antagonists that specifically inhibit the methyltransferase activity of the identified enzymes or the expression of the genes of this invention. The detection and testing of such inhibitors is made possible by the ability to make and obtain the claimed enzyme using methods described herein.

Antisense agents are used to reduce or eliminate methyltransferase activity. Antisense agents include fragments or the methyltransferase genes that are operably linked in reverse orientation to an efficient promoter. Also included in antisense agents are ribozymes such as the hairpin or hammerhead types. For antisense agents suitable assays involve detecting the presence, absence, or quantity or amount of transcript of the gene or gene product. Northern blots, quantitative PCR or immunoassays are all suitable for detection of the effectiveness of antisense agents.

In still another embodiment, bacterial reporter strains are used to evaluate candidate anti-transferase agents. In such assays, recombinant bacteria are modified to include a reporter gene attached to a nucleic acid encoding the methyltransferase gene. When the genes are expressed, the reporter gene is also expressed and provides a detectable signal indicating the expression of the gene. Anti-methyltransferase agent screens then involve contacting the reporter strains and/or cells, tissues, or organisms prior to or after infection with the reporter strains and subsequently detecting expression levels of the reporter gene.

In addition to screening for antisense agents, this invention provides for methods that facilitate the identification of non-antisense drug candidates especially under conditions of high throughput. The screening for such non-nucleic acid based inhibitory agents commonly involves contacting the target pathogen (e.g. Brucella abortus), and/or a tissue containing the pathogen, and/or an animal, with one or more candidate anti-methyltransferase agents and detecting the presence absence, quantity of the gene product. Alternatively, candidate anti-methyltransferase agents can be identified simply by their ability to bind to the gene or gene product and inhibit its biological activity.

Methods for detecting the biological activity of the methyltransferases are provided herein and include reaction conditions and suitable substrates for methylation. These assays can be used to screen for anti-methyltransferase agents. Absence of the activity of the gene during and/or after contacting of the bacteria, a cell, a tissue, and/or an organism with an anti-transferase agent of interest will indicate that the particular test compound is a likely candidate for an antibiotic.

In view of the foregoing, preferred assays for detection anti-methyltransferase agents fall into the following categories:
i) Detection of gene or gene-derived nucleic acid presence, absence, or quantity;
ii) Screening for agents that bind to a gene or gene derived nucleic acid;
iii) Detection of a virulence gene derived polypeptide;
iv) Detection of binding of a prospective agent to gene derived polypeptides;
v) Use of bacterial reporter strains; and,
vi) Detection of the biological activity of the transferase gene.

5. High-Throughput Screening of Candidate Agents that Block Methyltransferase Activity.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

a. Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. I. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992)], Amer. Chem. Soc. 114: 6568), nonpeptidal peptidomimetics with a Beta-D-Glucose scaffolding (Hirschmann et al., (1992) 1. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) 1. Org. Chem. 59: 658). See, generally, Gordon et al., (1994) 1. Med. Chem. 37:1385, nucleic acid libraries, peptide nucleic acid libraries (see, e.g., U.S. Pat. No. 5,539,083) antibody libraries (see, e.g., Vaughn et al. (1996) Nature Biotechnology, 14(3): 309-314), and PCT/US96/1 0287), carbohydrate libraries (see, e.g., Liang et al., (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazariones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well-known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

b. High Throughput Assays of Chemical Libraries

Any of the assays for compounds inhibiting the virulence described herein are amenable to high throughput screening. As described above, having identified the nucleic acid associated with virulence, likely drug candidates either inhibit expression of the gene product, or inhibit the activity of the expressed protein. Preferred assays thus detect inhibition of transcription (i.e., inhibition of mRNA production) by the test compound(s), inhibition of protein expression by the test compound(s), or binding to the gene (e.g., gDNA, or cDNA) or gene product (e.g. 1 mRNA or expressed protein) by the test compound(s). Alternatively, the assay can detect inhibition of the characteristic activity of the gene product or inhibition of or binding to a receptor or other transduction molecule that interacts with the gene product.

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high thruput and rapid start up as well as a high degree-of flexibility and customization. The manufacturers of such systems provide detailed protocols the various high throughput. Thus, for example, Zymark Corp. provides technical bulletins describing screening systems for detecting the modulation of gene transcription, ligand binding, and the like.

6. Methyltransferase Activity.

This protocol exemplifies a method for assaying for methyltransferase activity. It is a particularly good method because it allows for the detection of processivity but it need not be so used.

A hemimethylated DNA substrate containing two (2) GANTC methylation sites, for example the N660166-mer described in Example 10(b) below, is used to address the processivity of CcrM. The GANTC sites are resistant to Hinfl digestion but susceptible to Hindu digestion when hemimethylated. However, upon enzymatic methylation, the GANTC sites become fully methylated and resistant to Hindu digestion. The methylation sites in the hemimethylated N660/66-mer substrate are asymmetrically spaced so that DNA fragments of differing sizes are obtained upon Hindu digestion. Thus, one can address the preference for initial methylation by the enzyme during processive DNA methylation.

The N660/66-mer was 5′-labeled using T4 polynucleotide kinase and [³²P]-ATP according to the manufacturer's protocol (U.S. Biochemical). Unreacted [³²P]-ATP and T4 polynucleotide kinase were separated from labeled duplex DNA by eluting the DNA through a 1-mL G-25 gel filtration column. Methylation assays were performed using 250 nM CcrM, 2 pM 5′-labeled N660/66-mer, 6 pM [³H]-SAM in the appropriate reaction buffer at 30° C. 5 pL of reaction was quenched with 500 pL 10% perchloric acid, 200 pL saturated sodium pyrophosphate, and 20 pL single-stranded DNA at times varying from 15 seconds to 20 minutes. These reactions were placed on ice for at least 30 minutes, and then were subjected to the filter binding assay monitoring [³H]-CH₃ incorporation from [³H]-SAM into duplex DNA as described in Example 10.

Concomitantly, 20 pL reaction aliquots were quenched by either heat denaturation of CcrM or by the addition of 50 pL phenol/chloroform at times varying from 15 seconds to 20 minutes. The quenched reactions were then subjected to HindIII digestion. Typically, these reactions consisted of 10 pL of the quenched DNA in a 20 pL reaction with the appropriate reaction buffer and 1 pL of HindIII. After three hours of HindIII digestion at 37° C., 10 pL of this reaction was quenched with 10 pL of gel loading dye. DNA fragments were then resolved by 16% denaturing gel electrophoresis followed by PhosphorImaging to identify cleavage patterns.

Results from the [³H]-SAM assay indicated that two mole equivalents of [³H]-CH₃ were incorporated into the N660166-mer after 20 minutes. By direct contrast, only one mole equivalent of [³H]-CH₃ was incorporated into the N623/30-mer or N645/50-mer after 20 minutes under identical conditions. Results from the HindIII digestion assay reveal fully protected DNA substrate (N660/66-mer) after 20 minutes, indicating that DNA had been methylated at both GANTC sites. Furthermore, no intermediate products were obtained, i.e., methylation at a single GANTC site, indicating that under the assay conditions used the enzyme processively methylated both GANTC sites on the same DNA substrate. Approximately 250 nM of processively methylated DNA was detected after PhosphorImaging quantitation, consistent with results from the tritium incorporation assay.

The disclosures in this application of all articles and references, including patents, are incorporated herein by reference.

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLE 1

Preparation of Solution Phase Chemistry Libraries

Solution phase combinatorial libraries as described above were prepared in a 96-well microtitre plate as follows.

To each well in columns 1-7 was added K₂CO₃ (7-10 mg) followed by DMF (140 μL) and the 6-chloropurine (30 μL of 0.5M solution in DMF). To each row was added a halide (15 μL of a 1M solution in DMF) selected from the list of halides disclosed herein.

To each well in columns 8-10 was added K₂CO₃ (3-5 mg) followed by DMF (180 μL) and the 6-chloropurine (10 μL of 0.5M solution in DMF). To each row was added a halide (5 μL of a 1M solution in DMF) selected from the list of halides disclosed herein.

The microtitre plate was heated to 45° C. and reacted overnight. Reactions were cooled to room temperature and the second synthetic reaction performed as follows.

To each well in columns 1-7 was added 3 different amines (5 μL each of a 1M solution in DMF) selected from the list of amines disclosed herein.

To each well in columns 8-10 was added one amine (5 μL each of a 1M solution in DMF) selected from the list of amines disclosed herein.

The microtitre plate was heated to 85° C. and reacted overnight. Reactions were cooled to room temperature, each well was collected separately and the final volume of the solution was adjusted to 300 μL (replacing solvent lost to evaporation).

The compounds produced in these reactions were then tested using the in vivo bacterial growth assays disclosed herein.

### EXAMPLE 2

Preparation of Adenine DNA Methyltransferase Inhibitors

The combinatorial libraries of the invention were screened for adenine DNA methyltransferase inhibitory activity as described above. A compound displaying methyltransferase inhibiting activity and having the C6 amino group covalently linked to diphenylborinic acid ethanolamine ester was used as the base compound for preparing related analogues according to the following reaction scheme: 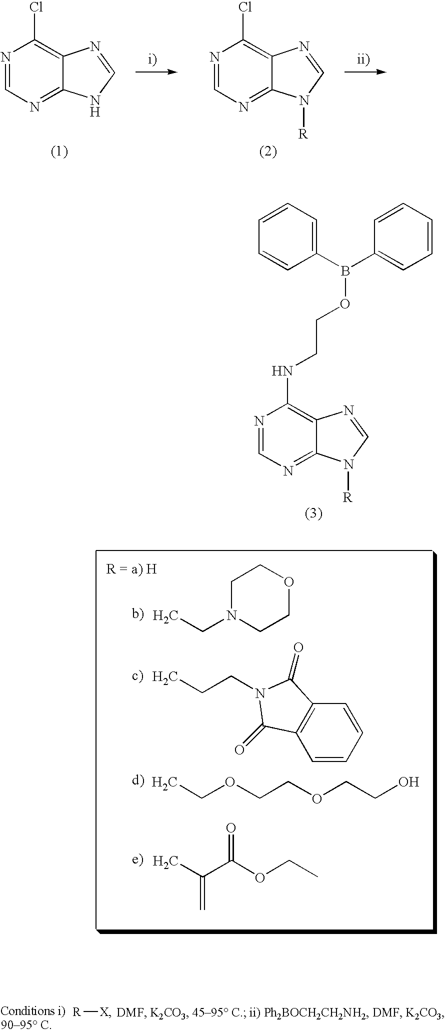
Specifically, 6-chloropurine (1) was dissolved in dry DMF (about 0.3 mmol/mL) under argon at room temperature. Potassium carbonate (K₂CO₃; 2-3 equivalents) was added, followed by one equivalent of the alkyl halide (R—X). The reaction was heated to either 45° C. or 95° C. (if the halide does not react at the lower temperature) and stirred for 18 hours. After this time, thin layer chromatography was performed (using 2%-5% methanol in dichloromethane as solvent) and showed little or no starting material remaining in the reaction mixture. The reaction was cooled to room temperature and the solid removed by filtration and washed with dry dimethylformamide (DMF). The filtrate was obtained and any remaining DMF was removed in vacuo to give the crude product as an oil. The product was purified by column chromatography on silica gel (using 2%-5% methanol in dichloromethane as solvent). The N-9 regioisomer was eluted as a pure fraction before a combination of the N-9 and N-7 regioisomers that eluted as a mixture. The fractions containing the pure N-9 isomer were combined and the solvent was removed in vacuo to give the intermediate product 2b-2e as a solid or an oil that solidified on standing.

The final compounds were prepared as follows. 6-chloropurine (1) or 9-alkyl-6-chloropurine (2b-2e) was dissolved in dry DMF (0.03-0.5 mmol/mL) under argon at room temperature. Potassium carbonate (K₂CO₃; 1.5-2 equivalents) was added followed by one equivalent of diphenylborinic acid ethanolamine ester. The reaction was heated to 90-95° C. and stirred for 18 hours. The mixture was allowed to cool to room temperature and the solid was removed by filtration as described above. The structure of these compounds was confirmed using ¹H-NMR, ¹³C-NMR, and two-dimensional NMR spectroscopic methods such as HMQC and HMBC.

Alternatively, 6-chloropurine (1) or N-9 alkyl-6-chloropurine (2b-2e) was dissolved in 1-butanol (0.1 mmol/mL) under argon at room temperature. Three equivalents of diisopropylethylamine were added, followed by the addition of 1.2 equivalents of alkyl halide. This reaction mixture was heated to 110° C. and stirred for 18 hours. The reaction was then cooled to room temperature and the solvent was removed in vacuo. The residue was purified by column chromatography on silica gel (using a solution of 2% to 5% methanol in dichloromethane in solvent). The product fractions were collected and the solvent was removed in vacuo to leave a white solid.

It is known in the art that the structure of the staring material, diphenylborinic acid ethanolamine ester, is cyclic and the boron is tetrahedral. Thus, cyclic and linear analogues of the adenine DNA methyltransferase inhibiting compounds of the invention may be advantageous for developing additional inhibitory compounds.

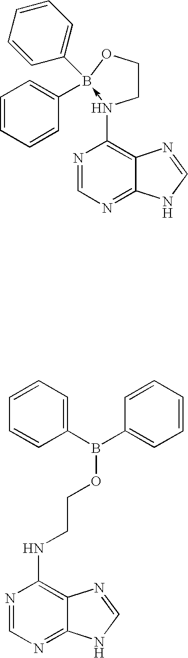

For in vivo assays, the unpurified preparation, which contained residual DMF or dimethylsulfoxide (DMSO), was diluted to 16.7 mM and used directly as a crude mixture.

In vivo assays of bacterial cell growth inhibition were performed essentially as described above using a variety of bacterial species. The compounds (3a) through (3e) showed the following results in these assays:

Caulobacter cresentus

Compound 3a —IC₅₀<25 μM
Compound 3b —IC₅₀<25 μM
Compound 3c —IC₅₀<25 μM
Compound 3d —IC₅₀<25 μM
Compound 3e —IC₅₀<25 μM
   Brucella abortus
Compound 3a—Almost complete cell death after 12 hours—100 μM
Compound 3b—Complete cell death after 12 hours—100 μM
Compound 3c—Cell growth inhibited from start —100 μM
Compound 3d—Cell growth inhibited from start —100 μM
Compound 3e—Cell growth inhibited from start —100 μM
   Helicobacter pylori
Compound 3a—IC₅₀<25 μM
Compound 3b—IC₅₀<25 μM
Compound 3c—IC₅₀ between 25-100 μM
Compound 3d—IC₅₀ between 25-100 μM
Compound 3e—IC₅₀=25 μM
   Agrobacterium tumefaciens
Compound 3a—IC₅₀>100 μM
Compound 3b—IC₅₀=25 μM
Compound 3c—IC₅₀=25 μM
Compound 3d—IC₅₀<<25 μM Compound 3e—IC₅₀<<25 μM
   Bacillus subtilis
Compound 3a—IC₅₀ between 10-50 μM
Compound 3b—IC₅₀ between 1-10 μM
Compound 3c—IC₅₀ between 1-10M
Compound 3d—IC₅₀ between 10-50 μM
Compound 3e—not tested

In addition, the following results were obtained using in vitro adenine DNA methyltransferase inhibition assays:

CcrM

Compound 3a—Complete inhibition at 100 μM
Compound 3b—Complete inhibition at 100 ∥M
Compound 3c—Complete inhibition at 100 μM
Compound 3d—Complete inhibition at 100 μM
Compound 3e—Complete inhibition at 100 μM
   dam Methylase (E. coli)
Compound 3a—Complete inhibition at 1001M
Compound 3b—Complete inhibition at 100 μM
Compound 3c—Complete inhibition at 100 μM
Compound 3d—Complete inhibition at 100 μM
Compound 3e—Complete inhibition at 100 μM
   dcm Methyltransferase (Hhal)
Compound 3a—No inhibition at all at 500 μM
Compound 3b—No inhibition at all at 500 μM
Compound 3c—No inhibition at all at 500 μM
Compound 3d—No inhibition at all at 500 μM
Compound 3e—No inhibition at all at 500 μM

These results demonstrate that compounds (3a) through (3e) are adenine-specific DNA methyltransferases with no detectable dcm crossreactivity.

### EXAMPLE 3

Preparation of Adenine DNA Methyltransferase Inhibitors

Additional adenine DNA methyltransferase inhibitors were developed by optimization of leads found during screening of the combinatorial libraries described above.

1. 6-(2-diphenylmethylcyclopentylamino)purine (Compound 73)

6-chloropurine was combined with S-(−)-2-(diphenylmethyl)-pyrrolidine in n-butanol with two equivalents of diisopropylethylamine (N(iPr)₂Et). The reaction was heated to 105° C. and allowed to react for 24 h. Solvent was removed from the reaction mixture in vacuo and the crude product purified by silica gel chromatography.

2. 6-(2-diphenylhydroxymethylcyclopentylamino)purine (Compound 71)

6-chloropurine was combined with R-(+)-α,α-diphenyl-2-pyrrolidinemethanol in n-butanol with two equivalents of N(iPr)₂Et. The reaction was heated to 95° C. and allowed to react for 24 h. Solvent was removed from the reaction mixture in vacuo and the crude product purified by silica gel chromatography.

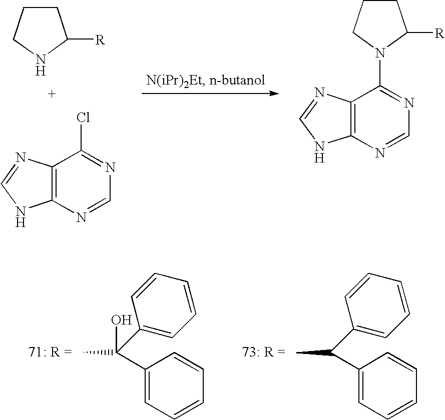

3. 2-diphenylpyrrole (Compound 76) 2-pyrrolidinone was combined with dimethylsulphate in benzene and refluxed for 3 h. After purification including distillation, the resulting 2-methylimino ester pyrrolidine was combined with excess lithium phenoxide (PhLi) in dry ether at room temperature for 18 h to yield the title compound.

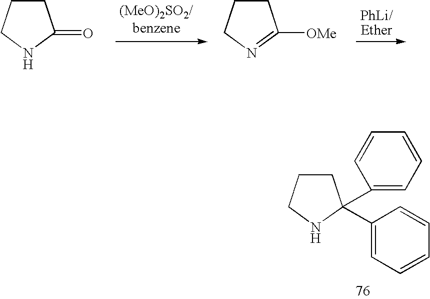

4. 6-amino-4(2-diphenylborinic ester) ethylamino pyrimidine (Compound III168)

4-amino-6-hydroxy-2-thiopyrimidine was treated with Raney nickel (RaNi) in water and ammonia and heated to reflux for 2 h. Purification afforded the 4-amino-6-hydroxypyrimidine, which was combined with phosphorus oxychloride and N,N-diethylaniline and heated at reflux for 4 h to give 4-amino-6-chloropyrimidine. This product was combined with diphenylborinic acid ethanolamine ester in toluene with diisopropylethylamine and heated to reflux overnight to produce the title compound.

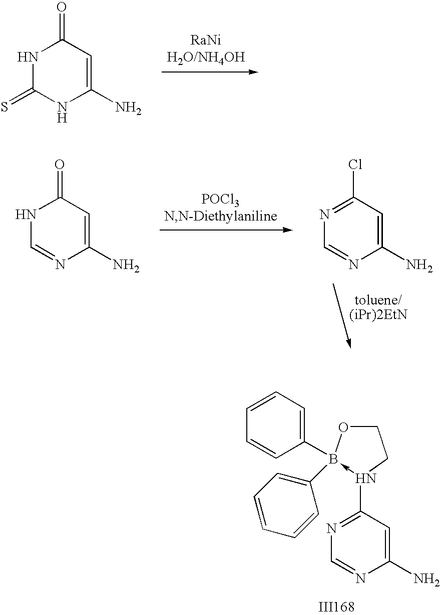

5. 4-amino-5(2-diphenylborinic ester ethyliminoester)imidazole (Compound III170)

4-amino-5-imidazolecarboxamide hydrochloride was heated to reflux in phosphorus oxychloride for 3.5 hs and after purification gave 4-amino-5-nitrile imidazole. This was resuspended in ethanol saturated with HCl overnight and purification gave 4-amino-5-ethylimino ester imidazole hydrochloride. This was combined with diphenylborinic acid ethanolamine ester in tetrahydrofuran (THF) overnight to yield the title compound.

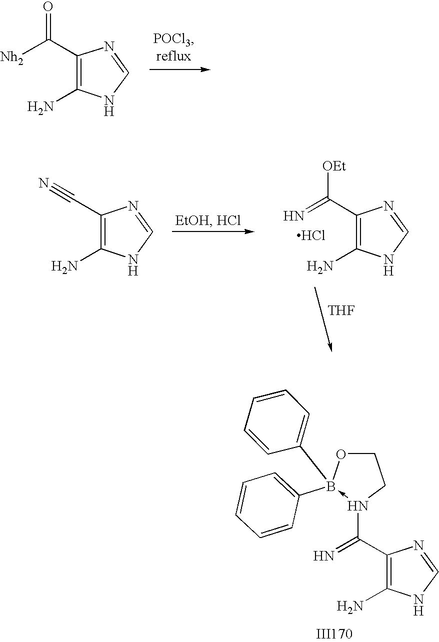

### EXAMPLE 4

Second Generation Libraries

Two “second generation libraries were prepared based on the compounds:

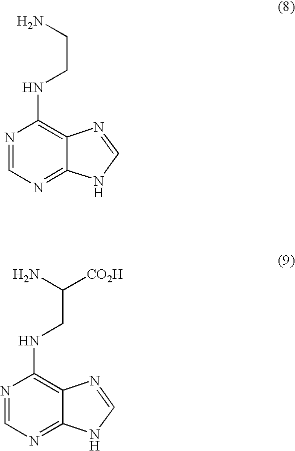

The first second generation library (“library A”) was constructed as shown below from parent compound 78. Compound 78 was combined with one equivalent of aldehyde (or ketone) in methanol at 25° C. in a heater-shaker.

Reactions were set up in duplicate. After reaction for one hour, BH₃-resin was added and the mixture was allowed to react overnight. To one set of the reaction was added a second equivalent of one of the following aldehydes:
cyclohexanecarboxaldehyde;
3-furaldehyde;
1-methyl-2-pyrrolecarboxaldehyde;
hydrocinnamaldehyde;
4-pyridine carboxaldehyde;
2-phenylproprionaldehyde;
phenylacetaldehyde;
m-anisaldehyde;
heptaldehyde;
3-nitrobenzaldehyde;
3-phenylbutyraldehyde;
3-pyridylacetaldehyde N-oxide;
ethyl leveulinate;
ethyl-2-ethylacetoacetone;
ethyl-4-acetylbutyrate;
ethyl proprionylacetate;
ethyl 2-benzylacetoacetone;
1-phenyl-2-pentanone;
1-carbethoxy-4-piperidone;
N-acetonylphthalimide;
2-fluorophenylacetone;
4-(3-oxobutyl)phenylacetate.

The entire plate was then allowed to react for a further 24 hrs. The compounds generated are either monoalkylated, with R=H, R′=alkyl, aryl or dialkylated with R=R′=alkyl or aryl.

The other second-generation library (“library B”) was constructed by reacting the parent compound III142 with the amines used to construct the N6 library and set forth above in the presence of trimethyl aluminum (AlMe₃) in dichloromethane at 50° C. overnight. The solvent was removed by evaporation, and the residue was dissolved in acetonitrile and treated with trimethylsilyl iodide overnight. Reactions were worked up by adding methanol, evaporating the solvents, partitioning the residue between ether and water/acetic acid (7:3) and extracting the product into the aqueous layer.

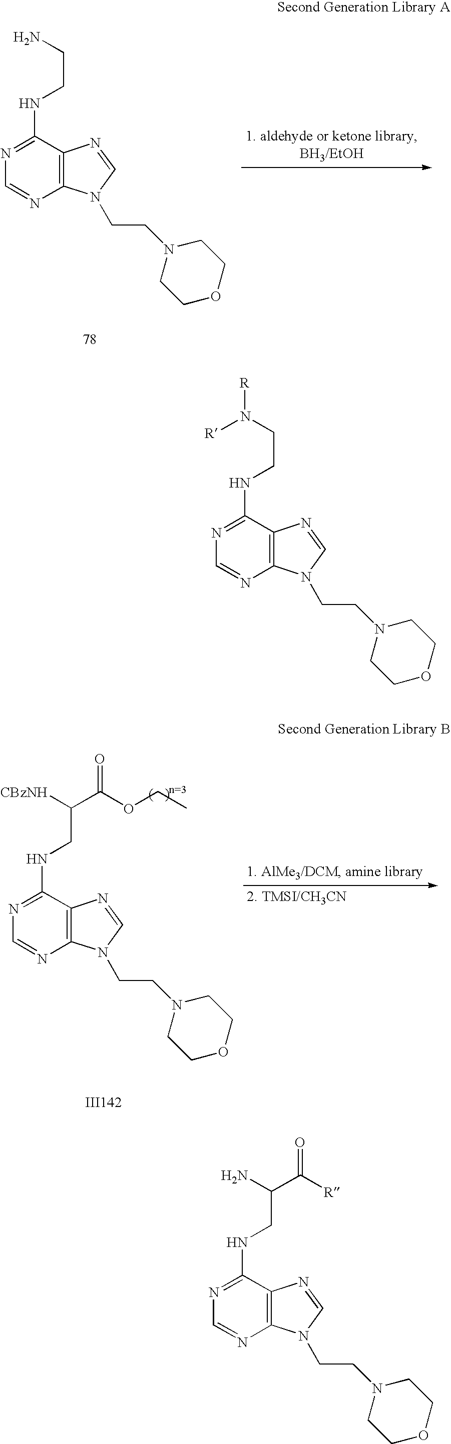

### EXAMPLE 5

Compounds based on Diphenyl Borinic Esters

Based on the results disclosed above, one common component of several of the adenine DNA methyltransferase inhibitors of the invention is diphenyl borinic ester. Accordingly, several additional compounds based on this ester were prepared as follows. These compounds have the following structures: 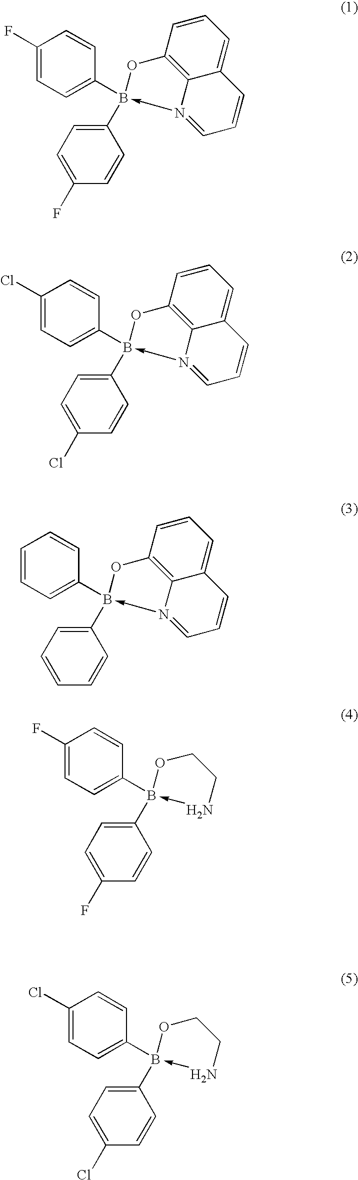
The general synthesis of these compounds is shown in Reaction Scheme 7.

1. Synthesis of Borinic Acids (Compound 8).

Dichloroborane dimethyl sulfide complex (0.5-2 mL) was dissolved in either tetrahydrofuran or diethyl ether under argon and cooled to −78° C. he appropriate phenyl Grignard reagent (2 molar equivalents), in tetrahydrofuran, diethyl ether, cyclohexane or mixtures of these solvents, was added dropwise to the cold reaction. The reaction was allowed to warm to room temperature and stirred overnight. Diethyl ether was added to the reaction and the reaction was hydrolyzed by the slow addition of 1N hydrochloric acid. The layers were separated and the organic layer was washed with saturated aqueous NaCl. The organic layer was dried over magnesium sulfate (MgSO₄), filtered and the solvent was removed in vacuo to give the crude product as a clear oil. This crude preparation of the title compound was used directly in the next stage of the synthesis 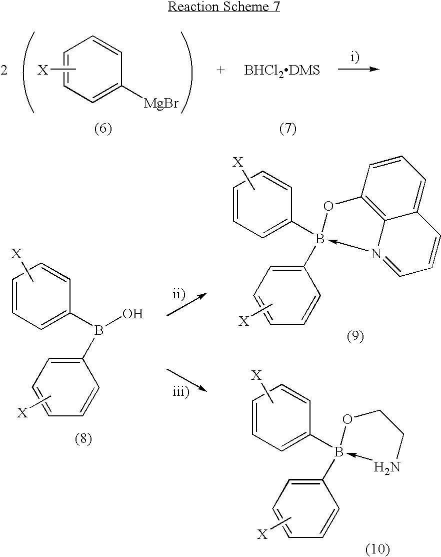
where the reaction conditions are:
i) tetrahydrofuran (THF) or ethyl ether (Et₂O), −78° C. to room temperature overnight;
ii) EtOH, 8-hydroxyquinoline, room temperature;
iii) EtOH, 2-aminoethanol, room temperature.
   X can represent up to 5 substituents on each phenyl group, which can be independently hydrogen, lower alkyl, aryl or substituted aryl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from oxygen and nitrogen, and where any member of the alkyl, aryl or cycloalkyl group is optionally substituted with halogen, lower alkyl or lower alkoxy, aryl or substituted aryl, halide, nitro, nitroso, aldehyde, carboxylic acid, esters, amides, or sulfates.

2. Synthesis of Borinic Acid 8-hydroxyquinoline Esters (Compounds 9).

The crude borinic acid (8) was dissolved in 0.05-5 mL ethanol and was treated with 1-2 equivalents of 1M 8-hydroxyquinoline in ethanol. The product either precipitated from the solution or the solution was concentrated and left to crystallize, once a solid had formed; the product was collected by filtration and washed with ethanol.

3. Synthesis of Borinic Acid Ethanolamine Esters (Compounds 10).

The crude borinic acid (8) was dissolved in 0.05-5 mL ethanol and treated with 1-2 equivalents of 1M ethanolamine in ethanol. The product either precipitated from the solution or the solution was concentrated and left to crystallize; once a solid had formed, the product was collected by filtration and washed with ethanol.

The compounds (1)-(5) prepared as described above were tested using the in vivo assays of the invention using Caulobacter cresentus, and compounds (1), (2), (4) and (5) were tested for cell growth inhibition against Bacillus subtilis. The IC₅₀ values are shown in Table II.

TABLE II

| | Compound (1) | Compound (2) | Compound (3) | Compound (4) | Compound (5) |
|---|---|---|---|---|---|
| Caulobacter cresentus | 23 μM | 1.6 μM | >100 μ□M | 8.5 μM | 1.7 μM |
| Bacillus subtilis | 1.4 μM | 7 μM | Not tested | 3.4 μM | 3.2 μM |

These compounds have advantageous physical properties, and are isolated as pure, stable solids that are amenable to large-scale production. Additional specific embodiments of adenine DNA methyltransferase inhibitors of the invention includes related compounds having these additional features:
1) Analogues with various substituents on the phenyl rings in any, or combination of, the ortho-, meta- and para-positions, including fused rings and substituted fused rings;
2) Analogues having aromatic heterocycles of various ring sizes, substituted heterocycles, fused heterocycles and substituted fused heterocycles in place of one or both phenyl groups;
3) Analogues having two non-identical aromatic rings bound to the boron atom, using combinations of the aromatic systems described in 1) and 2) above;
4) Analogues prepared using quinolines (9) containing various substituents in any possible position or structural analogues including fused heteroaromatic rings containing one or more heteroatom in any possible position or fused heteroaromatic rings containing one or more heteroatom in any possible position and containing various substituents in any possible position; and
5) Analogues having substitutions on either, or both of, the C-1 and C-2 positions of the ethylene group of the 2-aminoethanol of (10).

### EXAMPLE 6

Compounds based on Diphenyl Borinic Esters

Several compounds based on diphenyl borinic ester were prepared as follows. The general synthesis of these compounds is shown in Reaction Scheme 8. 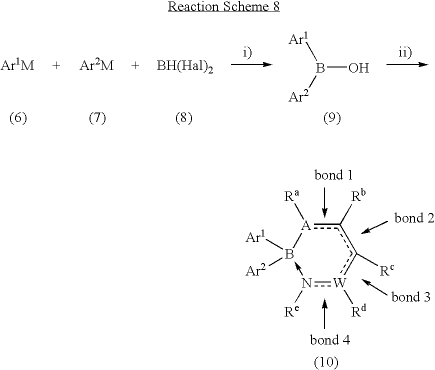
where the reaction conditions are:
i) tetrahydrofuran (THF) or ethyl ether (Et₂O), −78° C. to room temperature overnight;
ii) EtOH, room temperature, boron coordinating agent;
   and where
M=MgBr, Li
Hal=Cl, Br
A=O, N, S
W=Cₚ where p=0, 1
R^{a}, R^{b}, R^{c}, R^{d}, and R^{e} are the same or different and are independently hydrogen, halogen, nitro, nitroso, lower alkyl, aryl or substituted aryl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from sulfur, oxygen and nitrogen, and where any member of the alkyl, aryl or cycloalkyl group is optionally substituted with halogen, lower alkyl or lower alkoxy, aryl or substituted aryl, halogen, nitro, nitroso, aldehyde, carboxylic acid, amide, ester, or sulfate, or wherein R^{a}, R^{b}, R^{c}, R^{d}, and R^{e} may be connected by aromatic, aliphatic, heteroaromatic, heteroaliphatic ring structures or substituted embodiments thereof; where R^{a} is absent when A is O or S and R^{d} is absent when p=0
bond 1, bond 2, bond 3 and bond 4 are independently a single or a double bond, and when A=O, S, bond 1 is a single bond and when A=N, bond 1 is a single or a double bond and
X can represent up to 5 substituents on each phenyl group, which can be independently hydrogen, lower alkyl, aryl or substituted aryl, lower alkoxy, lower alkoxyalkyl, or cycloalkyl or cycloalkyl alkoxy, where each cycloalkyl group has from 3-7 members, where up to two of the cycloalkyl members are optionally hetero atoms selected from oxygen and nitrogen, and where any member of the alkyl, aryl or cycloalkyl group is optionally substituted with halogen, lower alkyl or lower alkoxy, aryl or substituted aryl, halide, nitro, nitroso, aldehyde, carboxylic acid, esters, amides, or sulfates.

Preferred compounds are identified herein based on the identity of the substituents Ar¹=Ar² where: 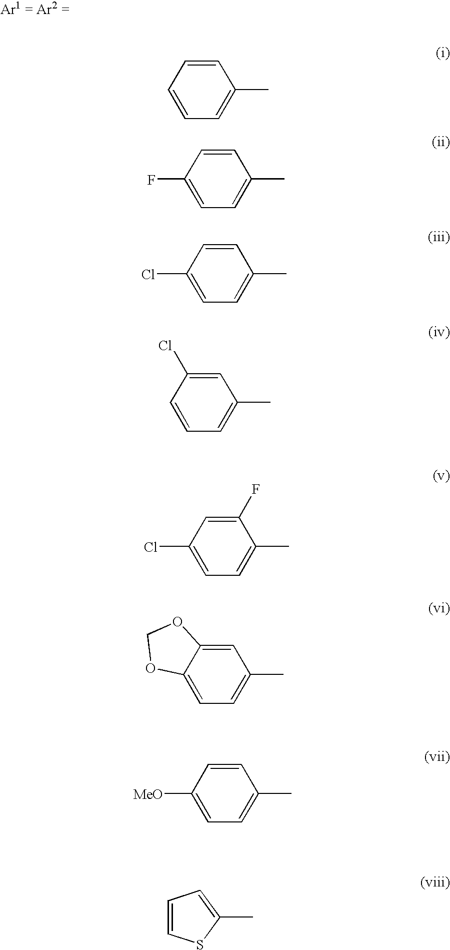
are in combination with any one of the following: 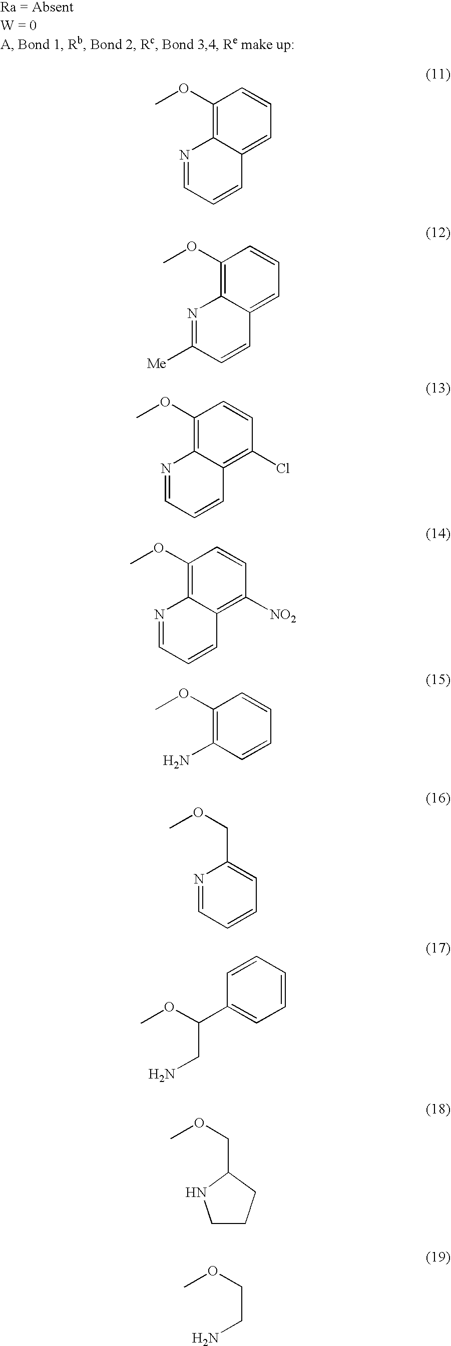
General Experimental Protocols

Chemicals were purchased from Acros Organics and Aldrich Chemical Company (Milwaukee, Wis.) and were used without further purification. Tetrahydrofuran was dried by distillation from sodium and benzophenone; diethyl ether was dried over sodium and distilled; all other solvents used were the highest available grade and used without further purification.

Reactions were performed as set forth in detail below. Reaction products were analyzed by ¹H-NMR spectra recorded on a Bruker Avance 400 (400 MHz) and Bruker AMX360 (360 MHz). MALDI mass spectra were obtained using a Perspective Biosystems Voyager-DE STR, FAB mass spectra were obtained using a Kratos Analytical MS-50 TC, and APCI mass spectra were recorded on a Perspective Biosystems Mariner. Microanalyses were recorded by Atlantic Microlab Inc. (Norcross, Ga. 30091). Analytical thin layer chromatography (tic) was performed with Whatman silica gel aluminum backed plates of thickness 250 μm and fluorescent at 254 nm, and by using the solvent systems indicated. Flash column chromatography was performed with Selecto Scientific silica gel, 32-64 μm particle size. Melting points were obtained using a MeI-Temp II melting point apparatus with a Fluke K1 K/J type thermocouple digital thermometer and are uncorrected. Purity was determined by HPLC using a betabasic-18 (4.6 mm×15 cm) column from Keystone Scientific Inc. and product eluted using a linear gradient of 0 to 40% acetonitrile in 10 mM triethyl ammonium acetate over 20 mins.

Caulobacter crescentus strain CB15N was a gift from Prof. Lucille Shapiro, Department of Developmental Biology, Stanford University, Stanford, Calif. 94305, USA. Bacillus subtilis (ATCC #33234) was obtained from ATCC, Manassas, Va.

General Methods for the Synthesis of Diaryl Borinic Acids (9)

Method A: Dichloroborane dimethyl sulfide complex or dibromoborane dimethyl sulfide (1 molar equivalent) was added to either tetrahydrofuran (0.2 mmol/mL) or diethyl ether (0.2 mmol/mL) under argon and cooled to −78° C. The aryl magnesium bromide (2 molar equivalents), in tetrahydrofuran, diethyl ether, cyclohexane, toluene or mixtures of these solvents, was added dropwise to the cold reaction mixture. The reaction was allowed to warm to room temperature and stirred overnight. The solvents were removed in vacuo and the residue was dissolved in diethyl ether. The reaction was stirred rapidly and hydrolyzed by the slow addition of 1N hydrochloric acid. Stirring was discontinued, the layers were separated and the organic layer was washed with saturated aqueous NaCl. The organic layer was dried over magnesium sulfate (MgSO₄), filtered and the solvent removed in vacuo to give the crude product as an oil. This oil was dissolved in ethanol to an estimated concentration of 1M and divided into portions to be used in the subsequent precipitation with the various complexing agents.

Method B: Dichloroborane dimethyl sulfide complex or dibromoborane dimethyl sulfide complex (1 molar equivalent) was added to either tetrahydrofuran (2 mmol/mL) or diethyl ether (2 mmol/mL) under argon and cooled to −78° C. The aryl lithium (2 molar equivalents), in tetrahydrofuran, diethyl ether, cyclohexane or mixtures of these solvents, was added dropwise to the cold reaction mixture. The reaction was allowed to warm to room temperature and stirred overnight. The solvents were removed in vacuo and the residue was dissolved in diethyl ether. The reaction mixture was stirred rapidly and hydrolyzed by the slow addition of 1N hydrochloric acid. Stirring was discontinued, the layers were separated and the organic layer was washed with saturated aqueous NaCl. The organic layer was dried over magnesium sulfate (MgSO₄), filtered and the solvent removed in vacuo to give the crude product as an oil. This oil was dissolved in ethanol to an estimated concentration of 1M and divided into portions to be used in the subsequent precipitation with the various complexing agents.

Products are identified herein as a combination of the constituents identified above. Thus, di-(4-fluorophenyl)borinic acid 8-hydroxyquinoline ester is identified as 11ii, indicating that Ar¹=Ar² and are each 4-fluorophenyl (substituent 11 above), and R^{a} is absent, p=0, and A, bond 1, R^{b}, bond 2, R^{c}, bond 3, bond 4 and R^{e} make up hydroxyquinoline (substituent ii above).

Di-(4-fluorophenyl)borinic acid 8-hydroxyguinoline ester (11ii): Di-(4-fluorophenyl)borinic acid was prepared using method A and was treated with 8-hydroxyquinoline (0.5M solution in ethanol). A yellow precipitate formed immediately and was collected by filtration and washed with ethanol. The product had the following properties upon analysis: mp 166-167° C.; ¹H-NMR (360 MHz, C²HCl_{3,}): δ 8.53 (d, J=5.1 Hz, 1H), 8.45 (d, J=8.2 Hz, 1H), 7.70 (dd, J=8.2, 7.7 Hz, 1H), 7.66 (dd, J=8.2, 5.1 Hz, 1H), 7.39 (dd, J=8.8, 6.7 Hz, 4H), 7.30 (d, J=8.2 Hz, 1H), 7.20 (d, J=7.7 Hz, 1H), 6.97 (t, J=8.8 Hz, 4H); MS (+ve APCI) m/z 345 ([M+H]⁺, ¹⁰B), 346 ([M+H]⁺, ¹¹B), 368 ([M+Na]⁺, ¹¹B); Anal. (C₂₁H₁₄NOBF₂) C, H, N.

Di-(4-chlorophenyl)borinic acid 8-hydroxyquinoline ester (11iii): Di-(4-chlorophenylborinic acid was prepared using method A and was treated with 8-hydroxyquinoline (0.5M solution in ethanol). A yellow precipitate formed immediately and was collected by filtration and washed with ethanol. The product had the following properties upon analysis: mp 192-194° C.; ¹H-NMR (360 MHz, C²HCl_{3,}): δ 8.49 (dd, J=4.6, 1.0 Hz, 1H), 8.43 (dd, J=8.2, 1.0 Hz, 1H), 7.70 (dd, J=8.5, 7.7 Hz, 1H), 7.66 (dd, J=8.2, 4.6 Hz, 1H), 7.31 (d, J=8.2 Hz, 4H), 7.26 (d, J=8.5 Hz, 1H), 7.21 (d, J=8.2 Hz, 4H), 7.17 (d, J=7.7 Hz, 1H); MS (+ve APCI) m/z 377 ([M+H]⁺, ¹⁰B, ³⁵Cl, ³⁵Cl), 378 ([M+H]⁺, ¹¹B, ³⁵Cl, ³⁵Cl), 379 ([M+H]⁺, ¹⁰B, ³⁵Cl, ³⁷Cl), 380 ([M+H]⁺, ¹¹B, ³⁵Cl, ³⁷Cl), 381 ([M+H]⁺, ¹⁰B, ³⁷Cl, ³⁷Cl), 382 ([M+H]⁺, ¹¹B, ³⁷Cl, ³⁷Cl); Anal. (C₂₁H₁₄NOBCl₂) C, H, N.

Di-(3-chlorophenyl)borinic acid 8-hydroxyguinoline ester (11iv): Di-(3-chlorophenyl)borinic acid was formed using method A and was treated with 8-hydroxyquinoline (1.0 M in ethanol). The solvent was removed in vacuo and the residue was purified by flash chromatography on silica gel using CH₂Cl₂/hexane (1:1) to elute the product. The solvent was evaporated and the product crystallized upon the addition of ethanol. The solid was collected by filtration and washed with cold ethanol to yield the title product as a yellow solid having the following properties: mp 144-145° C.; ¹H-NMR (360 MHz, C₂H₃O²H): 8.83 (d, J=5.0 Hz, 1H), 8.63 (d, J=8.2 Hz, 1H), 7.78 (dd, J=8.6, 5.0 Hz, 1H), 7.67 (t, J 8.2, 1H), 7.36 (d, J=8.2, 1H), 7.26-7.09 (m, 9H); MS (+ve ESI) 377 ([M+H]⁺, ¹⁰B, ³⁵Cl, ³⁵Cl), 378 ([M+H]⁺, ¹¹B, ³⁵Cl, ³⁵Cl), 379 ([M+H]⁺, ¹⁰B, ³⁵Cl, ³⁷Cl), 380 ([M+H]⁺, ¹¹B, ³⁵Cl, ³⁷Cl), 381 ([M+H]⁺, ¹⁰B, ³⁷Cl, ³⁷Cl), 382 ([M+H]⁺, ¹¹B, ³⁷Cl, ³⁷Cl); Anal. (C₂₁H₁₄NOBCl₂) C, H, N.

Di-(4-chloro-2-fluorophenyl)borinic acid 8-hydroxyquinoline ester (11v): Di-(4-chloro-2-fluorophenyl)borinic acid was formed using method A and was treated with 8-hydroxyquinoline (0.5M in ethanol). The residue was purified by flash chromatography on silica gel using hexane/ethyl acetate (3:1). The solvent was evaporated and the product crystallized upon the addition of ethanol. The solid was collected by filtration and washed with cold ethanol to yield the title product as a yellow solid having the following properties: mp 135° C.; ¹H-NMR (360 MHz, C²H₃₀²H): 8.82 (d, J=5.0 Hz, 1H), 8.61 (d, J=8.2 Hz, 1H), 7.75 (dd, J=8.2, 5.0 Hz, 1H), 7.63 (t, J=8.2 Hz, 1H), 7.35 (d, J=8.2 Hz, 1H), 7.27 (t, J=7.7 Hz, 2H), 7.13-6.90 (m, 5H); MS (+ve APCI) 413 ([M+H]⁺, ¹⁰B, ³⁵Cl, ³⁵Cl), 414 ([M+H]⁺, ¹¹B, ³⁵Cl, ³⁵Cl), 415 ([M+H]⁺, ¹⁰B, ³⁵Cl, ³⁷Cl), 416 ([M+H]⁺, ¹¹B, ³⁵Cl, ³⁷Cl), 417 ([M+H]⁺, ¹⁰B, ³⁷Cl, ³⁷Cl), 418 ([M+H]⁺, ¹¹B, ³⁷Cl, ³⁷Cl); Anal. calcd for C₂₁H₁₂NOBF₂Cl₂(0.5H₂O): C, 59.62; H, 3.10; N, 3.31; found: C, 59.74; H, 3.03; N, 3.18.

Di-(3,4-methylenedioxyphenyl)borinic acid 8-hydroxyquinoline ester (11 vi): Di(3,4-methylenedioxyphenyl)borinic acid was formed using method A and was treated with 8-hydroxyquinoline (0.5M in ethanol). The solution was allowed to stand overnight to crystallize. The solid was collected by filtration and washed with cold ethanol to yield the product as a yellow solid having the following properties: mp 174-176° C.; ¹H-NMR (400 MHz, C²HCl₃): 8.52 (d, J=4.9 Hz, 1H), 8.41 (d, J=8.2 Hz, 1H), 7.66 (t, J=7.9 Hz, 1H), 7.63 (dd, J=8.2, 5.0 Hz, 1H), 7.25 (d, 1H), 7.17 (d, J=7.7 Hz, 1H), 6.91 (s, 2H), 6.89 (d, J=8.5 Hz, 2H), 6.76 (d, J=8.2 Hz, 2H), 5.87 (s, 4H); MS (+ve APCI) m/z 397 (M⁺, ¹⁰B), 398 (M⁺, ¹¹B); Anal. (C₂₃H₁₆BNO₅) C, H, N.

Di-(4-methoxyphenyl)borinic acid 8-hydroxyquinoline ester (11vii): Di(4-methoxyphenyl) borinic acid was formed using method A and was treated with 8-hydroxyquinoline (0.5M in ethanol) causing the title product to precipitate from the solution. The solid was collected by filtration and washed with cold ethanol to yield the product as a yellow solid having the following properties: mp 222-224° C.; ¹H-NMR (400 MHz, C²HCl₃): 8.53 (d, J=5.0 Hz, 1H), 8.38 (d, J=8.5 Hz, 1H), 7.67 (t, J=8.0 Hz, 1H), 7.61 (dd, J=8.3, 5.0 Hz, 1H), 7.38 (d, J=8.5 Hz, 4H), 7.24 (d, J=8.2 Hz, 1H), 7.17 (d, J=7.7 Hz, 1H), 6.85 (d, J=8.6 Hz, 4H), 3.78 (s, 6H); MS (+ve MALDI, CHCA) m/z 369 ([M+H]⁺, ¹⁰B), 370 ([M+H]⁺, ¹¹B); Anal. (C₂₃H₂OBNO₃) C, H, N.

Di-(2-thienyl)borinic acid 8-hydroxyguinoline ester (11viii): Di-(2-thienyl)phenylborinic acid was prepared using method 2 and was treated with 8-hydroxyquinoline (0.5M solution in ethanol). A yellow precipitate formed upon standing and was collected by filtration and washed with ethanol. The isolated product had the following properties: mp 151-152° C.; ¹H-NMR (360 MHz, C²HCl₃): δ 8.58 (d, 1H, J=5.4 Hz), 8.39 (d, J=8.7 Hz, 1H), 7.62 (t, J=8.2, 1H), 7.60 (dd, J=8.7, 5.4 Hz, 1H), 7.39 (dd, J=4.6, 1.0 Hz, 4H), 7.26 (d, J=8.2 Hz, 1H), 7.22 (dd, J=3.4, 1.0 Hz, 2H), 7.20 (d, J=8.2 Hz, 1H), 7.08 (dd, J=4.6, 3.4 Hz, 2H); MS (+ve APCI) m/z 321 ([M+H]⁺, ¹⁰B), 322 ([M+H]⁺, ¹¹B); Anal. Calc for C₁₇H₁₂BONS₂.0.7(H₂O): C, 61.17; H, 4.05; N, 4.20; found C 61.15, H 4.09, N 4.25.

Di-(p-fluorophenyl)borinic acid 8-hydroxyquinaldine ester (12ii): Di-(p-fluorophenyl) borinic acid was formed using method A and was treated with 8-hydroxyquinaldine (0.5M solution in ethanol). The product was collected by filtration and washed with ethanol. The purified product had the following properties: mp 154-156° C.; ¹H-NMR (360 MHz, C²HCl₃): 8.21 (d, J=8.7 Hz, 1H) 7.47 (t, J=8.2 Hz, 1H), 7.28 (d, J=8.4, 1H), 7.22 (m, 4H), 7.12 (d, J=8.1 Hz, 1H), 6.98 (d, J=7.8 Hz, 1H), 6.85 (m, 4H) 2.39 (s, 3H); MS (+ve, APCI) m/z 359 ([M+H]⁺, ¹⁰B), 360 ([M+H]⁺, ¹¹B); Anal. (C₂₂H₁₆NBOF₂) C, H, N.

Di-(p-chlorophenyl)borinic acid 8-hydroxyguinaldine ester (12iii): Di-(p-chlorophenyl)-borinic acid was formed using method A and was treated with chloroquinaldine (0.5M solution in ethanol). The product was collected by filtration and washed with ethanol. The purified product had the following properties: mp 155-156° C.; ¹H-NMR (360 MHz, C²HCl₃): 8.21 (d, J=8.6 Hz, 1H), 7.46 (t, J=8.4 Hz, 1H), 7.27 (d, J=8.2 Hz, 1H), 7.18-7.11 (m, 9H), 6.97 (d, J=7.8 Hz, 1H) 2.38 (s, 3H); MS (+ve, APCI) m/z 392 (M⁺, ¹¹B, ³⁵Cl, ³⁵Cl), 394 (M⁺, ¹¹B, ³⁵C1, ³⁷Cl), 396 (M⁺, ¹¹B ³⁷Cl, ³⁷C1); Anal. (C₂₂H₁₆NBOCl₂) C, H, N.

Di-(4-methoxyphenyl)borinic acid 8-hydroxyquinaldine ester (12vii): Di-(4-methoxyphenyl) borinic acid was formed using method A and was treated with 8-hydroxyquinaldine (0.5M in ethanol). The solution was allowed to stand overnight to crystallize. The solid was collected by filtration and washed with cold ethanol to yield the title product as a yellow solid having the following properties: mp 150-151° C.; ¹H-NMR (400 MHz, C²HCl₃): 8.29 (d, J=8.4 Hz, 1H), 7.57 (t, J=8.0 Hz, 1H), 7.37 (d, J=8.4 Hz, 1H), 7.33 (d, J=8.5, 4H), 7.20 (d, J=8.2 Hz, 1H), 7.08 (d, J=7.7 Hz, 1H), 6.84 (d, J=8.5 Hz, 4H), 3.79 (s, 6H), 2.54 (s, 3H); MS (+ve MALDI, CHCA) m/z 383 ([M+H]⁺, ¹⁰B), 384 ([M+H]⁺, ¹¹B); Anal. (C₂₄H₂₂BNO₃) C, H, N.

Di-(p-fluorophenyl)borinic acid 5-chloro-8-hydroxyquinaline ester (13ii): Di-(p-fluorophenyl) borinic acid was formed using method A and was treated with 5-chloro-8-hydroxy quinaline (0.5M solution in ethanol). The product was collected by filtration and washed with ethanol. The purified product had the following properties: mp 143-145° C.; ¹H-NMR (360 MHz, C²HCl₃): 8.55 (d, J=8.3 Hz, 1H), 8.45 (d, J=5.0 Hz, 1H), 7.63 (m, 1H), 7.58 (d, J=8.15 Hz, 1H), 7.22 (m, 4H), 6.98 (d, J=8.14 Hz, 1H), 6.84 (m, 4H); MS (+ve, APCI) m/z 380 ([M+H]⁺, ¹¹B); Anal. (C₂₁H₁₃NBOClF₂) C, H, N.

Di-(p-chlorophenyl)borinic acid 5-chloro-8-hydroxyquinaline ester (13iii): Di-(p-chlorophenyl)borinic acid was formed using method A and was treated with 5-chloro-8-hydroxy quinaline (0.5M solution in ethanol). The product was collected by filtration and washed with ethanol. The purified product had the following properties: mp=154-156° C.; ¹H-NMR (360 MHz, C²HCl₃): 8.56 (d, J=Hz, 1H), 8.44 (d, J=Hz, 1H), 7.64 (m, 1H), 7.57 (d, J=Hz, 1H), 7.14 (m, 9H), 6.98 (d, 1H); MS (+ve, ESI) m/z 412 ([M+H]⁺, ¹⁰B, ³⁵Cl, ³⁵Cl, ³⁵Cl), 413 ([M+H]⁺, ¹¹B, ³⁵Cl, ³⁵Cl, ³⁵Cl); Anal. (C₂₁H₁₃NBOCl₂) C, H, N.

Di-(3,4-methylenedioxyphenyl)borinic acid 5-chloro-8-hydroxyquinoline ester (13vi): Di-(3,4-methylenedioxyphenyl)borinic acid was formed using method A and was treated with hot 5-chloro-8-hydroxyquinoline (0.5M in ethanol). The solution was allowed to stand overnight to crystallize. The solid was collected by filtration and washed with cold ethanol to yield the product as a yellow solid having the following properties: mp 212-213° C.; ¹H-NMR (360 MHz, C²HCl₃): 8.66 (d, J=8.4 Hz, 1H), 8.58 (d, J=4.9 Hz, 1H), 7.75 (dd, J=8.5, 5.1 Hz, 1H), 7.69 (d, J=7.3 Hz, 1H), 7.09 (d, J=8.3 Hz, 1H), 6.88 (s and d, overlapping, 4H), 6.76 (d, J=7.6 Hz, 2H), 5.88 (s, 4H); MS (+ve, APCI) m/z 432 ([M+H]⁺, ¹¹B, ³⁵Cl), 434 ([M+H]⁺, ¹¹B, ³⁷Cl); Anal. (C₂₃H₁₅BClNO₅) C, H, N.

Di-(4-methoxyphenyl)borinic acid 5-chloro-8-hydroxyquinoline ester (13vii): Di-(4-methoxyphenyl)borinic acid was formed using method A and was treated with hot 5-chloro-8-hydroxyquinoline (0.5 m in ethanol). The solution was allowed to stand overnight to crystallize. The solid was collected by filtration and washed with cold ethanol to yield the title product as a yellow solid having the following properties: mp 184-185° C.; ¹H-NMR (400 MHz, C²HCl₃): 8.66 (d, J=8.3 Hz, 1H), 8.59 (d, J=5.1 Hz, 1H), 7.73 (dd, J=8.4, 5.1 Hz, 1H), 7.69 (d, J=8.3 Hz, 1H), 7.34 (d, J=8.6 Hz, 4H), 7.09 (d, J=8.3 Hz, 1H), 6.84 (s, J=8.6 Hz, 4H), 3.78 (s, 6H); MS (+ve APCI) m/z 403 (M⁺, ¹¹B, ³⁵Cl), 405 (M⁺, ¹¹B, ³⁷Cl); Anal. (C₂₃H₁₉BClNO₃) C, H, N.

Di-(3,4-methylenedioxyphenyl)borinic acid 8-hydroxy-5-nitroquinoline ester (14vi): Di-(3,4-methylenedioxyphenyl)borinic acid was formed using method A and was treated with hot 8-hydroxy-5-nitroquinoline (0.5 m in ethanol). The solution was allowed to stand overnight to crystallize. The solid was collected by filtration and washed with cold ethanol to yield the product as a yellow solid having the following properties: mp 243-245° C.; ¹H-NMR (360 MHz, C²HCl₃): 9.69 (d, J=8.7 Hz, 1H), 8.90 (d, J=8.8 Hz, 1H), 8.68 (d, J=5.0 Hz, 1H), 7.97 (dd, J=8.7, 5.0 Hz, 1H), 7.17 (d, J=8.9 Hz, 1H), 6.84 (s, 2H), 6.82 (d, J=7.8 Hz, 2H), 6.77 (d, J=7.7 Hz, 2H), 5.90 (s, 4H); MS (+ve APCI) 442 (M⁺, ¹¹B); Anal. (C₂₃H₁₅BN₂O₇) C, H, N.

Diphenylborinic acid 2-aminophenol (15i): Diphenylborinic acid was prepared using method B and was treated with 2-aminophenol (1M solution in ethanol). A white precipitate formed upon standing and was collected by filtration and washed with ethanol. The purified product had the following properties: mp 179-181° C.; ¹H-NMR (360 MHz, C²H₃₀²H): δ 7.44 (m, 4H), 7.18 (m, 6H), 6.90 (dd, 1H), 6.76 (dd, 1H), 6.62 (m, 2H); MS (+ve,APCI) m/z 274 ([M+H]⁺, ¹¹B); Anal. (C₁₈H₁₆NOB): C, H, N.

Diphenylborinic acid pyridine-2-methanol (16i): Diphenylborinic acid was prepared using method B and was treated with pyridine-2-methanol (1M solution in ethanol). A white precipitate formed upon standing and was collected by filtration and washed with ethanol; mp 152-153° C.; ¹H-NMR (360 MHz, C²HCl₃): δ 8.40 (d, J=6.0 Hz, 1H), 7.98 (t, J=7.7 Hz, 1H), 7.54 (d, J=7.7 Hz, 1H), 7.30 (m, 4H), 7.23 (m, 7H); MS (+ve, APCI) m/z 274 ([M+H]⁺, ¹¹B); Anal. (C₁₈H₁₆NOB): C, H, N.

Diphenylborinic acid 2-amino-1-phenylpropanol (17i): Diphenylborinic acid was prepared using method B and was treated with 2-amino-1-phenylpropanol (1M solution in ethanol). A white precipitate formed upon standing and was collected by filtration and washed with ethanol. The purified product had the following properties: mp 200-201° C.; ¹H-NMR (360 MHz, C²H₃₀²H): δ 7.70-7.10 (m, 15H), 5.03 (dd, J=9.2, 6.4 Hz, 1H), 3.32 (dd, J=10.9, 6.4 Hz, 1H), 2.81 (dd, J=10.9, 9.2 Hz, 1H); MS (+ve, APCI) m/z 302 ([M+H]⁺, ¹¹B); Anal. (C₂₀H₂₀NOB) C, H, N.

Diphenylborinic acid (S)-(+)-pyrrolidine-2-methanol (18i): Diphenylborinic acid was prepared using method B and was treated with (S)-(+)-pyrrolidine-2-methanol (1M solution in ethanol). A white precipitate formed upon standing and was collected by filtration and washed with ethanol. The purified product had the following properties: mp 221-222° C.; ¹H-NMR (360 MHz, C²H₃₀²H): δ 7.44 (m, 4H), 7.13 (m, 6H), 3.94 (dd, J=6.6, 8.7 Hz, 1H), 3.77 (m, 1H), 3.68 (dd, J=6.6, 8.7 Hz, 1H), 2.86 (m, 1H), 2.60 (m, 1H), 2.14 (m, 1H), 1.93-1.65 (m, 3H); MS (+ve, APCI) m/z 266 ([M+H]⁺, ¹¹B); Anal. (C₁₇H₂₀NOB) C, H, N.

Di-(4-fluorophenyl)borinic acid ethanolamine ester (19ii): Di-(4-fluorophenyl)borinic acid was prepared using method A and was treated with ethanolamine (1M solution in ethanol). A white precipitate formed upon standing and was collected by filtration and washed with ethanol. The purified product had the following properties: mp 247-249° C.; ¹H-NMR (360 MHz, C²H₃₀²H): δ 7.37 (dd, J=8.7, 6.4 Hz, 4H), 6.90 (t, J=8.7 Hz, 4H), 3.95 (d, J=6.4 Hz, 2H), 3.02 (d, J=6.4 Hz, 2H); MS (+ve APCI) m/z 261 ([M+H]⁺, ¹⁰B), 262 ([M+H]⁺, ¹¹B); Anal. (C₁₄H₁₄NOBF₂) C, H, N.

Di-(4-chlorophenyl)borinic acid ethanolamine ester (19iii): Di-(4-chlorophenylborinic acid was prepared using method A and was treated with ethanolamine (1M solution in ethanol). A white precipitate formed upon standing and was collected by filtration and washed with ethanol. The purified product had the following properties: mp 241-242° C.; ¹H-NMR (360 MHz, C²H₃₀²H): δ 7.35 (d, J=8.6, 4H), 7.18 (d, J=8.6, 4H), 3.94 (d, J=6.4 Hz, 2H), 3.02 (d, J=6.4 Hz, 2H); MS (+ve APCI) m/z 293 ([M+H]⁺, ¹⁰B, ³⁵Cl, ³⁵Cl), 294 ([M+H]⁺, ¹¹B, ³⁵Cl, ³⁵Cl), 295 ([M+H]⁺, ¹⁰B, ³⁵Cl, ³⁷Cl), 296 ([M+H]⁺, ¹¹B, ³⁵Cl, ³⁷Cl), 297 ([M+H]⁺, ¹⁰B, ³⁷Cl, ³⁷Cl), 298 ([M+H]⁺, ¹¹B, ³⁷Cl, ³⁷Cl); Anal. (C₁₄H₁₄NOBCl₂) C, H, N.

### EXAMPLE 7

### SEQ ID NO:1: Rhizobium methyltransferase Sequence

a. Isolation

The Rhizobium meliloti ccrM gene (Rhizobium ccrM) was isolated by generating specific probes to Rhizobium ccrM using the Polymerase Chain Reaction (PCR) and using them to screen a R. meliloti lambda library. The primers used to generate the probe had the following sequence: Forward primer (IFADDPPY): 5′-ATY TTY GCB GAY CCB CCB TA (SEQ ID NO:9) Reverse primer 1 (LDPFFG): 5′-CCR MR MV GGR TCS AG (SEQ ID NO:10) Reverse primer 2 (IGIERE): 5′-TCV CGY TCR ATV CCR AT (SEQ ID NO: 11) Forward primer and reverse primer 1 amplify a 570 bp fragment. Forward primer and reverse primer 2 amplify a 635 bp fragment. The R. meliloti lambda library was obtained and subsequent screening was accomplished as described in Sambrook et al.

Three positive clones were isolated from the library. The complete Rhizobium ccrM gene was isolated as a 3.0 kb NotI fragment and has been completely sequenced in both directions (SEQ ID NO:1). The gene encodes a protein having SEQ ID NO:2.

b. Homology between the Caulobacter and Rhizobium ccrM Methyltransferase Genes

The deduced sequences of the Rhizobium and Caulobacter ccrM genes were compared, revealing 61% identity and 74% similarity. The homology is present throughout the two sequences, particularly around regions which had been previously identified as important to the function of other known adenine DNA methyltransferases. However, there are regions of divergence, especially around the N- and C-termini.

The DNA methyltransferase M. Hinfl from Haemophilus influenzae has the same recognition sequence (GANTC) as CcrM and is part of a restriction modification system in this bacteria (Chandrasegaran et a/., Gene 70:387-392, 1988). It should be noted that H. influenzae is not part of the alpha subdivision of gram-negative bacteria and therefore it is likely that this DNA methyltransferase evolved separately from the ccrM family. The deduced sequences derived from the Rhizobium and Caulobacter ccrM genes were compared to the M. Hinfl sequence and it was found, as predicted, that the Caulobacter and Rhizobium genes are much more closely related to each other than to the M. Hinfl DNA methyl-transferase.

% similarity between the Rhizobium (Rh),
Caulobacter (Cc) Bruce/la, Hp Helicobacter pylon and
M. Hinfl (HO CcrM proteins

| | Cc | Rh | Br | Hf | Hp |
|---|---|---|---|---|---|
| Cc | 100 | 74 | 82 | 66 | 57 |
| Rh | | | 90 | 64 | 53 |
| Br | | | | 66. | 54 |
| Hf | | | | | 71 |

c. Rhizobium ccrM is Essential in Rhizobium

Previous work by Stephens et al., Proc. Natl. Acad. Sci. 93:1210-1214, (1996) has demonstrated that the Caulobacter ccrM is essential for viability in Caulobacter. Therefore it is of interest to determine whether other ccrM homologs are also essential.

The coding sequence of the Rhizobium ccrM was disrupted by insertion of the gene encoding kanamycin/neomycin resistance (a selectable marker) into the middle of the gene. This construct was cloned into a suicide plasmid that under selection integrates into the Rhizobium ccrM locus. The result of this integration is that the wild-type copy is separated from the disrupted copy by the vector sequence, which includes the sacB gene. Growth of Rhizobium containing an active sacB gene on sucrose is lethal (Hynes et al., Gene 78:111-120, 1989).

This enables selection for the second recombination event between the disrupted and wild-type copy of ccrM by growth on sucrose. Selection for the event in which only the disrupted copy remained at the ccrM locus occurred only in the presence of a functional copy of ccrM on a replicating plasmid. Thus the Rhizobium ccrM gene is essential for viability in Rhizobium.

| Strain | Plasmid | | |
|---|---|---|---|
| ccrM::nptII | ccrM+ | | |
| LS2590 | none | 0 | 300 |
| LS2591 | none | 0 | 300 |
| LS2590 | pMB440 | 0 | 300 |
| LS2591 | pMB440 | 0 | 300 |
| LS2590 | pRW175 (ccrM+) | 145 | 105 |
| LS2591 | pRW175 (ccrM+) | 192 | 58 |

The Rhizobium ccrM locus can only be disrupted if ccrM is present in trans.

d. Overexpression of the Rhizobium ccrM Gene Results in Defects in Cell Division and Cell Morphology

Caulobacter goes to great lengths to ensure that CçrM is presently only at a specific time of the cell cycle, by regulating the availability of CcrM at two levels: transcription and protein turnover (Stephens et al., J. Bacteriol. 177:1662-1669, 1995; Wright et al., Genes and Development 10:1532-1542, 1996). If this regulation is perturbed by expressing ccrM throughout the cell cycle, the cells exhibit defects in cell division, cell morphology, and the initiation of DNA replication (Zweiger et al., I. Mol. Biol. 235: 472-485, 1994; Wright et at., Genes and Development 10:1532-1542, 1996). Thus it is important to ensure that CcrM is only present in predivisional stage of the Caulobacter cell cycle. We were therefore interested to determine what would happen if the Rhizobium ccrM gene were expressed at high levels in Rhizobium.

The 3.0 kb NotI fragment encompassing the Rhizobium ccrM gene was ligated into a high copy number plasmid and this construct was mated into wild-type Rhizobium. The phenotype of the resulting strain is clearly abnormal compared to wild-type. Wild type Rhizobium is a short rod-shaped cell; however, the cells of the strain in which ccrM was overexpressed are much larger and are highly branched. The branching points appear to occur randomly and vary dramatically between cells. This phenotype is similar to that observed when the cell division gene ftsZ is overexpressed in Rhizobium (B. Margolin, personal communication).

Interestingly, if the Rhizobium ccrM gene is placed in the high copy number plasmid such that it is driven by an additional promoter from the plasmid, no transformants were obtained in Rhizobium. This suggests that the cells can tolerate, to a certain extent, an elevated level of CcrM, but there is a point at which the level of ccrM in the cell becomes lethal.

As CcrM is only present at a specific time in the Caulobacter cell cycle, hemimethylated DNA can be detected in mixed cell cultures. When ccrM is expressed throughout the cell cycle, whether in a Ion null mutant or from expression from a constitutively transcribed promoter, only fully methylated DNA can be detected. It was of interest to determine whether hemimethylated DNA could be detected in Rhizobium, which would suggest that the Rhizobium ccrM is also cell cycle regulated. A naturally occurring restriction site that overlaps a Hinfl site and is sensitive to adenine methylation was identified in Rhizobium. The DNA methylation state at that site was determined and hemimethylated DNA was detected. For a detailed explanation of this experiment see Zweiger et al., J. Mol. Biol. 235: 472-485, (1994). The detection of hemimethylated DNA could be due to either protection from being methylated by a protein binding at that site or the Rhizobium CcrM being present only at a specific time in the cell cycle.

e. Enzyme Purification

BL21 (DE3) hosting pCS255b was streaked from glycerol stock onto an SB (30 g tryptone, 20 g yeast extract, 10 g MOPS, pH 7.5) agar plate containing 200 38 pg/mi amp, and maintained at 37° C. Each 1 L SB/amp (200 pg/mL) culture was inoculated with one single colony at 37° C. until OD600-0.8. Each cell culture was then induced with 0.5 mM IPTG at 37° C. for 1.5-2 hours.

The cells were harvested by centrifugation at 12000 rpm at 4° C. for 20 minutes. Approximately 20 grams of cell paste was obtained from 5 liters of culture. The cells were resuspended in a 25 mM HEPES, pH 7.5, 1 mM EDTA, 5 mM β-mercaptoethanol (βME), 1 mg/mL lysozyme, and 0.1% PMSF 10% glycerol, and lysed by sonication using a SO°lo duty cycle. The process involved sonicating for 30 seconds, stirring the cells for 90 seconds, and repeating the process until the solution was very viscous. This solution was then centrifuged at 12,000 rpm for 20 minutes at 4° C., followed by ultracentrification at 40,000 rpm at 4° C. for 2 hours.

The supernatant was diluted 5-fold with Buffer A (25 mM HEPES, pH 7.5, 5 mM βME, 1 mM EDTA, 10% glycerol) and applied to a 30×2.5 cm DEAE-Sephacel connected to a P11 phosphocellulose column pre-equilibrated with 1 L of buffer A. CcrM does not bind to DEAE-Sephacel while 90% of the proteins from the cell lysate do. The two connected columns were washed with 500 mL buffer A. The P11 column was then disconnected from the DEAE column and eluted with a linear gradient of 1 L buffer A with 25 to 750 mM NaCl. CcrM was eluted at −300 mM NaCl. Fractions were collected and analyzed for protein content by Abs280 as well as by SDS-PAGE.

After elution of the protein from the phosphocellulose column, the enzyme was concentrated using an Amicon apparatus employing a YM-30 molecular weight cut-off membrane. After concentration, the protein was determined to be >95% pure based upon SDS-polyacrylamide gel electrophoresis. The concentration of the protein was first measured using the Bradford colorimetric technique (Bradford, Anal. Biochem. 72, 248-254 (1976)). The second method for determining the concentration of CcrM utilizes measuring the ultraviolet-visible spectroscopy absorbance of the protein at a wavelength of 280 rim. The extinction coefficient of the protein was determined from the predicted amino acid composition (Zweiger et al., J. Mol. Biol. 245, 472-485 (1994)) using the method of Gill and von Hippel Arial. Biochem. 182, 319-326 (1989)). The concentration of CcrM based upon this method is in excellent agreement with the concentration based on the Bradford method.

f. Rhizobium CcrM is Degraded in a Lon Protease-Dependent Process as has been Shown in Caulobacter (Wright et al., Genes and Development 10:1532-1542, 1996).

Lon is a conserved phylogenetically widespread serine protease involved in the degradation of abnormal proteins. We generated a Lon null mutation in Caulobacter crescentus and demonstrated that ccrM transcription is still temporally regulated, but that it is present throughout the cell cycle, resulting in a fully methylated chromosome throughout the cell cycle, causing developmental defects (Wright et al., Genes and Development 10:1532-1542, 1996). Using similar methods as described in Wright et al., we expect that Rhizobium CcrM is degraded in a Lon protease-dependent process as has been shown in Caulobacter.

### EXAMPLE 8

### Brucella abortus Methyltransferase Sequence

The Brucella ccrM gene was isolated using the same strategy and primers as that described for isolating the Rhizobium ccrM gene, but using a Brucella gene library. A specific probe to the Brucella ccrM gene generated by PCR using the above-mentioned primers was used to screen a Brucella lambda library and three clones were isolated.

Restriction mapping of these clones demonstrated that they all contained the full-length ccrM gene. A 2.0 kb HindIII fragment isolated from one of the positive clones which contained the complete Bruce/Ia ccrM gene was sequenced. As with the Rhizobium ccrM gene, the deduced sequence of the Bruce/Ia gene exhibits very high homology to both the Caulobacter and Rhizobium ccrM genes and lower homology to the M. Hinfl DNA methyltransferase.

### EXAMPLE 9

### Agrobacterium tumefaciens Methyltransferase Sequence

The Agrobacterium tumefaciens ccrM gene was isolated using the same strategy as that described for isolating the Rhizobium and Bruce/Ia ccrM gene, but using an Agrobacterium gene library.

### EXAMPLE 10

### Helicobacter pylon Methyltransferase Sequence

Helicobacter pylon is a small, microaerophilic Gram-negative organism which can colonize the human stomach. It is a causative agent of chronic gastritis and peptic ulcer disease, and H. pylon infection has also been epidemiologically correlated with increased risk of gastric carcinoma and lymphoma.

H. pylon belongs to the epsilon subdivision of proteobacteria, and is thus evolutionarily separated from Caulobacter crescentus, Rhizobium meliloti, and Brasilia abortus, all of which belong to the alpha subdivision.

The gene for the H. pylon homolog of CcrM has been cloned and sequenced. Unlike the other ccrM homologs cloned so far, the H. pylon gene has a large open reading frame located immediately downstream. The sequencing of this open reading frame is still in progress. There is high homology between the H. pylon CcrM homolog and the M.Hinfl methyltransferase from Haemophilus influenzae. Because there is extensive precedent for finding close genetic linkage between methyltransferases and their cognate restriction endonucleases in Type II restriction-modification systems such as Hinfl, it is likely that this open reading frame encodes a restriction endonuclease.

Because of the function of methyltransferases in such restriction-modification systems (i.e. protecting native host DNA from digestion by the cognate restriction endonuclease), it is also likely that absence of the functional methyltransferase will prove lethal to H. pylon.

The Helicobacter pylon ccrM gene was isolated using the same strategy as that described for isolating the above ccrM genes, but using a Helicobacter library.

### EXAMPLE 11

### Assay for Methyltransferase

The present invention also comprises efficient assays for determining methyltransferase activity.

a. Materials

[³H]-S-Adenosyl methionine ([³H]-SAM), [γ-³²P]ATP, and [α-³²P]-dATP were from New England Nuclear. Phosphorarnidites for DNA synthesis were obtained from Glenn Research with the exception of the N6-methyl-deoxyadenosine phosphoramidite which was obtained from Pharmacia. Restriction and DNA modifying enzymes used during molecular cloning and DNA manipulation experiments were generally from New England Biolabs, Promega, United States Biochemical, or Boehringer Mannheim. All other materials were obtained from commercial sources and were of the highest available quality.

The CcrM used in the following assays was obtained by the purification protocol described in Example 6.

b. In Vitro Assays

Methyltransferase activity of CcrM was assayed by two distinct methods. In the first method, restriction assays were used to test methylation of restriction sites. The amount of DNA that is resistant to cleavage by restriction enzyme digest due to hemi- or full methylation of either the small DNA substrate or the pUC18 plasmid can be accurately monitored. If the DNA is hemi- or fully methylated by CcrM, the restricted enzyme is unable to cleave the DNA molecule and full length starting material will be obtained. If the DNA is cleaved by the restriction enzyme, smaller DNA fragments will be obtained and indicate a lack of methyl incorporation into the oligonucleotide.

The sequences of the DNA substrates were derived from the upstream sequence from the dnaA promoter. The sequence of the dnaA promoter has been published (Zweiger et al., I. Mol. Biol. 235: 472-485, 1994).

The following is a list of substrates that were used (this list is not meant to be exhaustive):

| | |
|---|---|
| 17/23 mer DNA substrate: | |
| 5′ actcgcgagtcaacaga 3′ | (SEQ ID NO:12) |
| 3′ gagcgctcagttgtctttatCgg 5′ | (SEQ ID NO:13) |
| 23/30-mer | |
| 5- TCC TCT CGC GAG TCA ACA GAA AT | (SEQ ID NO:14) |
| 3′- AGG AGA GCG CTC AGT TGT CTT TAT AGG CGC | (SEQ ID NO:15) |
| N⁶23/30-mer | |
| CH₃ | |
| \| | |
| 5′-TCC TCT CGC GAG TCA ACA GAA AT | (SEQ ID NO:16) |
| 3′- AGG AGA GCG CTC AGT TGT CTT TAT AGG CGC | (SEQ ID NO:15) |
| N⁶23/N⁶30-mer | |
| CH₃ | |
| \| | |
| 5′-TCC TCT CGC GAG TCA ACA GAA AT | (SEQ ID NO:16) |
| 3′-AGG AGA GCG CTC AGT TGT CTT TAT AGG CGC | (SEQ ID NO:17) |
| \| | |
| CH₃ | |
| 45/50..mer | |
| 5′-ATC CTC TCG CGA GTC AAC AGA AAT ATC CGC TCA TCA CCG CAA GTT | (SEQ ID NO:18) |
| 3′- AG GAG AGC GCT CAG TTG TCTTTA TAG GCG AGT AGT GGC GTT CAA TAG GCA A | (SEQ ID NO:19) |
| N⁶45/50-mer | |
| CH₃ | |
| \| | |
| 5′-ATC CTC TCG CGA GTC CCA AGA AAT ATC CGC TCA TCA CCG CAA GTT | (SEQ ID NO:20) |
| 3′- AG GAG AGC GCT CAG TTG TCT TTA TAG GCG AGT AGT GGC GTT CAA AAG GCA A | (SEQ ID NO:21) |
| 60/66-mer | |
| 5′-ATC CTC TCG CGA GTC AAC AGA AAT ATC CGC GAG TCA CCG CAA GTT TTG CGT TTG ACC GGC | (SEQ ID NO:22) |
| 3′- AG GAG AGC GCT CAG TTG TCT TTA TAG GCG CTC AGT GGC GTT CAA AAG GCA AAC TGG CCG | (SEQ ID NO:23) |
| TGG GAG G | |
| N⁶60/66-mer | |
| CH₃ CH₃ | |
| \| \| | |
| 5′-ATC CTC TCG CGA GTC AAC AGA AAT ATC CGC GAG TCA CCG CAA GTT TTC CGT TTG ACC GGC | (SEQ ID NO:24) |
| 3′- AG GAG AGC GCT CAG TTG TCT TTA TAG GCG CTC AGT GGC GTT CAA AAG GCA AAC TGG CCG | (SEQ ID NO:23) |
| TGG GAG G | |

All synthetic oligonucleotides were synthesized using a DNA synthesizer and were purified as previously described by Capson et al., Biochemistry 31, 10984-10994 (1992)). Small duplex DNA substrates (23/30-mer) were prepared by the protocol of Kuchta et al., Biochemistry 26, 8410-8417 (1987)).

Larger DNA substrates (60/66-mer and N660/66-mer) were prepared using a modification of the protocol established by Kaboord and Benkovic, Proc. Natl. Acad. Sci. USA 90, 10881-10885 (1993). Briefly, each single-strand DNA component was constructed by first 5′ labeling one oligonucleotide. After ensuring that the labeling reaction was greater than 95% complete, the labeled oligonucleotide was annealed with the second oligonucleotide and a small linker oligonucleotide to bridge the gapped region. The two oligonucleotides were then ligated in the presence of T4 DNA ligase and MgATP. The linker oligonucleotide was separated from the ligated oligonucleotide by denaturing gel electrophoresis. The complementary large strand was constructed in an identical manner. Following purification of each respective large oligonucleotide, the two strands were annealed and purified by nondenaturing gel electrophoresis described by Capson et al., Biochemistry 31, 10984-10994 (1992). All duplex DNA were quantitated as described by Kuchta et al., Biochemistry 26, 8410-8417 (1987).

Analysis of DNA cleavage depends upon the nature of the DNA substrate. Small duplex DNA substrates can be 5′ end-labeled using bacteriophage T4 polynucleotide kinase and [γ-³²P]ATP as the phosphate source. Both cleaved and uncleaved DNA are resolved by 20% denaturing gel electrophoresis followed by phosphorimaging techniques to analyze for product formation, i.e., cleavage of the larger duplex DNA. Furthermore, accurate quantitation of the reaction products was obtained by manipulation of the Phosphorlmager software.

A typical assay for the methyltransferase activity of .CcrM was performed incubating 50 nM CcrM with 1 pM 5′-labeled DNA while maintaining the concentration of S-adenosyl methionine (SAM) at 20 pM. The reaction was performed in a buffer consisting of 50 mM Tris-HCl, pH 7.5 and 5 mM β-mercaptoethanol (βME) with 150 mM potassium acetate at 30° C. 10 pL aliquots of the methylation reaction were quenched at variable times from 30 seconds to 10 minutes with 10 pL 1 N HCl, extracted with 40 pL of phenol/chloroform, and neutralized with 3 M NaOH in 1 M Tris. The methylated DNA was then subjected to restriction digest by either Hinfl or HindIII. Each reaction contained a final concentration of 100 nM reacted DNA in the presence of 1 unit/pL of Hinfl or HindIII in the appropriate reaction buffer supplied by the manufacturer at 37° C. After 30 minutes, 10 pL of reaction mixture was quenched with 10 pL of gel loading buffer (10% formamide, 0.25% bromophenol blue, and 0.25% xylene cyanol FF). 10 pL of this solution was then run on a 20% sequencing gel to visualize both protection and degradation of the 23/30-mer DNA as a function of time. Product formation was quantitated by measuring the ratio of uncleaved substrate and cleaved product. The ratios of substrate protection are corrected for substrate in the absence of CcrM. Corrected ratios are then multiplied by the concentration of total DNA used in each assay to yield the amount of DNA protected.

Enzymatic assays were also performed using plasmid pUC18 DNA substrate under similar reaction conditions described above. Reaction products using the larger pUC18 substrate were resolved by agarose gel electrophoresis (1% agarose gels). Cleaved and uncleaved DNA are easily visualized under ultraviolet light after staining the gel with 0.5 pg/mL of ethidium bromide. Quantitation of the reaction products for kinetic analysis were performed by densitometry measurements.

A second method involves direct measurement of the incorporation of [³H]-CH₃ from [³H]-SAM into DNA. A typical assay consists of 250 nM CcrM, 5 pM DNA (hemi- or unmethylated) and 6 pM [³H]-SAM in the appropriate reaction buffer. 5 pL aliquots of the reaction are quenched in solution containing 500 pL 10% perchloric acid, 200 pL saturated potassium pyrophosphate, and 20 pL 1 mg/mL single-stranded DNA at times ranging from 15 seconds to 30 minutes. The quenched samples are placed on ice for 30 minutes to precipitate all DNA. The precipitated DNA is then recovered by filtration using glass fiber filters and washed, first with cold 0.1 N HCl (five times with 1.5 mL) and then with cold 95% ethanol (four times with 1.5 mL). The filters are then dried at 90° C. for 10 minutes and counted by standard liquid scintillation techniques. The specific activity of the reaction is determined by measuring the counts per minute present in a fixed quantity of the original reaction in the absence of washing.

Specific activity (SA) was determined by measuring the CPMs present in 5 pL of original reaction. SA CPMs/pmol SAM. The amount of methyl incorporation was determined as follows:

$$\frac{( {CPM5}_{sample} - {CPM}_{zero} )}{Specific Activity} = pmol product$$

The amount of methyl incorporation into the DNA substrate is determined by dividing the counts per minute of the washed reaction samples by the specific activity of the total reaction mixture. This yields product formation in terms of mole quantities. All data are corrected for nonspecific binding of [³H]-SAM to the washed filter.

Alternatively, following the enzymatic incorporation of [³H]-CH3 from [³H]-SAM into DNA, a 5 μl aliquot of the reaction is spotted at variable times onto DES anion-exchange filter paper. The filters are then washed 3 times for 10 minutes with 200 mL 0.3 M ammonium formate, pH 8 to remove unreacted [³H]-SAM. The filters are then briefly washed twice with 95% ethanol and then washed once with anhydrous ether. The filters are then air dried and counted by standard liquid scintillation techniques. The specific activity of the reaction is determined by measuring the radioactivity present in 5 pL of the reaction spotted on glass filter fibers without washing. The amount of methyl incorporation into the DNA substrate is determined by diving the counts per minute of the washed samples by the specific activity of the total reaction mixture, yielding product formation in terms of pmol quantities. all data are corrected for nonspecific binding of [³H]-SAM to the washed filter.

During the course of performing the above assays, it was observed that: the N6-23/30-mer N645/50-mer, and the N6-60/66-mer are preferred substrates by ratios of 10:1 and 2:1; the tested methyltransferases are processive under the assay conditions used; optimal activity was at 30° C. rather than 37° C.; and the tested enzymes are DNA-dependent (i.e., they become inactivated in the solutions used after about 20 minutes in the absence of DNA substrate). The loss of activity in the absence of a substrate does not appear to involve proteolytic degradation.

C. In Vivo Assay

A single colony of BL21(DE3) or DH5a hosting pCS255b was used to inoculate a 5 mL SB/amp (200 pg/mL) overnight culture at 37° C. The BL2I(DE3) culture was divided into two aliquots at OD600-1. One aliquot was induced with 1 mM IPTG at 37° C. overnight while the other was allowed to grow without induction. Cell cultures were centrifuged, from which cell pellets were subjected to mini plasmid prep. The recovered plasmids from DH5a and MIME) (with and without IPTG induction) were digested with Hinfl and the restriction digests were analyzed by 1% agarose gels. In all cases, controls containing the undigested plasmid were included. Plasmid recovered from DI-15a was susceptible to Hinfl digestion while plasmids from BL21 (DE3) with and without induction were resistant to Hinfl digestion. It appears that even uninduced BL21(DE3) expresses ccrM. To ascertain that BL21(DE3) did not have intrinsic methyltransferase specific for the GANTC sites, pUC18 was introduced into BL21(DE3). pUC18 recovered from BL21 (DE3) was susceptible to Hinfl digestion, thereby excluding the possibility of BL21(DE3) host cells containing intrinsic M. Hinfl methyltransferase activity.

Although the foregoing invention has been described in some detail by way of illustration and example for purposes of clarity of understanding, it will be obvious that certain changes and modifications may be practiced within the scope of the appended claims. All publications, patents and patent applications mentioned in this specification are hereby incorporated by reference for all purposes, to the same extent as if each individual publication, patent or patent application had been specifically and individually indicated to be incorporated by reference.

### EXAMPLE 12

### Biological Activity Assay

The antibacterial activity of the compounds prepared as descried above were tested using Caulobacter crescentus and Bacillus subtilis as follows.

Caulobacter crescentus Cell Growth Assay

Caulobacter crescentus (CB15N) was grown in PYE media (Sambrook et al., 1989, Molecular Cloning: A Laboratory Manual, 2d ed., Cold Spring Harbor Press: N.Y.) overnight at 30° C. to saturation. Taking advantage of the inherent ampicillin resistance of Caulobacter crescentus to minimize the risk of contamination, this saturated culture was diluted in PYE media containing 200 μg/mL ampicillin to a final OD₆₀₀ of 0.05. Aliquots (146 μL) of this diluted cell culture were placed in wells of a microtiter plate. An inhibitor (4 μL of a stock solution of an appropriate concentration dissolved in either dimethylformamide or dimethylsulfoxide) was added to each of these wells to give a final volume of 150 μL. This plate was incubated at 30° C. with gentle shaking at 550 rpm in an Eppendorf Thermomixer R. Control experiments were performed in parallel. These consisted of wells containing: i) 150 μL PYE/ampicillin media (no cell culture) as a blank; and ii) 146 μL diluted cell culture and 4 μL (DMF or DMSO) for maximum cell growth in the presence of solvent and absence of inhibitor. Cell growth was monitored at 630 nm using a microtitre plate reader at time points of: 0 hours, 4 hours, 8 hours, and 22 hours. The final cell growth was recorded as a percent of the maximum cell growth.

Bacillus subtilis Cell Growth Assay

A similar cell growth assay to that described for Caulobacter crescentus was performed with Bacillus subtilis (ATCC #33234). The following changes were incorporated to accommodate the different growth conditions: i) cells were grown in Luria-Bertani (LB) media without antibiotics (Sambrook et al., ibid.); ii) growth temperature was 37° C.; and iii) cell growth was monitored at time points of 0 hours, 2.5 hours, 5 hours, and 7.5 hours. After 7.5 hours any culture with inhibited cell growth was diluted 500-fold into fresh LB media. Recovery from inhibitor selection was assessed after 12 hours at 37° C.

CcrM Inhibition Assay

Methyltransferase activity was measured by monitoring the incorporation of [³H]CH₃ from [³H]-S-adenosylmethionine (SAM) into DNA. A stock solution containing 250 nM CcrM, 5 μM N645/50 mer (the sequence of which is identified below), 150 mM potassium acetate, 5 mM 2-mercaptoethanol in pH 7.5 HEPES buffer was prepared. Aliquots were placed in Eppendorf tubes and inhibitors were added from concentrated stock solutions (16.7 mM in DMF or DMSO) to reach the appropriate final concentrations (500 μM or 100 μM) in a total of 15 μL. Reactions were initiated by the addition of [³H]SAM at a final concentration of 50 μM. After 40 mins at 30° C., 5 μL aliquots were removed from the reaction and spotted onto DE81 anion exchange filter circles. The filters were allowed to dry and then washed with 3×200 mL of 0.3 M ammonium formate to remove unreacted [³H]SAM, followed by 2×200 mL 95% ethanol wash and finally a 200 mL ether wash. The filters were allowed to air dry and counted by standard liquid scintillation techniques. Control reactions in the absence of inhibitors were used to determine the extent of inhibition. High throughput screening was carried out similarly in Tris-HCl buffer (50 μM, pH 7.5) with plasmid DNA as substrate (Litmus 29 (New England Biolabs): 250 μM DNA, 3 μM sites), 100 μM inhibitor candidate and enzyme, potassium acetate and 2-mercaptoethanol concentrations as described above. Assays were initiated with [¹⁴CH₃]SAM (50 μM, 34 Ci/mol) in a volume of 10 μL in a PCR plate, and incubated for 30 minutes at 30° C. Four microliter aliquots were then spotted on DE81 paper with a multichannel pipette and washed and dried as described above. Data was collected with a Molecular Dynamics model 425S phosphorimager, and analyzed with the spotfinder utility in ImageQuant 3.3.

```
DNA substrate [N645/50 mer]
                                  CH₃
                                  |
5′-ATC CTC TCG CGA ATC AAC AGA AAT ATC CGC TCA TCA CCG CAA GTT
3′- AG GAG AGC GCT TAG TTG TCT TTA TAG GCG AGT AGT GGC GTT CAA AAG GCA A
```

(where the methylated strand shown above is SEQ ID No. 1 and the complementary strand is SEQ ID No. 2). Synthesis of DNA was achieved on an Expedite BioSystems DNA synthesizer and purified as previously described (Capson et al., 1992, Biochemistry 31: 10984-10994).

TABLE I

| | |
|---|---|
| % reaction | IC₅₀ (μM) |

| | Brucella | Caulobacter | Bacillus |
|---|---|---|---|
| | abortus | crescentus | Subtilis |
| Compound | CcrM Activity | Cell Growth | Cell Growth |
| (11i) | | >100 | nt |
| (11ii) | 61 | 23 | nt |
| (11iii) | 61 | 16 | nt |
| (11iv) | | 10 | <10 |
| (11v) | | 26 | <10 |
| (11vi) | 42 | 32 | 28 |
| (11vii) | | 24 | 20 |
| (11viii) | 74 | >100 | 28 |
| (12ii) | 17 | 56 | >100 |
| (12iii) | 40 | 5 | 24 |
| (12vii) | 56 | 24 | >100 |
| (13ii) | 55 | 7 | 27 |
| (13iii) | 81 | 6 | <10 |
| (13vi) | | 7 | >100 |
| (13vii) | | 7 | 36 |
| (14vi) | 21 | 7 | >100 |
| (15i) | 38 | >100 | >100 |
| (16i) | 93 | >100 | >100 |
| (17i) | 114 | >100 | >100 |
| (18i) | 81 | >100 | >100 |
| (19ii) | 8 | 85 | nt |
| (19iii) | | 17 | nt |

These results indicate that the compounds of the invention have useful IC50 values for inhibiting CcrM and DAM methylases in bacteria.

These compounds have advantageous physical properties, and are isolated as pure, stable solids that are amenable to large-scale production. Additional specific embodiments of adenine DNA methyltransferase inhibitors of the invention includes related compounds having these additional features:
1) Analogues with various substituents on the phenyl rings in any, or combination of, the ortho-, meta- and para-positions, including fused rings and substituted fused rings;
2) Analogues having aromatic heterocycles of various ring sizes, substituted heterocycles, fused heterocycles and substituted fused heterocycles in place of one or both phenyl groups;
3) Analogues having two non-identical aromatic rings bound to the boron atom, using combinations of the aromatic systems described in 1) and 2) above;
4) Analogues prepared using quinolines (9) containing various substituents in any possible position or structural analogues including fused heteroaromatic rings containing one or more heteroatom in any possible position or fused heteroaromatic rings containing one or more heteroatom in any possible position and containing various substituents in any possible position; and
5) Analogues having substitutions on either, or both of, the C-1 and C-2 positions of the ethylene group of the 2-aminoethanol of (10).

It should be understood that the foregoing disclosure emphasizes certain specific embodiments of the invention and that all modifications or alternatives equivalent thereto are within the spirit and scope of the invention as set forth in the appended claims.

1. A method for treating or protecting an animal against a microbe-induced disease comprising the step of inhibiting DNA methyltransferase activity in said microbe.

2. The method of claim 1 wherein said DNA methyltransferase is a DNA adenine methyl transferase.

3. The method of claim 1 wherein said inhibiting DNA methyltransferase activity results from inhibiting DNA methyltransferase enzyme activity.

4. The method of claim 1 wherein said inhibiting DNA methyltransferase activity results from inhibiting expression of DNA methyltransferase.

5. The method of claim 1 wherein said animal is a human patient.

6. The method of claim 1 wherein said microbe is a bacterium.

7. The method of claim 6 wherein said bacterium is a gram positive bacterium.

8. The method of claim 7 wherein said gram positive bacterium is a bacterium of Staphylococcus species, Streptococcus species, Bacillus species, Corynebacterium species, Clostridium species, Actinomyces species, Enterococcus species, or Streptomyces species.

9. The method of claim 6 wherein said bacterium is a gram negative bacterium.

10. The method of claim 9 wherein said gram negative bacterium is a bacterium of Acinetobacter species, Neisseria species, Pseudomonas species, Brucella species, Agrobacterium species, Bordetella species, Escherichia species, Shigella species, Yersinia species, Salmonella species, Klebsiella species, Enterobacter species, Hemophilus species, Pasteurella species, Streptobacillus species, spirochetal species, Campylobacter species, Vibrio species, or Helicobacter species.

11. The method of claim 6 wherein said bacterium is a bacterium of species Staphylococcus aureus; Staphylococcus saprophyticus; Streptococcus pyogenes; Streptococcus agalactiae; Streptococcus pneumoniae; Enterococcus faecalis; Enterococcus faecium; Bacillus anthracis; Acinetobacter baumanii; Corynebacterium diphtheria; Clostridium perfringens; Clostridium botulinum; Clostridium tetani; Neisseria gonorrhoeae; Neisseria meningitidis; Pseudomonas aeruginosa; Legionella pneumophila; Escherichia coli; Yersinia pestis; Haemophilus influenzae; Helicobacter pylori; Campylobacter fetus; Vibrio cholerae; Vibrio parahemolyticus; Trepomena pallidum; Actinomyces israelii; Rickettsia prowazekii; Rickettsia rickettsii; Chlamydia trachomatis; Chlamydia psittaci; Brucella abortus; Agrobacterium tumefaciens; or Francisella tularensis.

12. A method of treating or protecting against a microbe-induced disease in a mammal afflicted with said disease, or at risk of becoming afflicted with said disease, comprising administering to said mammal a therapeutically effective dose of a methyl transferase inhibitor.

13. The method of claim 12 wherein said DNA methyltransferase is a DNA adenine methyl transferase.

14. The method of claim 12 wherein said inhibiting DNA methyltransferase activity results from inhibiting DNA methyltransferase enzyme activity.

15. The method of claim 12 wherein said inhibiting DNA methyltransferase activity results from inhibiting expression of DNA methyltransferase.

16. The method of claim 12 wherein said animal is a human patient.

17. The method of claim 12 wherein said microbe is a bacterium.

18. The method of claim 17 wherein said bacterium is a gram positive bacterium.

19. The method of claim 18 wherein said gram positive bacterium is a bacterium of Staphylococcus species, Streptococcus species, Bacillus species, Corynebacterium species, Clostridium species, Actinomyces species, Enterococcus species, or Streptomyces species.

20. The method of claim 17 wherein said bacterium is a gram negative bacterium.

21. The method of claim 20 wherein said gram negative bacterium is a bacterium of Acinetobacter species, Neisseria species, Pseudomonas species, Brucella species, Agrobacterium species, Bordetella species, Escherichia species, Shigella species, Yersinia species, Salmonella species, Klebsiella species, Enterobacter species, Hemophilus species, Pasteurella species, Streptobacillus species, spirochetal species, Campylobacter species, Vibrio species, or Helicobacter species.

22. The method of claim 17 wherein said bacterium is a bacterium of species Staphylococcus aureus; Staphylococcus saprophyticus; Streptococcus pyogenes; Streptococcus agalactiae; Streptococcus pneumoniae; Enterococcus faecalis; Enterococcus faecium; Bacillus anthracis; Acinetobacter baumanii; Corynebacterium diphtheria; Clostridium perfringens; Clostridium botulinum; Clostridium tetani; Neisseria gonorrhoeae; Neisseria meningitidis; Pseudomonas aeruginosa; Legionella pneumophila; Escherichia coli; Yersinia pestis; Haemophilus influenzae; Helicobacter pylon; Campylobacter fetus; Vibrio cholerae; Vibrio parahemolyticus; Trepomena pallidum; Actinomyces israelii; Rickettsia prowazekii; Rickettsia rickettsii; Chlamydia trachomatis; Chlamydia psittaci; Brucella abortus; Agrobacterium tumefaciens; or Francisella tularensis.

23. A method of reducing bacterial virulence, comprising contacting bacteria with an agent that alters the bacteria's native level of DNA methyltransferase activity thereby inhibiting virulence of the bacteria.

24. The method of claim 23 wherein contacting bacteria with an agent that alters the bacteria's native level of DNA methyltransferase activity results in altering the bacteria's native level of methylation of adenine in a polynucleotide of said bacteria.

25. The method of claim 23 wherein contacting bacteria with an agent that alters the bacteria's native level of DNA methyltransferase activity results in altering the bacteria's native level of methylation of adenine in a GATC tetranucleotide of the bacteria.

26. The method of claim 23 wherein contacting bacteria with an agent that alters the bacteria's native level of DNA methyltransferase activity results in altering the bacteria's native level of methylation of adenine in a GANTC pentanucleotide of the bacteria.

27. The method of claim 23 wherein the bacteria are pathogenic bacteria that cause disease in a mammal.

28. The method of claim 23 wherein the agent reduces the DNA methyltransferase activity.

29. The method of claim 28 wherein said agent reduces said activity by binding to a DNA methyltransferase enzyme.

30. The method of claim 23 wherein said DNA methyltransferase is a DNA adenine methyl transferase.

31. The method of claim 23 wherein said inhibiting DNA methyltransferase activity results from inhibiting DNA methyltransferase enzyme activity.

32. The method of claim 23 wherein said inhibiting DNA methyltransferase activity results from inhibiting expression of DNA methyltransferase.

33. The method of claim 23 wherein said animal is a human patient.

34. The method of claim 23 wherein said microbe is a bacterium.

35. The method of claim 23 wherein said bacterium is a gram positive bacterium.

36. The method of claim 23 wherein said gram positive bacterium is a bacterium of Staphylococcus species, Streptococcus species, Bacillus species, Corynebacterium species, Clostridium species, Actinomyces species, Enterococcus species, or Streptomyces species.

37. The method of claim 23 wherein said bacterium is a gram negative bacterium.

38. The method of claim 23 wherein said gram negative bacterium is a bacterium of Acinetobacter species, Neisseria species, Pseudomonas species, Brucella species, Agrobacterium species, Bordetella species, Escherichia species, Shigella species, Yersinia species, Salmonella species, Klebsiella species, Enterobacter species, Hemophilus species, Pasteurella species, Streptobacillus species, spirochetal species, Campylobacter species, Vibrio species, or Helicobacter species.

39. The method of claim 23 wherein said bacterium is a bacterium of species Staphylococcus aureus; Staphylococcus saprophyticus; Streptococcus pyogenes; Streptococcus agalactiae; Streptococcus pneumoniae; Enterococcus faecalis; Enterococcus faecium; Bacillus anthracis; Acinetobacter baumanii; Corynebacterium diphtheria; Clostridium perfringens; Clostridium botulinum; Clostridium tetani; Neisseria gonorrhoeae; Neisseria meningitidis; Pseudomonas aeruginosa; Legionella pneumophila; Escherichia coli; Yersinia pestis; Haemophilus influenzae; Helicobacter pylon; Campylobacter fetus; Vibrio cholerae; Vibrio parahemolyticus; Trepomena pallidum; Actinomyces israelii; Rickettsia prowazekii; Rickettsia rickettsii; Chlamydia trachomatis; Chlamydia psittaci; Brucella abortus; Agrobacterium tumefaciens; or Francisella tularensis.

40. A method of reducing bacterial virulence, comprising:
contacting bacteria with an agent that alters the bacteria's native level of DNA methyltransferase activity thereby altering the bacteria's native level of methylation of adenine in a GATC tetranucleotide of the bacteria, and thereby inhibiting virulence of the bacteria.

41. A method of treating a microbe-induced condition in a mammal afflicted with said condition, comprising administering to said mammal a therapeutically effective dose of a composition comprising a methyl transferase inhibitor and a pharmacological excipient.

42. The method of claim 41 wherein said condition is caused by Agrobacterium spp., Rhizobium spp. or Helicobacter spp.

43. The method of claim 41 wherein said condition is caused by a member of the alpha subdivision of gram-negative bacteria.

44. The method of claim 41 wherein said mammal is a human.
